(19) United States
(12) Patent Application Publication
(10) Pub. No.: US 2025/0034630 A1
(43) Pub. Date: Jan. 30, 2025

(54) METHODS FOR ANALYZING CHROMOSOMES AND CHROMOSOMAL ABNORMALITIES USING dGH WITH FLUORESCENCE SORTING AND/OR ARRAYS

(71) Applicant: KromaTiD, Inc., Longmont, CO (US)

(72) Inventors: Erin Marie CROSS, Fort Collins, CO (US); Stephen William HUGHES, Longmont, CO (US); Lauren KINNER, Longmont, CO (US); Christopher John TOMPKINS, Longmont, CO (US)

(73) Assignee: KromaTiD, Inc., Longmont, CO (US)

(21) Appl. No.: 18/714,564

(22) PCT Filed: Dec. 1, 2022
(86) PCT No.: PCT/US2022/080780

§ 371 Date: May 29, 2024

Related U.S. Application Data
(60) Provisional application No. 63/366,601, filed on Jun. 17, 2022.
(60) Provisional application No. 63/366,142, filed on Jun. 9, 2022.
(60) Provisional application No. 63/264,764, filed on Dec. 1, 2021.

(51) Int. Cl. C12Q 1/6841; C12Q 1/6827

(52) U.S. Cl. CPC C12Q 1/6841; C12Q 1/6827

(57) ABSTRACT

The present disclosure provides methods and compositions for analyzing enriched populations of cells or target chromosomes, for example cells in metaphase and methods and compositions for two-dimensional spatial arrangement of cells and chromosomes. Furthermore, methods are disclosed for the detection of structural variations and/or repair events in chromosomes by labeling of single-stranded chromatids with probes, which in illustrative embodiments are of different colors. The hybridization pattern of the labeled probes produces a spectral pattern that provides high-resolution detection of structural variations and/or repair events, which for example can facilitate distinction of benign structural variations from deleterious structural variations. Further, the spectral pattern provides information regarding complex structural variations where more than one rearrangement of chromosomal segments may have occurred. Spectral information can be used to generate data tables upon which nodal analysis can be applied to identify structural features of interest.

### CROSS-REFERENCE TO RELATED APPLICATIONS

This patent application claims priority from U.S. Provisional Application No. 63/264,764, filed Dec. 1, 2021, U.S. Provisional Application No. 63/366,142, filed Jun. 9, 2022, and U.S. Provisional Application No. 63/366,601, filed Jun. 17, 2022.

### TECHNICAL FIELD

The present disclosure relates generally to detection of structural features in chromosomes using fluorescent probes and fluorescence analysis, and, more particularly, to methods for preparation and analysis of chromosomes and cells under study.

### BACKGROUND

Directional genomic hybridization (dGH) is a single cell method for mapping the structure of a genome on single stranded metaphase chromosomes. dGH techniques can facilitate detection of a wider range genomic structural variants than was previously possible.

One manner in which chromosomes are prepared for dGH is the CO-FISH technique. CO-FISH, developed in the 1990s, permits fluorescent probes to be specifically targeted to sites on either chromatid, but not both. In “Strand-Specific Fluorescence in situ Hybridization: The CO-FISH Family” by S. M. Bailey et al., Cytogenet. Genome Res. 107: 11-14 (2004), chromosome organization is studied using strand-specific FISH (fluorescent or fluorescence in situ hybridization) (CO-FISH; Chromosome Orientation-FISH) which involves removal of newly replicated strands from the DNA of metaphase (mitotic) chromosomes, resulting in one single-stranded target DNA being present in each mitotic chromatid in which the base sequence in each chromatid is the complement of that of the other. This is achievable because each newly replicated double helix present in the new chromatids contains one parental DNA strand plus a newly synthesized strand, and it is this newly synthesized strand that is removed because it has been rendered photosensitive during replication.

Structural variants (SVs) are broadly defined as changes to the arrangement or order of segments of a genome as compared to a “normal” genome. Simple variants include single occurrences of unbalanced translocations, balanced translocations, homologous translocations, inversions, duplications, insertions, and deletions. Complex variants include multiple simple variants in a single cell, simple variants combined with the loss or gain of genomic material, loss or gain of entire chromosomes and more general DNA damage described as chromothripsis. Heterogeneity of variants, defined as different structural variants appearing in genomes individual cells of the same organism, cell culture or batch of cells can involve simple or complex structural variants. A mosaic of structural variants occurs when dividing cells spontaneously develop a structural variant and both the variant free parent and the daughter containing the variant continue to propagate.

Structural variants are distinguished from base level changes such as single nucleotide polymorphisms (SNiPs) or short insertions and deletions (INDELs). Structural variants occur when the ends of multiple double strand breaks are incorrectly rejoined or mis-repaired. Depending on the subsequent reproductive viability of the cell bearing the rearrangement the consequence of a resulting structural variant can be limited to a single cell, affect a sub-set of the tissues in an organism, or if it occurs in a germ cell, may even be inherited and affect the lineage of the organism.

The potential for DNA mis-repair that leads to chromosome structural variants exists whenever DNA double-strand breaks (DSBs) occur. DSBs can arise endogenously during normal cellular metabolic processes, such as replication and transcription. It has been estimated that DSBs occur naturally at a rate of ˜50 per cell, per cell cycle in actively metabolizing cells, and repair occurs both during replication and through replication-independent pathways. Double strand breaks are of particular concern when induced by exogenous factors above spontaneous rates either through radiation exposure, medical interventions such as chemotherapy with certain agents, or during gene editing processes. Most DSBs are repaired by Non-Homologous End Joining (NHEJ) which operates throughout the cell cycle. In this process the broken ends are detected, processed, and ligated back together. This is an “error-prone” process because the previously existing base-pair sequence is not always restored with high fidelity. Nevertheless, this rejoining process (restitution) restores the linear continuity of the chromosome and does not lead to structural abnormalities. However, if two or more DSBs occur in close enough spatial and temporal proximity the broken end of one break-pair may mis-rejoin with an end of another break-pair, along with the same with the other two loose ends, resulting in a structural abnormality from the exchange. Examples include balanced and unbalanced translocations, inversions, or deletions. There is also a DSB repair process involving Homologous Recombination (HR) sometimes referred to as Homology Directed Repair (HDR). Homology directed repair (HDR) occurs post-replication when an identical homologous sequence becomes available and is in close proximity. The HDR pathway does not operate in G1 or G0 cells where the level of rad51 protein, necessary for HDR is very low or absent. However, as part of the process of gene editing (such as in the CRISPR system) the sequence to be edited is targeted and one or more DSBs are introduced to insert the desired sequence using HDR. If at any time multiple DSBs exist con-currently within a cell, there is a potential for two or more DSBs to be mis-aligned during repair, forming a rearrangement or structural variant. Structural variants are associated with a multitude of human diseases in large part because they can lead to copy number variation, fusion genes, knock downs, knockouts or otherwise significantly impact the function or regulation of genes. The contribution of structural variants to genetic variation is estimated to be 10-30 times higher than SNiPs or INDELs. Thus, high resolution methods for detecting structural variants are needed for detecting chromosomal aberrations and distinguishing benign genetic variations from deleterious genetic abnormalities.

These structural variants, however, they are formed, can be harmless and show no genotoxicity, can negatively affect cellular function, can cause genomic instability, kill the cell, or can form genotoxic products. Non-harmless structural variants negatively affect cells and contribute to disease through the formation of oncogenes; gene inactivation or knock out; regulatory element disruption; loss of heterozygosity; duplication of genes or promotors; and other mechanisms that disrupt necessary metabolic pathways or activate inert metabolic pathways. If the structural variation is congenital, even if it does not result in any obvious pathology, mistakes in meiotic crossover caused by misalignment can produce genetic abnormalities in the offspring of the affected individual.

In a typical mendelian fashion, recessive structural variants inherited from both parents can cause disease in children not active in either parent. X-linked structural variations selectively impact male offspring, because the Y chromosome of the XY pair does not have a compensating normal gene.

The detection and identification of both non-recurrent SVs in individual cells resulting from DSB mis-repair, as well as the SVs present in an individual genome and their representation in individual cells (heterogeneity/mosaicism) is clinically relevant and important across a wide spectrum of human disease and conditions. Because of the potential for both cell death and risk to patients DNA, mis-repairs and the resulting structural variants must be measured.

To detect structural variants, two types of approaches are generally employed, array-based detection/comparative genome hybridization (array cGH), and sequence based computational analysis. Next-generation and Sanger sequencing methods have attempted to provide this data through short and long read whole genome sequencing and analysis, but are insufficient and as such serve best as a confirmation of a known structural variation developed by direct measurement. Each can measure some products of mis-repair through SV detection algorithms and can be more effective when used in concert to cross-validate findings. As these techniques measure the sequence of DNA bases and not the relationship or structure of the genes, promotors or large segments of DNA in single cells, they can be used only to hypothesize genomic structure through bioinformatic reconstruction. For targeted measurement of known structural variants, sequence-based methods can sometimes be sufficient, but de novo measurement of structural variation with sequence-based methods has been shown to yield numerous false positive and false negative results, making the technique generally impractical.

The number of samples that can be processed with biological assays in the study of chromosomal features for example in chromosomal imaging analysis, is commonly limited by lab capacity in terms of equipment and personnel. Methods are needed in the art to manage throughput capacity to provide for high-throughput analysis of samples, in the context of existing equipment and personnel capacity. Furthermore, methods are needed for production of high-resolution results from samples studied.

### SUMMARY

To overcome the above-mentioned and additional problems in the art, the present disclosure provides high throughput methods for the detection of chromosomal structural features such as structural variants and repair events.

Provided herein are methods for analyzing cells in a cell population. The methods can include, for example:
sorting cells in the cell population using a cell sorting method, to increase the proportion of cells in metaphase, thereby providing a metaphase-enriched cell population and performing dGH on the metaphase-enriched cell population.

In further aspects, the present disclosure provides methods for two-dimensional spatial arrangement of cells, comprising cells in metaphase. Such arrays form separate aspects herein.

Methods and arrays herein in illustrative embodiments are used in chromosomal imaging. Thus, such methods typically utilize fluorescence microscopy.

Further details regarding aspects and embodiments of the present disclosure are provided throughout this patent application. Sections and section headers are for ease of reading and are not intended to limit combinations of disclosure, such as methods, compositions, and kits or functional elements therein across sections. Further details regarding aspects and embodiments of the present disclosure are provided throughout this patent application. Sections and section headers are for ease of reading and are not intended to limit combinations of disclosure, such as methods, compositions, or other functional elements therein across sections.

### BRIEF DESCRIPTION OF THE DRAWINGS

The patent or application file contains at least one drawing shown in gray scale, wherein the shades of gray represent different colors. Copies of this patent or patent application publication with color drawing(s) will be provided by the Office upon request and payment of the necessary fee.

FIG. 1A-FIG. 1D illustrate an example of intra-chromosomal rearrangements comparing banded dGH paint vs. monochrome dGH paint. FIG. 1A(i): Normal chromosome 2, prepared for dGH, hybridized with Ch 2 dGH paint with multi-color bands. FIG. 1A(ii): Ch 2 with a deletion, bands missing are identified. FIG. 1C(i): Ch 2 with an amplification, region with extra bands identified. FIG. 1C(ii): Ch 2 with a sister chromatid recombination event (only visible for 1 replication cycle—perfect repair event) identified as a SCR due to the bands being in the correct order (not inverted). FIG. 1C(iii): Ch 2 with an inversion event, identified via the inverted order of the bands. FIG. 1B(i): Normal chromosome 2, prepared for dGH, hybridized with monochrome Ch 2 dGH paint. FIG. 1B(ii): Ch 2 with a deletion, region unknown. FIG. 1D(i): Ch 2 with an amplification, region amplified unknown. FIG. 1D(ii): Ch 2 with either an SCR or Inversion event, specific variant unknown. (SCR is potentially missed, flagged as inversion because orientation of the segment seen on the opposite sister chromatid is unknown.) FIG. 1D(iii): Ch 2 with either an SCR or Inversion event, specific variant unknown. (Inversion is potentially missed, flagged as SCR because orientation of the segment seen on the opposite sister chromatid is unknown). The color channel map, shown in gray scale, for individual dGH bands (1-19) is provided in FIG. 10A.

FIG. 2A-FIG. 2D illustrate an example of inter-chromosomal rearrangements (translocations between two different chromosomes), banded dGH paint vs monochrome dGH paint. FIG. 2A(i): Normal Chromosome 2, prepared for dGH, hybridized with Ch 2 dGH paint with multi-color bands. FIG. 2A(ii): Normal Chromosome 4, un-painted for illustration purposes. FIG. 2C(i): Derivative Chromosome A (product of reciprocal translocation), with material from Ch 2 (bands 1-11) fused with material from Ch 4 (unpainted). FIG. 2C(ii): Derivative Chromosome B (other product of reciprocal translocation), with material from Ch 2 (bands 12-19) fused with material from Ch 4 (unpainted). FIG. 2B(i): Normal Chromosome 2, prepared for dGH, hybridized with monochrome Ch 2 dGH paint. FIG. 2B(ii): Normal Chromosome 4, un-painted for illustration purposes. FIG. 2D(i): Derivative Chromosome A (product of reciprocal translocation), with material from Ch 2 fused with material from Ch 4 (unpainted)—coordinates of fusion unknown. FIG. 2D(ii): Derivative Chromosome B (other product of reciprocal translocation), with material from Ch 2 fused with material from Ch 4 (unpainted)—coordinates of fusion unknown. The color channel map, shown in gray scale, for individual dGH bands (1-19) is provided in FIG. 10A.

FIG. 3A-FIG. 3D illustrate an example of inter-chromosomal allelic rearrangements (translocations between two homologs of the same chromosome). Banded dGH paint vs monochrome dGH paint. FIG. 3A(i): Normal Chromosome 2 homolog 1, prepared for dGH, hybridized with Ch 2 dGH paint with multi-color bands. FIG. 3A(ii): Normal Chromosome 2 homolog 2, prepared for dGH, hybridized with Ch 2 dGH paint with multi-color bands. FIG. 3C(i): Derivative Chromosome A (product of reciprocal translocation between homologs), with material from Ch 2 homolog 1 exchanged with material from Ch2 homolog 2 at the same breakpoint (between bands 11 and 12). Statistical chances of two SCEs at the exact same location on each homolog is very unlikely, vs an allelic translocation event being quite likely—especially in a cell being edited at a single location (two DSBs—one per homolog). FIG. 3C(ii): Derivative Chromosome B (product of reciprocal translocation between homologs), with material from Ch 2 homolog 1 exchanged with material from Ch2 homolog 2 at the same breakpoint (between bands 11 and 12). Statistical chances of two SCEs at the exact same location on each homolog is very unlikely, vs an allelic translocation event being quite likely—especially in a cell being edited at a single location (two DSBs—one per homolog). FIG. 3B(i): Normal Chromosome 2 homolog 1, prepared for dGH, hybridized with monochrome Ch 2 dGH paint. FIG. 3B(ii): Normal Chromosome 2 homolog 2, prepared for dGH, hybridized with monochrome Ch 2 dGH paint. FIG. 3D(i): Derivative Chromosome A (product of reciprocal translocation between homologs), with material from Ch 2 homolog 1 exchanged with material from Ch2 homolog 2 at unknown breakpoints. Statistical chances of two SCEs at the exact same location on each homolog is very unlikely, versus an allelic translocation event being quite likely—especially in a cell being edited at a single location (two DSBs—one per homolog), but cannot be confirmed with monochrome paint due to lack of genomic coordinate specificity. FIG. 3D(ii): Derivative Chromosome B (product of reciprocal translocation between homologs), with material from Ch 2 homolog 1 exchanged with material from Ch2 homolog 2 at unknown breakpoints. Statistical chances of two SCEs at the exact same location on each homolog is very unlikely, versus an allelic translocation event being quite likely—especially in a cell being edited at a single location (two DSBs—one per homolog), but cannot be confirmed with monochrome paint due to lack of genomic coordinate specificity. The color channel map, shown in gray scale, for individual dGH bands (1-19) is provided in FIG. 10A.

FIG. 4A-FIG. 4E illustrate an example of using dGH multi-color banding to detect complex chromosomal rearrangements that are difficult to detect using single color dGH. FIG. 4A is a pair of images of chromosome 2 with representations of banded dGH fluorescence patterns overlayed on top of stained images of chromosome 2. In FIG. 4A, both Chromosome 2 homologs from a blood-derived lymphocyte cell recently exposed to ionizing radiation for prostate cancer treatment are shown. Complex structural variations are present on the right homolog, which can be visualized after hybridization with dGH probe that form a banded dGH paint. The arrow in the right image of FIG. 4A points to a band that corresponds to a small paracentric inversion. The diagrams provided in FIG. 4B-FIG. 4E illustrate how a normal chromosome and this complex rearrangement would appear using the multi-color banded dGH paint compared to a monochrome dGH paint. FIG. 4B provides a diagram of a normal chromosome 2 showing target DNA sequences illustrated as gray scale bands (1-19) representing a chromosome 2 dGH paint with multi-color bands. FIG. 4C provides a corresponding multi-color paint diagram of a chromosome 2 with complex structural rearrangements as labeled. FIG. 4D shows a normal Chromosome 2, prepared for dGH, hybridized with monochrome Ch 2 dGH paint. FIG. 4E shows a corresponding Chromosome 2 with complex structural rearrangements hybridized with monochrome Ch 2 dGH paint. The color channel map, shown in gray scale, for individual dGH bands (1-19) is provided in FIG. 10A.

FIG. 5A-FIG. 5D illustrate an example of Targeted Probe dGH Assays for SV detection. FIG. 5A shows normal Chromosome 2, prepared for dGH, hybridized with 4 targeted probes around a locus of interest. FIG. 5B shows chromosome 2 with deletion of portion of the locus of interest (spanning the genomic coordinates covered by targeted probe 2). FIG. 5C shows chromosome 2 with a sister chromatid recombination event, with targeted probes 2 and 3 seen on the opposite sister chromatid from targeted probes 1 and 4, with the order of the probes maintained—1, 2, 3, 4 from telomere to centromere. FIG. 5D shows chromosome 2 with an inversion event, where targeted probes 2 and 3 can be seen on the opposite sister chromatid from targeted probes 1 and 4, with the order of probes 2 and 3 reversed. Probes appear in 1, 3, 2, 4 order from telomere to centromere. The color channel map, shown in gray scale, for individual targeted dGH probes is provided in FIG. 10B.

FIG. 6A-FIG. 6B illustrate an example image of single color (monochrome) dGH paint labelling Chromosomes 1, 2, and 3 in a rearranged cell from a radiation exposed blood-derived lymphocyte sample prepared for dGH. FIG. 6A shows a karyogram of Chromosome 1, Chromosome 2 and Chromosome 3 homolog pairs (cropped and enlarged from metaphase spread image). FIG. 6B shows the entire original metaphase spread image.

FIG. 7A-FIG. 7D illustrate an example of using dGH banding to detect normal repair events in Chromosome 2 homolog pairs from BJ-5ta normal immortalized human fibroblast cell line. FIG. 7A and FIG. 7B show individual images of Ch 2 homolog pairs from two separate normal metaphase cells with no structural variation or repair event present. FIG. 7C and FIG. 7D provide individual images of Ch 2 homolog pairs from 2 separate metaphase cells. In each individual image, the chromosome on the left shows a normal repair event resulting from sister chromatid exchange (the order of the colors, shown in gray scale, is maintained, but the signals are present on the opposite sister chromatid).

FIG. 8A-FIG. 8I illustrate an example of using dGH banding to detect and define the location of an SCE in a chromosome 2 homolog pair from BJ-5ta normal immortalized human fibroblast cell line. FIG. 8A-FIG. 8D relate to the normal chromosome 2 sample, and FIG. 8E-FIG. 8F relate to the test chromosome 2 sample in which an SCE is present. FIG. 8I is an expanded image of both FIG. 8A and FIG. 8E, showing a G-banded ideogram of human chromosome 2 for genomic context. FIG. 8B shows an image overlay of the hybridization pattern of the dGH probes for normal chromosome 2. FIG. 8C shows the oligonucleotide distribution of the dGH probes (y axis) plotted along the length of the chromosome (x axis) for a normal chromosome 2. FIG. 8D shows the fluorescent wavelength intensities of the hybridized dGH probes of FIG. 8B for each sister chromatid, labeled Watson and Crick, of the normal chromosome 2 homolog, where the wavelength intensities for each color channel are overlayed. Labeled color channels, shown in gray scale, and indicated by line marks, include Dapi (square), Aqua (bold line), Green (line), TRITC (triangle), Red (diamond), and Cy5 (oval), as shown. FIG. 8F shows the hybridization pattern of dGH probes for a chromosome 2 with an SCE, where 810 shows the hybridization pattern of dGH probes on one chromatid of the chromosome 2, and the exchanged portion of the chromatid, originally hybridized with dGH probes (820), now on the sister chromatid. FIG. 8G shows the oligonucleotide distribution of the dGH probes (y axis) plotted along the length of the chromosome (x axis) for the chromosome with an SCE, and FIG. 8F shows the fluorescent wavelength intensities of the hybridized dGH probes of FIG. 8F for each sister chromatid, labeled Watson and Crick, for an SCE detected in one homolog of Chromosome 2, using the same color channels as described for FIG. 8D.

FIG. 9 illustrates 3 separate ladder assays hybridized to the chromosomes. One ladder measures limit of detection with respect to the number of oligos contributing to each signal, spaced roughly 20 mb apart on the p-arm of Chromosome 2 (labelled Ladder 1 in the image). A second ladder (Chromosome 2q) assesses the target size a fixed amount of oligos can be spread out over, also spaced about 20 MB apart, and also measures limit of detection (labelled Ladder 2 in the image). A third ladder (seen below hybridized to Chromosome 1q, has probes spaced close together as well as farther apart, allowing for an assessment of the resolvability two spots in close proximity in any given metaphase spread (labelled Ladder 3 in the image).

FIG. 10A Legend for color channels relevant to the banding pattern of chromosomes shown in FIG. 1A(i)-FIG. 1A(ii), FIG. 1C(i)-FIG. 1C(iii), FIG. 2A(i)-FIG. 2A(ii), FIG. 2C(i)-2C(ii), FIG. 3A(i)-FIG. 3A(ii), FIG. 3C(i)-FIG. 3C(ii), FIG. 4B, and FIG. 4C. Color channels for multicolor paint for dGH bands of sister chromatids corresponding to the hybridized dGH probes. For the listed figures, the legend shown in gray scale, shows bands 1, 3, and 5 are in the red color channel (A. Red). Bands 2, 4, 6, 13, 15 and 17 are in the green color channel (B. Green). Bands 7, 9, 11 and 19 are in the purple color channel (C. Purple), Bands 8, 10, and 12 are in the yellow color channel (D. Yellow), and bands 18, 16, and 14 are in the orange color channel (E. Orange), along the respective sister chromatids.

FIG. 10B Legend for color channels relevant to banding pattern of targeted sections of the respective sister chromatids in FIG. 5A to FIG. 5D. Targeted probe 1 is in the red color channel, labeled A. Red. Targeted probe 2 is in the green color channel, labeled B. Green. Targeted probe 3 is in the purple color channel, labeled C. Purple, and targeted probe 4 is in the orange color channel, labeled D. Orange.

FIG. 11 illustrates an example of whole genome dGH banding, showing a karyogram of dGH banded chromosomes from a metaphase spread of a diploid human cell. Chromosomes 1-22, and X are aligned with their homolog and numbered, as shown. To the left of each chromosome pair, an ideogram representing the specific dGH banding pattern for that chromosome is shown for genomic context. Chromosome Y is banded with only 1 dGH probe, as seen in the image.

FIG. 12 illustrates a workflow for an assay utilizing directional genomic hybridization.

FIG. 13 illustrates an exemplary workflows utilizing cell sorting, directional genomic hybridization, and an array, according to certain illustrative methods disclosed herein.

FIG. 14 illustrates a support matrix having 6 rows of partitions, each including a two-dimensional, regularly spaced arrangement of spots.

FIG. 15A is a cell cycle histogram showing DNA distribution of a dGH processed cell sample.

FIG. 15B is a scatter plot of dGH processed cells sorted by FACS after setting the gate based on the cell cycle histrogram of FIG. 15A.

FIG. 16A is a fluorescence image obtained after dGH analysis using a chromosome 1 dGH paint, of a DAPI stained control GM cell population showing the presence of desired cells.

FIG. 16B is a fluorescence image obtained after dGH analysis using a chromosome 1 dGH paint, of a DAPI stained control GM cell population showing the presence of non-desired cells.

FIG. 17 illustrates a two-dimensional, regularly spaced arrangement on a support matrix of 4 spots of cells in a left and right partition (top) and exemplary metaphase images (fluorescence images) for spots A1 (left-bottom panel labeled as “Metaphase from Spot A1”), and A3 (right-bottom panel labeled as “Metaphase from Spot A3”).

### DEFINITIONS

As used herein, “band” refers to a chromosomal region hybridized with a pool of fluorescently labeled, single-stranded oligonucleotides labeled with a similar light emission signature (e.g., pools of oligonucleotides of the same color).

As used herein, “bleeding” refers to the light emission signature of one band partially overlapping or otherwise partially appearing on at least one other band.

As used herein, “color” refers to the wavelength of light emission that can be detected as separate and distinct from other wavelengths.

As used herein, “consistent” refers to uniformity or an aspect of sameness. In certain aspects, an assay may utilize consistent hybridization probes indicating that the hybridization probes applied to two or more partitions are the same in sequence, label and/or other feature.

As used herein, “chromosome segment” refers to a region of DNA defined by start and end coordinates in a genome (e.g., bp 12900-14900 in Human Chromosome 2) or known sequence content (e.g., the sequence of a gene or mobile element). A chromosomal segment can be as small as a two base pairs, or as large as an entire chromosome.

As used herein, “color channel” refers to a region of the light spectrum, including visible light, infrared light and ultraviolet light. A color channel may be specified to be as broad a set of wavelengths or as narrow a set of wavelengths as useful to an individual practicing the methods disclosed herein.

As used herein, “directional genomic hybridization” or “dGH” refers to a method of sample preparation, such that the sister chromatids of a metaphase spread become single-stranded, combined with a method of hybridization with a dGH probe made up of a pool of single-stranded oligonucleotides before chromosome visualization using fluorescent microscopy. Further details regarding dGH and a dGH reaction are provided herein.

As used herein, “dGH probe refers to a pool of single-stranded oligonucleotides that comprise a same fluorescent label of a set of fluorescent labels, complementary to at least a portion of a target DNA sequence and wherein each of the dGH probes comprises at least one label.

As used herein, “enrich” refers to increasing the proportion of a component in a mixture. In certain aspects, enrichment of metaphase cells refers to increasing the proportion of cells in metaphase present in a population of cells.

As used herein, “episome” or “episomal DNA” refers to a segment of DNA that can exist and replicate autonomously in the cytoplasm of a cell.

As used herein, “extrachromosomal DNA” or “ECDNA” refers to any DNA that is found off the chromosomes, either inside or outside the nucleus of a cell. In certain aspects, ECDNA can be deleterious and can carry amplified oncogenes. In some aspects, deleterious ECDNA can be 100-1,000 times larger than kilobase size circular DNA found in healthy somatic tissues. In certain aspects, ECDNA includes episomal DNA and vector-incorporated DNA.

As used herein, “feature nodes” and “nodes” are used interchangeably to refer to numerical values, including sets of numerical values, representing any region of analytical interest on an oligonucleotide or polynucleotide strand. Nodes can be a specific locus, a string of loci, a gene, multiple genes, bands, or whole chromosomes. Nodes can be configurable and variable in size to allow different levels of granularity during analysis. By way of non-limiting example, nodes can represent normal features or abnormal features of a subject DNA strand. Also, by non-limiting example, nodes can provide numerical values for spectral profile data from labeled dGH probe hybridization to control DNA strands, where nodes represent either normal structural features or abnormal structural features of the control DNA strand.

As used herein, “feature lookup table” refers to a table of numerical values which represents one or more feature node.

As used herein, “grid” refers to a two-dimensional, regularly spaced arrangement of partitions such that lines connecting the partitions along an x-axis or along a y-axis form right angles at their intersection. Such grid can be formed on a support matrix.

As used herein, a “metaphase spread” is a set of metaphase chromosomes from a single cell's nucleus prepared on a sample support matrix such as a glass slide.

As used herein, “partition” refers to a structure or action which divides cells or chromosomes typically on a support matrix, such that some level of containment and/or separation is provided for the cells and/or chromosomes. While a partition can be provided by a physical barrier providing for some level of containment, partition can also be provided by a selected separation distance. In certain aspects, cells can be partitioned into separated groups of cells. In certain aspects, chromosomes can be partitioned into separated groups of chromosomes. In certain aspects, a partition may be a well of a multi-well plate. In certain aspects, cells and/or chromosomes can be partitioned at selected, separated locations on a microscope slide.

As used herein, “single stranded chromatid” refers to the product of the process in which a DNA analog (e.g BrdU/C) is provided to an actively dividing cell for a single replication cycle, which is then incorporated selectively into the newly synthesized daughter strand, a metaphase spread is prepared, the incorporated analog is targeted photolytically to achieve DNA nicks which are used to selectively to enzymatically digest and degrade the newly synthesized strand, resulting in a single-stranded product. If we use the terms Watson and Crick to describe the 5′ to 3′ strand and 3′ to 5′ strand of a double-stranded DNA complex, an untreated metaphase chromosome will have one sister chromatid with a parental Watson/daughter Crick, one sister chromatid with a daughter Watson/parental Crick. In the chromosomes prepared according to the method above, one sister chromatid will consist of the parental Watson strand only, and the other sister chromatid will consist of the parental Crick strand only.

As used herein, “sister chromatid exchange” or “SCE” refers to an error-free swapping (cross-over) of precisely matched and identical DNA strands. Sister chromatid exchanges, while not structural variants, are associated with elevated rates of genomic instability due to an increased probability that alternative template sites such as repetitive elements adjacent to the break site will produce an unequal exchange resulting in a structural variant.

As used herein, “sister chromatid recombination” or “SCR” refers to the homologous recombination process involving identical sister chromatids that results in a uni-directional non-crossover event, otherwise known as a gene conversion event. It is thought to occur when the homologous recombination intermediate known as the double Holliday junction is resolved in such a way that it results in a non-crossover. SCR can be employed by the cell to resolve both single-stranded DNA lesions (which involve a corresponding replication fork collapse) and double-stranded breaks. Gene conversion between sister chromatids is not usually associated with reciprocal exchange, and is differentiated from an SCE for that reason.

As used herein, “spectral profile” refers to the graphic representation of the variation of light intensity of a material or materials at one or more wavelengths. A material can be, for example, a chromosome or a single-stranded chromatid, or a region thereof.

As used herein, “stretched” refers to reducing the level of DNA compaction. In certain aspects, stretching of DNA refers to separation of DNA from the nucleus and DNA packaging proteins via protease digestion, releasing the DNA molecules into solution, and then stretching the chromosomes along a solid surface through a dewetting process. In other aspects stretching refers to using a pressure-driven microfluidic flow to extract and stretch chromosomal DNA from individually isolated cell nuclei immobilized in microchannels.

As used herein, “structural feature” refers broadly to any aspect of a sequence of bases within an oligonucleotide or polynucleotide, including normal features or abnormal features of a sequence. For example, structural features include but are not limited to genetic elements selected from a protein coding region, a region which affects transcription, a region which affects translation, a region which affects post-translational modification and any combination thereof. By way of further non-limiting example, structural features include genetic elements selected from an exon, an intron, a 5′ untranslated region, a 3′ untranslated region, a promotor, an enhancer, a silencer, an operator, a terminator, a Poly-A tail, an inverted terminal repeat, an mRNA stability element, and any combination thereof.

As used herein, “structural variant”, “structural variation”, “chromosomal structural variant” or “SV” refers to a region of DNA that has experienced a genomic alteration resulting in copy, structure, and content changes over 50 bp in segment size. The term SV used as an operational demarcation between single nucleotide variants/INDELs and segmental copy number variants. These changes include deletions, novel sequence insertions, mobile element insertions, tandem and interspersed segmental duplications, inversions, truncations and translocations in a test genome as it compares to a reference genome.

As used herein, “target DNA” refers to a region of DNA defined by start and end coordinates of a reference genome (e.g. bp 12900-14900 in Human Chromosome 2) or known sequence content (e.g., the sequence of a gene or mobile element) that is being detected.

As used herein, “target enrichment” refers to utilization of additional dGH probes, beyond those dGH probes used for banding, to a targeted area of interest, in order to track any changes to that specific region. In certain aspects, the targeted area of interest may be smaller than a band. In certain aspects, the targeted area of interest may be limited to a portion of a band, cover one whole band, or span across portions of or the entirety of two or more bands.

As used herein, “trained” or “training” refers to creation of a model which is trained on training data and can then be used to process addition data. Types of models which may be used for training include but are not limited to: artificial neural networks, decision trees, support vector machines, regression analysis, Bayesian networks, and genetic algorithms.

As used herein, “vector incorporated DNA” refers to any vectors which act as vehicles for a DNA insert. These may be cloning vectors, expression vectors or plasmid vectors introduced into the cell, including but not limited to artificial chromosome vectors, phage and phagemid vectors, shuttle vectors, and cosmid vectors.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 1 to 49, 1 to 25, 1.7 to 31.9, and so forth (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. In addition, each disclosed range includes up to 20% lower for the lower value of the range and up to 20% higher for the higher value of the range. For example, a disclosed range of 4-10 includes 3.2-12. This concept is captured in this document by the term “about”. When multiple low and multiple high values for ranges are given that overlap, a skilled artisan will recognize that a selected range will include a low value that is less than the high value.

As used herein, “about” or “consisting essentially of” mean±20% of the indicated range, value, or structure, unless otherwise indicated. As used herein, the terms “include” and “comprise” are open ended and are used synonymously. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. Aspects and embodiments illustratively described herein suitably may be practiced in the absence of any element or elements, limitation or limitations which is not specifically disclosed herein.

Although methods and materials similar or equivalent to those described herein can be used in the practice or testing of the present disclosure, suitable methods and materials are described below. All publications, patent applications, patents, and other references mentioned herein are incorporated by reference in their entireties. In case of conflict, the present specification, including explanations of terms, will control. In addition, the materials, methods, and examples are illustrative only and not intended to be limiting.

It is appreciated that certain features of aspects and embodiments herein, which are, for clarity, discussed in the context of separate embodiments, may also be provided in combination in a single embodiment. Conversely, various aspects and embodiments, which are, for brevity, discussed in the context of a single aspect or embodiment, may also be provided separately or in any suitable sub-combination. All combinations of aspects and embodiments are specifically embraced herein and are disclosed herein just as if each and every combination was individually and explicitly disclosed. In addition, all sub-combinations of the various aspects and embodiments and elements thereof are also specifically disclosed herein even if each and every such sub-combination is not individually and explicitly disclosed herein.

### DETAILED DESCRIPTION

The present disclosure addresses many long-felt needs and long-standing problems in the art. for example related to chromosomal imaging. Such aspects and embodiments in some embodiments, increase assay throughput for example by utilizing arrays to perform analysis that is traditionally performed in single reactions. Furthermore, certain illustrative embodiments, especially those that involve using dGH probes labeled with fluorescent labels such that 2 dGH probes have differently colored fluorescent labels and bind target DNA sequences on the same chromosome or chromatid, increase resolution compared to prior FISH and dGH methods. Furthermore, methods herein utilize cell enrichment, which in illustrative embodiments is accomplished using cell and/or chromosome sorting methods.

There are several types of benefits to cell enrichment that solve long-standing problems:

(1) Time/cost saving: Currently, dGH and FISH assays are typically performed on samples that yield between 10-500 metaphases per slide. Samples that yield fewer than 50 spreads per slide are often discarded due to the technical hurdles involved with hybridizing and analyzing 10 s or 100 s of slides. Discarded samples either need to be re-processed (in the case that more sample is available) or removed from the study. Re-processing may take from days to weeks to occur depending on how the sample was obtained. It is possible that even after re-processing, mitotic index will remain low.

(2) Efficiency: The number of slides needed to process multiple samples with a single assay OR multiple assays on a single sample can be drastically decreased using the cell enrichment methods disclosed herein.

(3) Ability to perform panels on different cell sub-populations within a larger population. There are several examples of why this type of analysis is useful in the context of FISH and in illustrative embodiments, dGH FISH. The following examples regarding panels on cell sub-populations are provided only for illustrative purposes and not intended to be limiting as there are other contexts where such panel approaches may be utilized.

(3)(A) Analysis of successfully edited vs. non-edited cells: If a gene editing group is interested in a comparison of rearrangements in successfully edited cells vs. cells that were not successfully edited within the same population, in the absence of a unique insert sequence the only way to achieve this is to enrich for each population and analyze them separately. There is no way to distinguish these populations using chromosome analysis of a mixed sample.

(3)(B) Analysis of T cell subsets: When T cells are activated, they may skew toward different lineages based on the type and strength of the stimulant (among other factors), resulting in various subsets. These subsets may include effector and memory subsets. These T cell subsets play unique roles in an immune response and can significantly impact disease outcome. T cell activation (and subsequent proliferation) is a required step for generation of metaphase spreads for T cells and is therefore well-suited for FISH, or in illustrative embodiments dGH preparation of T cells. When CAR-T cells are infused into patients, the desired outcome is enhanced effector and killing functions against the target population. By performing cell enrichment, we can gather data on the prevalence of rearrangements within different subsets which provides valuable information for downstream clinical applications.

(3)(C) Exclusion of feeder cells: Several cell types require co-culture with irradiated feeder cells to survive and/or expand, including NK cells. Although these irradiated feeder cells should not expand due irradiation induced block of mitosis, there is the potential for a small sub-population to survive this exposure and expand during co-culture. If this occurs, there is no way to distinguish the target cell population vs. the feeder cell population, which could severely compromise data since radiation exposure is known to cause genomic instability and rearrangements.

(3)(D) A combination of the foregoing approaches/needs may be applicable to a single situation.

(4) Gene Panel Array configuration allows for banded dGH assays or FISH reactions using sets of multicolor dGH probes, to be sub-divided for analysis. It also allows localized or targeted dGH probes to be used as a screening tool.

(5) Without enrichment of cells in metaphase, the dGH-based, and FISH-based array methods disclosed herein are less consistent. Non-enriched dGH and/or FISH preparations consist of both nuclei from cells in interphase and chromosome spreads from cells in metaphase. Usually, only about 10% or less of a non-enriched preparation is comprised of metaphase spreads. Preparations enriched for metaphase spreads can be spotted such that a more consistent number of enriched metaphase spreads per volume and per spot can be achieved across multiple samples/patients. Otherwise, each patient sample will be widely variable in terms of the number of spreads per spot. Although the spots in some embodiments will include both M and G2 cells (which will still result in some non-metaphase nuclei per spot), enrichment allows for more consistent and scalable spotting methods to be developed.

(6) Improved limit of detection (LOD) over other methods, such as array CGH. LOD of array CGH is typically 200-500 kb, whereas current dGH LOD can be as low as 3, 2, or 1 kb, which can be influenced by a number of factors including the density of unique target DNA sequences on a chromosome.

Aspects and embodiments herein, address the inability of sequence-based methods to be used in de novo measurement of structural variation in a chromosome. Further, the methods as disclosed herein assist in targeted measurements of known structural variations in a chromosome better than sequence-based methods. Finally, some illustrative aspects provide multi-color methods that are superior to monochrome methods at detecting and classifying chromosome structural features, such as structural variants, and chromosome repair events. The present disclosure relates generally to detection of structural features in chromosomes using fluorescent probes and fluorescence analysis. In illustrative embodiments, the structural features can include structural variations. In certain illustrative embodiments, methods disclosed herein can detect at least one repair event in a chromosome. Furthermore, in illustrative embodiments the methods as disclosed herein use chromosome-specific combinatorial labeling for detection of potentially deleterious structural variations, including but not limited to translocations amplifications, deletions, and inversions.

Accordingly, in one aspect, provided herein is a method for detecting a target DNA sequence in a chromosome of individual cells of a population of cells, the method comprising the steps of:
a) applying a cell sorting method to the population of cells to generate a sorted subpopulation of cells, wherein the cell sorting is based on a cell cycle stage, the presence of one or more target cell surface markers, the presence of one of one or more specific chromosomes, the presence of a target DNA sequence or a set thereof, or the presence of a structural feature on the chromosome,
b) contacting a pair of single-stranded sister chromatids in a metaphase spread prepared from individual cells of the metaphase-enriched cell population, with a first dGH probe, the first dGH probe comprising a first colored fluorescent label of a set of fluorescent labels, wherein the first dGH probe comprises a pool of single-stranded oligonucleotides that comprise a same fluorescent label of the set of fluorescent labels, wherein each single stranded oligonucleotide of a pool binds a different complementary DNA sequence within a same target DNA sequence found on one of the single-stranded sister chromatids;
c) detecting a fluorescence signal from a hybridization pattern of the first dGH probe to one or both single-stranded sister chromatids of the pair, thereby detecting the target DNA sequence in the chromosome.

In some embodiments, detecting the target DNA sequence, or a plurality of such target DNA sequences, can be used to detect a structure feature such as, for example, a structural variation, and/or to detect a repair event in the chromosome.

In another aspect provided herein is a method for detecting a target DNA sequence, a structural variation, and/or a repair event on a target chromosome from a population of cells, comprising:
(a) hybridizing a set of single-stranded sister chromatids comprising a chromatid derived from the target chromosome with a preliminary directional genomic hybridization (dGH) probe, wherein the preliminary dGH probe comprises a pool of single-stranded oligonucleotides complementary to a portion of a first target DNA sequence on one of the single-stranded sister chromatids or the single-stranded chromosomes, and comprising a preliminary fluorescent label;
(b) staining the single-stranded chromatids or the chromosomes obtained after the hybridizing of step (a) with a DNA stain to obtain a stained chromatid suspension or a stained chromosome suspension;
(c) applying a fluorescence sorting method on the stained chromatid suspension or the stained chromosome suspension to obtain an enriched chromatid suspension comprising a chromatid derived from the target chromosome or an enriched chromosome suspension comprising the target chromosome; (d) placing the chromatids from the enriched chromatid suspension into one or more partition of a two-dimensional spatial arrangement of partitions, or onto the same addressable position within each partition of a set of partitions;
(e) subjecting each of the chromatids placed into one or more partitions or the same addressable position to a fluorescence-based detection method comprising a first dGH probe comprising a first colored fluorescent label; and
(f) performing fluorescence analysis of the chromatids placed into one or more partitions or same addressable position, by detecting fluorescence signals generated based on a hybridization pattern of the first dGH probe to the single-stranded chromatids comprising a chromatid derived from the target chromosome to detect the target DNA sequence. In some embodiments, the method further comprises enumerating the structural features on the target chromosome from the population of cells. The enumerating can be, for example counting the number of target chromosomes that have the target DNA sequence, structural feature, and/or repair event, and/or the enumerating can be generating a numbered list of structural features identified on the target chromosomes. Thus, in some embodiments, this aspect is a method of counting the number of chromosomes, ratio of chromosomes, or percentage of chromosomes in a population that have a target sequence, structural variation, and/or repair event. In some embodiments, the structural feature comprises a structural variation on the target chromosome.

In some embodiments, the detecting detects a structural feature in at least one of the chromatids in at least one of the partitions or the same addressable position.

It is noteworthy that typically herein the terms chromosome and double-stranded chromatid can be used interchangeably.

### Cell Populations and Chromosomes

Cells and in illustrative embodiments, populations of cells are analyzed in methods provided herein, or are used for detecting at least one structural feature such as structural variation or repair event in a chromosome of individual cells from the population of cells. Accordingly, in some embodiments, the method may include assessing 1 or more than 1 cell, for example, at least 1, 2, 3, 4, 5, 6, 12, 15, 20, 30, 40, 50, 75, or 100 cells from a population of cells. For example, the method may include assessing between 2 and 20, 25, 30, 35, 40, 45, 50, 55, 60, 65, 70, 75, 80, 85, 90, 95, or 100 cells from the population of cells, or any number of cells there between the range. In other embodiments, the method includes assessing between 4 and 20, 25, 30, 35, 40, 45, 50, 55, 60, 65, 70, 75, 80, 85, 90, 95, or 100 cells from the population of cells. In other embodiments, the method includes assessing between 10 and 20, 25, 30, 35, 40, 45, 50, 55, 60, 65, 70, 75, 80, 85, 90, 95, or 100 cells from the population of cells. In other embodiments, the method includes assessing more than 100 cells from the population of cells. Some embodiments include assessing the same or a substantially similar number of cells from a control or reference cell population.

In some embodiments the population of cells can be a diploid population of cells. In other embodiments, the population of cells can be a complex population of cells having a complex karyotype. Cells with a complex karyotype do not have a wild-type number of chromosomes and have at least 2, 3, 4, 5, 6 or more chromosomal variants. Such diploid population can be a genetically-engineered population of mammalian cells, such as human, murine, canine, non-human primate and CHO cells. For example, a population of genetically modified blood cells, such as B cells, T cells, or NK cells, liver cells, kidney cells, iPSCs, or cells whose genome has been edited. In some embodiments, the population of cells are not genetically engineered. Complex cell populations are typically cancer cell populations. Examples of cancer cells include cells with 1Q deletions, translocations, bladder cancer, cells with translocations on the chromosomes, cells having chromosomes with random inversions, cells with high copy number of chromosomes such as numerical variants or non-normal numbers, cells with chromosomal structural variations, cells having genomes with mutations or single nucleotide polymorphisms (SNPs), cells having large inserts on the chromosomes, cells from different cancers/subsets, cells having chromatin condensation defects (detectable—long spindly centromeres), cells having chromatin remodeling and chromatin changes, or chromatin rearrangements, cells with PARPI knockout, cells from any known disease having any known genetic markers for genetic diseases such as cells having ALK-EML4 inversion, cells having BRCA1 (repair protein in breast cancer), cells having HER2 activation (which is initially virulence—then later a stability-driven cancer). Chromatin changes occur in response to DNA damage and involve histone modifications, chromatin remodeling, recruiting histone variants and histone chaperones. Chromatin modulation through PARylation initiates the DNA damage response and promotes DNA repair. PARylation is mediated by poly-(ADP-ribose) polymerases (PARPs). PARP1 transfers ADP-ribose from NAD- to protein acceptor sites. Increased PARP1 activity leads to reduced NAD+ levels and cell death. The activation of PARP1 and accumulation of PAR have been demonstrated in post-mortem brains of Alzheimer's disease patients, particularly in neurons of the frontal and temporal lobes. Together, these observations indicate that chromatin modifications play a major role after DNA damage in Alzheimer's disease and suggest that interventions aimed at inhibiting chromatin modification such as PARP1 inhibition, may slow down the progression of Alzheimer's disease.

In some embodiments, the population of cells is a population of control or reference cells, such as but not limited to a population of T cells from a healthy subject, or a population of cells from an immortalized T cell line whose chromosome stability value or score is known/has been determined. In some embodiments, the population of control or reference cells is a population of cells of a cancer cell line with a known/predetermined chromosome stability score. In some embodiments, such as but not limited to when a genetically-modified population of cells is being analyzed, the population of reference or control cells is a population of cells from the same type of cell or descendants of the same cells that are not genetically modified, as those that were genetically modified. In some embodiments, two or more populations of cells that are of the same cell type and in illustrative embodiments, descendants of the same cell population, are analyzed together. In some embodiments of methods herein, one or more cells of a population of cells are derived from cell culture. In some embodiments, one or more cells of a population of cells are derived from a tissue sample.

In some embodiments, one or more cells of a population of cells express at least one reporter protein, for example a fluorescent reporter protein, or a reporter protein that is a binding pair member with a fluorescent protein or an antibody, which in turn can be fluorescent or bound by a fluorescent secondary antibody. In some embodiments, methods herein further include sorting of cells based on the expression of at least one reported protein. In some embodiments, one or more cells of a population of cells can express two, three, four, five, or more different types of reporter protein. A skilled artisan can exploit the ability of any such reporter protein known in the art that can be used to sort cells of a population.

In some embodiments, one or more cells of a population of cells express at least one type of cell surface markers. In some embodiments, the cell surface markers can be selected from the group consisting of CD3, CD4, CD8, CD4RA, and combinations thereof. In some embodiments, one or more cells of a population of cells can express CD3 cell surface marker. In some embodiments, one or more cells of a population of cells can express CD4 cell surface marker. In some embodiments, one or more cells of a population of cells can express CD8 cell surface marker. In some embodiments, one or more cells of a population of cells can express CD4RA cell surface marker. In some embodiments, one or more cells of a population of cells can express any cell surface marker known in the art that can be used to sort the population of cells as described in methods herein.

CAR-T cell therapy is a type of treatment in which a patient's T cells (a type of immune system cell) are changed in the laboratory so they will attack cancer cells. T cells are taken from a patient's blood. Then the gene for a special receptor that binds to a certain protein on the patient's cancer cells is added to the T cells in the laboratory. The special receptor is called a chimeric antigen receptor (CAR). Large numbers of the CAR T cells are grown in the laboratory and given to the patient by infusion. CAR T-cell therapy is used to treat certain blood cancers, and it is being studied in the treatment of other types of cancer. Also called chimeric antigen receptor T-cell therapy. CAR-T cells are T cells which are genetically modified such that they express a chimeric antigen receptor (CAR) that is specific for a predetermined antigen.

In some embodiments, the populations of cells is a population of induced pluripotent stem cells (iPSC). In some embodiments, the iPSCs are derived from a patient with an idiopathic neurodegenerative disease. In some embodiments, the iPSCs from the patient are tested on a neurological disease screening panel.

In some embodiments, the population of cells has any genetic insertion by means of lentiviruses, transposons, or adenovirus associated vector (AAV).

In some embodiments, the population of cells is from a Downs syndrome patient, such as having trisomy 21, or a Downs syndrome patient developing Alzheimer's, which indicates that genotoxicity is happening in the Downs syndrome patient. Some embodiments have population of cells from subjects with autism spectrum. Some embodiments have populations of cells from subjects having rare diseases, patients undergoing gene therapy treatment for muscle wasting disorders, or patients having any condition that leads to genotoxicity. Populations of cells can be from neurodegenerative disease including schizophrenia, Alzheimer's, autism, and epilepsy.

In some embodiments, populations of cells exhibit mosaicism in blood or cell cultures, which shows as heterogeneous chromosomes. For example, an irradiated person treated with stem cells, then blood has many genomes-heterogeneous cell population; or in cases of clonal outgrowth where a few take over and faster and after a few rounds of multiplication the cells lose heterogeneity. Significant rearrangements which would trigger a higher genotoxicity risk score for these diseases include the following genes and loci, such as, somatic mosaicism, deletions, copy number variation or other structural rearrangements involving 2q31.2 (PRKRA gene), 5q35.2 (BOD1 gene) and 7p15.2 (CBX3 gene) in brain cells, and somatic mosaicism, deletions, copy number variation, or other structural rearrangements involving the NEGRI1, PTBP2, CADPS, KMT2, E KCNN2, MACROD2, MMP12, NTM, ANTXRL, CHST9, DNM3, NDST3, SDK1, STRC, SKY, SCN1A, SCN2A, SETD2, ARID1B, AKT1, AKT3, MTOR, PIK3CA, TSC1, TSC2, mTOR, PI3K-Akt, p53, and PTEN genes (various chromosomal loci), and a cluster of genes on Chr17 (KANSL1, WNT3, MAPT and CRHR1) in blood and neuronal cells.

Populations of cells include cells for instability testing where marker for instability are tested such as general genome hyperploidy, aneuploidy at chromosome 21, aneuploidy chromosome X, aneuploidy chromosome 18 and presence of micronuclei.

Genome editing, or genome engineering, or gene editing, is a type of genetic engineering in which DNA is inserted, deleted, modified or replaced in the genome of a living organism. Unlike early genetic engineering techniques that randomly inserts genetic material into a host genome, genome editing targets the insertions to site specific locations.

CRISPR (an acronym for clustered regularly interspaced short palindromic repeats) is a family of DNA sequences found in the genomes of prokaryotic organisms such as bacteria and archaea. Cas9 (or “CRISPR-associated protein 9”) is an enzyme that uses CRISPR sequences as a guide to recognize and cleave specific strands of DNA that are complementary to the CRISPR sequence. Cas9 enzymes together with a guide RNA CRISPR sequence form the basis of a technology known as CRISPR-Cas9 that can be used to edit genes within organisms.

Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, so when combined with a nuclease, DNA can be cut at specific locations.

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to alter the genomes of higher organisms.

Certain aspects of methods herein can include chromosomes derived from the same type of population of cells or same cell sample as described herein. In some embodiments, methods herein can include chromosomes from more than one, two, three, four, or five type of population of cells or cell samples. In some embodiments, methods herein can include chromosomes derived from cell culture. In some embodiments, methods herein can include any type of cells that can be cultured in a standard cell culture conditions known in the art. In some embodiments, methods herein can include chromosomes derived from a tissue sample. Type of tissue samples that can be used in methods herein, can include, but not limiting to, blood, bone marrow, and amniotic fluid. In some embodiments, methods herein can include chromosomes from a non-enriched cell population, for example, a cell population that is not subjected to any enrichment step. In some embodiments, methods herein can include chromosomes from an enriched cell population, in illustrative embodiments, metaphase-enriched cell population.

### Enrichment and Sorting

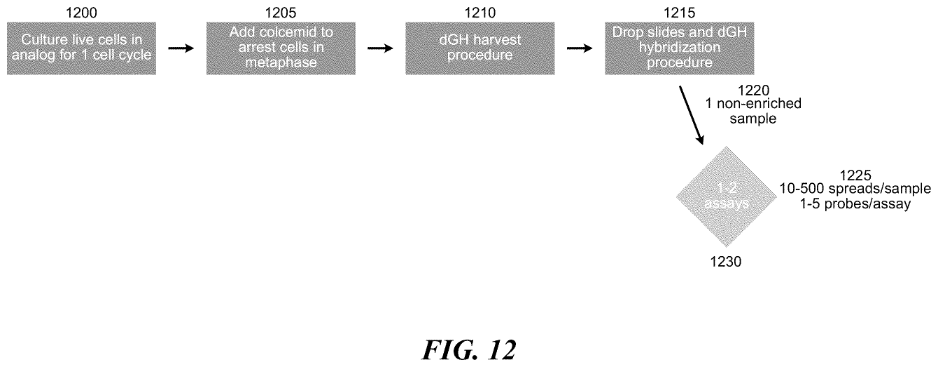

In certain aspects, methods are disclosed herein to increase the proportion of cells (i.e. enrich cells) in a cell population for cells that are in the metaphase stage of the cell cycle. This can be accomplished for example, by culturing (i.e., incubating) cells in a metaphase-arresting agent such as demecolcine, colchicine or N-methyl-N-deacetyl-colchicine (Colcemid™). In some embodiments, a thymidine block can be included during metaphase arresting stage. A non-limiting exemplary workflow for performing certain embodiments herein that include a treatment with a metaphase-arresting agent, is shown in FIG. 12. This can also be accomplished in part by staining chromosomes and sorting on cells with increased chromosome copy by using a DNA stain such as, for example, Hoechst 33258. It will be understood that such enriching for a subpopulation of cells in a metaphase stage can be accomplished by enriching/sorting for cells in the G2/M phase of the cell cycle. Cell enrichment can also include sorting for subpopulations of cells, including different cell types, e.g, blood cell types such as T cell types and/or edited vs. unedited cells. The metaphase-enriched cell population can then be used in methods provided herein for analyzing cells. In illustrative aspects, the metaphase enriched cell population can be used to create cell arrays on surfaces, for example flat, transparent surfaces, such as on microscope slides. Such surfaces can be relatively thin, compared to their length or width.

Many types of cell sorting methods are compatible with the methods disclosed herein including but not limited to any that use centrifugation, filtration, immobilization (e.g., microchip), fluorescence, or bead (e.g., magnetic) based methods, and in some embodiments such methods are automated methods (e.g., Bio-Rad S3e™). Automated methods can utilize a robotic liquid-handling platform, for example that is equipped to handle 96-well and/or 384-well plates (e.g., Bravo Automated Liquid Handling Platform (Agilent Technologies)) and an automated cell sorter can be utilized as well. After the cells are sorted, the cells can then be processed, in illustrative embodiments, by a dGH harvest protocol which, in some non-limiting embodiments include incubating the cells with a hypotonic solution typically to make the cells soft so that they break once the cells are dropped, placed, or deposited on slides which in some non-limiting embodiments, can be in the form of arrays. In some non-limiting embodiments, after the incubation with a hypotonic solution, the cells are fixed by exposing the cells to a fixative, which in illustrative embodiments, include methanol and acetic acid in a 3:1 volume ratio, to form processed cells or fixed cells. In some non-limiting embodiments, the fixed cells can be dropped, spotted, placed or deposited on slides or arrays. In some non-limiting embodiments, the cells are lysed and the chromatids from the lysed cells can optionally be spotted on arrays.

High throughput flow cytometric methods are powerful approaches for rapid, high volume analysis and/or sorting of individual cells or chromosomes based on multiplexed fluorescence intensity. Combining high throughput flow cytometry methods with dGH assay techniques allows for rapid identification of, and/or sorting based on structural features of interest on one or more chromosomes of interest. In embodiments disclosed herein, these high throughput methods can be used to rapidly detect a known rare structural variation or repair event, in some embodiments at a known genomic location from a large population of cells. In some embodiments, high throughput methods can be used to identify rare events at an unknown genome location from a population of cells. The population of cells can be from one individual, different individuals, or from cultured cells.

The cell sorting procedure performed in certain methods herein depends on the type of sorting system chosen and the specific embodiment being performed. In some embodiments, cell sorting is based on FACS cell surface staining. Different subpopulations can be identified using a cell surface marker, or a panel of cell surface markers, optionally plus DNA staining. Other sorting approaches may require different sample preparations. In some embodiments, cell surface staining is not performed. In such embodiments, the population of cells being sorted can be of the same type or lineage.

Some embodiments include a step of enriching metaphase cells in a population of cells, for example using colchicine exposure/treatment of the population cells. The number of cells enriched for metaphase depends on the type of cell as well as pretreatment of the cell. In some embodiments, as a non-limiting example using colchicine treatment, enriched metaphase cells can range from 1% to 30% or 1% to 25% of the cell population in some embodiments, or can increase the proportion of cells in a cell population that are in metaphase at a level that is a detectable increase in metaphase cells (e.g. a 10%, 20%, or 25% increase). It will be understood that the number of cells in metaphase in a population depends on cell type and how rapidly cells are dividing.

In some embodiments, the chromosomes of interest from enriched cell populations can be further sorted for analysis. In such embodiments, chromosomes can be directly sorted into tubes or partitioned onto slides or arrays for further analysis and testing.

In certain aspects, methods are disclosed herein to increase the proportion of cells in a cell population for cells that are in the metaphase stage of the cell cycle. Cell enrichment may also include sorting for subpopulations of cells, including different cell types, e.g, blood cell types such as T cell types and/or edited vs. unedited cells. The metaphase-enriched cell population can then be used in methods provided herein for analyzing cells. In illustrative aspects, the metaphase enriched cell population can be used to create cell arrays on surfaces, for example flat, relatively thin transparent surfaces, such as on microscope slides. In some embodiments, the chromosomes of interest can be sorted without sorting the cells in a prior step. In some embodiments, an un-sorted population of cells can be subjected to a treatment that comprises lysing the cells to isolate chromosomes, followed by labeling of the chromosomes or chromatids derived from the chromosomes, and sorting the chromosomes or chromatids for further analysis. In such embodiments, the sorting is done directly at the level of chromosomes after lysing the cells. In some embodiments, the sorting can be done by applying a fluorescence sorting method to obtain an enriched chromatid suspension. In some embodiments, the fluorescence sorting method applied on the chromosomes can comprise automated sorting method. In some embodiments, the un-sorted population of cells can undergo any of the cell sorting steps disclosed herein to provide an enriched cell population. In some embodiments, the enriched cell population can be subjected to a treatment that comprises lysing the cells to isolate chromosomes, followed by labeling of the chromosomes or chromatids derived from the chromosomes, and sorting the chromosomes or chromatids for further analysis.

In one aspect, live cells are cultured in the presence of BrdU/C analog nucleotides for one cell cycle and treated with a mitotic block (colcemid). Cells are then collected and labeled with a DNA stain (e.g., propidium iodide, 7-AAD, or Hoechst) after colcemid incubation. A staining panel for surface markers and/or GFP or other fluorescent marker expression can also be included. For example, a cell population which has been transduced with a GFP-tagged insert can be sorted based on GFP fluorescence and the presence of a DNA stain. Cells can be sorted, in illustrative embodiments, based on 4N (or more) DNA staining along with forward/side scatter and any other desired markers. As a non-limiting example Sergio J. Ochatt (2006) (Medicago truncatula handbook, Flow cytometry (ploidy determination, cell cycle analysis, DNA content per nucleus) (incorporated by reference herein in its entirety)) provides an example of how cells in different phases of the cell cycle can be identified based on DNA staining characteristics. Using cell sorting, cells within a metaphase-arrested cell population can be isolated that have a G2 DNA content, to produce a metaphase-enriched cell population.

In a further aspect, a sorting strategy can involve BrdU plus Hoechst staining (similar to the strategy laid out in Sanders A D, et al. Nat Protoc. 2017 June; 12(6):1151-1176. doi: 10.1038/nprot.2017.029, incorporated by reference herein in its entirety). BrdU incorporation is already a step in the dGH process, so using this sorting strategy should not disrupt sample processing or dGH signal. As cells divide, they preferentially incorporate BrdU instead of thymidine (T) into nascent DNA strands. When BrdU incorporated DNA is stained with Hoechst, a DNA intercalating dye that preferentially binds to double-stranded DNA at A-T sites, the bromine group of BrdU will quench the emission energy of nearby Hoechst. This results in staining intensity that is lower than non-dividing cells without BrdU incorporation. Metaphase cells can then be sorted and isolated based on Hoechst staining and isolating cells using the combination of Hoechst staining and BrdU ((Sanders et al. 2017) to produce a metaphase-enriched cell population. This approach can be combined with surface antibody staining panels and/or marker expression such as GFP. Thus, in certain embodiments, single-stranded sister chromatids (i.e. each single-stranded sister chromatid) are prepared, for example after a single cell division and/or from the metaphase-enriched cell population, by degrading chromosome strands (e.g. from double-stranded chromatids). The degrading can be performed by incorporating a DNA analog into genomic DNA of individual cells of a population of cells for one cell cycle, and degrading the newly synthesized chromosome strand (i.e. chromatid strand) that incorporated the DNA analog, which in some embodiments is a uridine and/or a cytidine analog, for example BrdU/BrdC. In some subembodiments of methods that comprise incorporation of a uriding analog, the method further comprises staining the single-stranded sister chromatids with a DNA stain, which in illustrative subembodiments is an intercalating dye that preferentially or exclusively binds to double-stranded DNA at A-T sites. Such a DNA stain in some embodiments is a bis-benzimide stain, such as a Hoechst stain (e.g. Hoechst 33258 or Hoechst 33342). A similar strategy is used in some embodiments BrdC and a DNA stain that binds preferentially or exclusively to G-C sites, or a combination of Brd and BrdC and a combination of DNA stains the preferentially or exclusively bind A-T sites and to G-C sites, or a DNA stain that binds indiscriminately.

In these non-limiting specific embodiments that include sorting based on a nucleotide derivative (BrdU) and DNA staining (Hoechst staining), a control sample (e.g., BrdU−) can be included to identify the undivided population and to calibrate the FACS system. Because these cells have not been exposed to the nucleotide analog (e.g, BrdU), they will exhibit ‘full’ Hoechst fluorescence. The stained cells can then be run through a FACS machine, and single cells can be gated, using size to discriminate doublets. When isolating cells by FACS, in illustrative embodiments, at least 10,000 cells are sorted to visualize a stable population in a gating plots. Furthermore, in some embodiments at least 5×10⁵ cells can be collected for all samples to be analyzed on a cell sorter. Since Hoechst can be excited with a UV (350-nm) laser, in non-limiting examples, a 488-nm detector can be used to record the emission. The nucleotide analog negative (e.g., BrdU−) control population can be visualized on a linear histogram and in illustrative embodiments is placed in the right-hand quadrant of a FACS plotFIG. The cell population that underwent one cell division in the presence of the nucleotide analog (e.g. BrdU) can be identified as the peak showing substantially less Hoechst fluorescence as compared with that of control cells that did not incorporate BrdU. Optionally, cells can be counterstained with propidium iodide (PI), to identify cell cycle stage and gate on cells in G2/M phaseFIG. In either case, in certain embodiments a narrow gate can be set to directly deposit the single-sorted cells onto an array. During the sort, library preparation controls can be introduced by sorting no cells (negative control) or multiple cells (e.g., 10, as positive control) into different positions on an array.

Thus, in some embodiments, cells that undergo a single division in the presence of a nucleotide analog are sorted based on (a) Hoechst fluorescence and, optionally, (b) a living or dead cell dye (e.g. fluorescein diacetate (FDA) or propidium iodide (PI)). FACS gates can be first set to select cells or nuclei (for example if sequencing is to be performed along with dGH) based on forward (FSC) and side (SSC) scatter, and doublets can be avoided by size. A negative-control sample (0 BrdU) can be used to set up laser voltages, sorting gates and to establish a Hoechst profile (visualized to a linear scale) for the undivided population. During calibration, a narrow gate (gate ‘x’) can be set on the Hoechst channel to establish the mean fluorescence for this population (table). During calibration the Hoechst peak can be placed in the right-hand half of a visual graphed plot of cell sorting gates based on Hoechst staining or Hoechst quenching in the above-identified embodiment, to allow room for the quenched population. The sorting gate (gate ‘y’) can be set on the peak showing ˜½ Hoechst fluorescence, compared with the undivided (0 BrdU) control. The percentage of cells that occupy this peak would increase with BrdU exposure, indicating that cells have undergone mitosis and taken up BrdU. In some embodiments, daughter cells arising from a single cell division (undergone mitosis) in the presence of BrdU contain hemi-substituted DNA which means that the nascent DNA strand has taken up or incorporated BrdU, whereas the original DNA template strand has not taken up or not incorporated BrdU. A plot of Hoechst area against PI or FDA area can be used to distinguish cell cycle stage based on FDA metabolite or PI fluorescence (which is not affected by BrdU). Bivariate fluorescence plots comparing Hoechst against FDA metabolite or PI intensity for a mixture of cells cultured with and without BrdU allow identification of cells suitable for dGH (and/or Strand-seq library construction (see below)). Cells in a target sorting region would have lower Hoechst fluorescence as compared with cells in another region of the plot, and would show fluorescence reflecting 4N DNA content (i.e., G2/M phase), in some embodiments, such cells can be hemi-substituted G2/M cell population. Cells in the target sorting region with quenched Hoechst fluorescence signal can be sorted and isolated as single cells, and in illustrative embodiments, can be distributed on an array for dGH analysis.

Such BrdU plus Hoechst sorting method is compatible for use with single cell strand-specific sequencing methods such as Strand-Seq (Sanders et al. 2017 (Single-cell template strand sequencing by Strand-seq enables the characterization of individual homologs. Nat Protoc. 2017 June; 12(6):1151-1176. doi: 10.1038/nprot.2017.029, incorporated herein by reference)). The sorting method plus a single cell, strand-specific sequencing method can be combined with dGH to provide a powerful analysis pipeline. In these embodiments, the cell sorting method can be used to sort a first subpopulation of cells from a population of cells using a G2/M gate to sort cells to be analyzed using a dGH reaction, and to sort a second subpopulation of cells from the population of cells, or nuclei therefrom, using a G1 gate to sort cells or nuclei therefrom, to be analyzed by sequencing. Thus, a first population of sorted cells can be analyzed using dGH probes, for example in a dGH assay, after a sorting performed using a G2/M gate, and a second population of cells can be analyzed by sequencing, for example in a strand-seq sequencing analysis, after sorting using a G1 gate.

Accordingly, in some embodiments, at least one nucleic acid is collected or isolated and sequenced. In some embodiments, the at least one nucleic acid is a DNA or RNA. In some embodiments, the method further comprises sequencing nucleic acids from the population of sorted cells. In some embodiments, the method further comprises collecting (or isolating) single-stranded sister chromatids from the sorted cell population, or separately from individual cells of the sorted cell population, and sequencing at least one, and typically a plurality of nucleic acids generated from the collected single-stranded sister chromatids. In some embodiments, of any aspects or embodiments herein that comprise nucleic acid sequencing, the nucleic acid sequencing is single-cell template strand sequencing, single molecule sequencing, long-read sequencing, and/or next-generation sequencing (NGS). Such sequencing methods can include one or more nucleic acid modification step (e.g. end-repair, A-tailing, and/or adapter (e.g. Y/forked adapter) ligation steps on nucleic acids to be sequenced, such as those isolated from a population of cells, as non-limiting examples, single-stranded sister chromatids therein. Such methods can optionally include one or more amplification steps of nucleic acids generated from the single-stranded sister chromatids, before performing an NGS reaction. Adapters that are ligated, and/or primers that are used in the one or more amplifications, can include NGS flow cell primer binding sites, sequencing primer binding sites, sample barcodes/indices, and/or molecular barcodes. In certain embodiments, to preserve directionality of template strands, genomic preamplification is bypassed (i.e. not performed) or performed after BrdU containing strand digestion, and labeled nascent, single-stranded sister chromatid strands are nicked and not amplified during library preparation. Thus a step of nicking single-stranded sister chromatids can be included in methods herein, for example those that include a sequencing step. Each single-cell library can be multiplexed (e.g. by arraying cells/nuclei and/or by using sample barcoding for example for each array spot, each cell and/or each array partition) for pooling and sequencing, and the resulting sequence data can be aligned, mapping to either the minus or plus strand of a reference genome, to assign template strand states for each chromosome in the cell.

As indicated above, the sorting method plus a sequencing method such as, but not limited to, single cell, strand-specific sequencing method can be combined with a dGH analysis to provide a powerful analysis pipeline. Thus, in one aspect provided herein is a method for analyzing one or more chromosomes, or analyzing structural features of one or more chromosomes, the method comprising the steps of:
a) contacting a pair of single-stranded sister chromatids in a metaphase spread prepared from individual cells of a population of cells, in illustrative embodiments a G2/metaphase or metaphase-enriched subpopulation from the population of cells, with a first dGH probe, the first dGH probe comprising a first colored fluorescent label of a set of fluorescent labels, wherein the first dGH probe comprises a pool of single-stranded oligonucleotides that comprise a same fluorescent label of the set of fluorescent labels, and wherein each single stranded oligonucleotide of a pool binds a different complementary DNA sequence within a same target DNA sequence found on one of the single-stranded sister chromatids;
b) detecting a fluorescence signal from the first colored fluorescent label, from one or both single-stranded sister chromatids;
c) collecting (or isolating) one or more, typically a population of single-stranded sister chromatids from the cell population, in illustrative embodiments a G1-enriched subpopulation from the population of cells, for example separately from individual cells or nuclei therein, of the cell population; and
d) sequencing the collected one or more, or typically the population of, single-stranded sister chromatids.

Accordingly, in another aspect, provided herein is a method for detecting at least one target DNA sequence, at least one structural variation and/or at least repair event in a chromosome of individual cells of a population of cells, the method comprising the steps of:
a) contacting a pair of single-stranded sister chromatids in a metaphase spread prepared from individual cells of a G2/metaphase, or metaphase-enriched subpopulation of a population of cells, with a first dGH probe, the first dGH probe comprising a first colored fluorescent label of a set of fluorescent labels, wherein the first dGH probe comprises a pool of single-stranded oligonucleotides that comprise a same fluorescent label of the set of fluorescent labels, wherein each single stranded oligonucleotide of a pool binds a different complementary DNA sequence within a same target DNA sequence found on one of the single-stranded sister chromatids;
b) generating a fluorescence pattern from one or both single-stranded sister chromatids using fluorescence detection, wherein the fluorescence pattern is based on a hybridization pattern of the first dGH probe to one or both single-stranded sister chromatids of the pair;
c) collecting (or isolating) one or more, typically a population of single-stranded sister chromatids from a G1-enriched subpopulation of the population of cells, in illustrative embodiments separately for individual cells of the cell population;
d) sequencing the collected one or more, or typically the population of, single-stranded sister chromatids from the G1-enriched subpopulation; and
e) detecting based on the fluorescence pattern and/or based on the sequencing, the presence of the at least one structural variation in the chromosome from individual cells. In some illustrative embodiments, the detecting is based on both the fluorescence pattern and the sequencing.

In these aspects that include both generating a fluorescence pattern and sequencing, the sorting is typically performed using fluorescence-based sorting. Such fluorescence-based sorting can include separately isolating the G2/M or metaphase-enriched subpopulation using a G2/M fluorescence gate and the G1-enriched subpopulation using a G1 fluorescence gate. The sequencing in illustrative embodiments, includes placing or depositing 10 or less cells, or nuclei therefrom, of the G1-enriched subpopulation in a two-dimensional spatial arrangement, such as in a multi-well plate or in a flat array as disclosed herein.

In illustrative embodiments of these aspects that include both generating a fluorescence pattern and sequencing, single-stranded sister chromatids (i.e. each single-stranded sister chromatid) are prepared, for example after a single cell division and/or from a G2/M or metaphase-enriched cell subpopulation and a G1-enriched cell subpopulation, by degrading chromosome strands (e.g. from double-stranded chromatids). The degrading can be performed by incorporating a DNA analog into genomic DNA of individual cells of a population of cells for one cell cycle, and degrading the newly synthesized chromosome strand (i.e. hemi-substituted chromatid strand) that incorporated the DNA analog, which in some embodiments is a uridine and/or a cytidine analog, for example BrdU/BrdC. In some subembodiments of methods that comprise incorporation of a uridine analog, the method further comprises staining the single-stranded sister chromatids with a DNA stain, which in illustrative subembodiments is an intercalating dye that preferentially or exclusively binds to double-stranded DNA at A-T sites. Such a DNA stain in some embodiments is a bis-benzimide stain, such as a Hoechst stain (e.g. Hoechst 33258 or Hoechst 33342). Furthermore, in some embodiments, such aspects include preparing a control population that is not incubated with the nucleotide analog.

In some embodiments, cell sorting methods can include sorting of cells based on at least one cell surface expression marker. In some embodiments, sorting of cells can include sorting based on CD3 expressed marker. In some embodiments, sorting of cells can include sorting based on CD4 expressed marker. In some embodiments, sorting of cells can include sorting based on CD8 expressed marker. In some embodiments, sorting of cells can include sorting based on CD4RA expressed marker. In some embodiments, sorting of cells can include sorting based on CD45RA expressed marker. In some embodiments, sorting of cells can include sorting based on CD197 expressed marker. In some embodiments, sorting of cells can include sorting based on any expressed marker known in the art that can be used in sorting of cells.

In some embodiments, methods herein can include cell sorting methods based on live/dead cell staining. In illustrative embodiments, live/dead cell staining include fluorescence-based live/dead cell staining. Fluorescence-based live/dead cell staining, in some embodiments, includes a simultaneous use of at least two fluorescent dyes that allows a two-color discrimination of a population of living cells from dead cell population. One non-limiting example of a fluorescence-based live/dead cell staining includes a staining protocol using fluorescein diacetate (FDA) and propidium iodide (PI), which stain viable cells and dead cells, respectively. In principle, FDA is taken up by cells which convert the non-fluorescent FDA into the green fluorescent metabolite fluorescein. The measured signal serves as indicator for viable cells, as the conversion is esterase dependent. In contrast, the nuclei staining dye PI cannot pass through a viable cell membrane. It reaches the nucleus by passing through disordered areas of dead cell membranes, and intercalates with the DNA double helix of the cell and can be seen in red color.

Methods herein, in some embodiments, can include sorting of chromosomes (chromosome sorting) from cells, in illustrative embodiments, metaphase-enriched cells. In some non-limiting exemplary workflow including chromosome sorting, includes culturing cells in the presence of colchicine to block the cells at metaphase, to produce a metaphase-enriched cell population. The enriched cell population are harvested, wherein the cells are lysed, in illustrative embodiments, by suspending in a hypotonic solution, which in some non-limiting embodiments can be a hypotonic KCl buffer ranging from 40 to 100, 50 to 90, or in illustrative embodiments, 60 to 85 mM KCl, wherein the cells are suspended for at least 3, 4, 5, 6, 7, 8, 9, in illustrative embodiments, 10 minutes. The cells are further suspended in a buffer, which in some illustrative embodiments, can be a chromosome isolation buffer comprising stabilizing agent or agents such as divalent cations, in illustrative embodiments, magnesium ions, and cationic polyamines, in illustrative embodiments, spermine, and spermidine to generate a chromosome preparation. In some non-limiting embodiments, the chromosome preparation are subjected to a preliminary probe hybridization which, in illustrative embodiments, include contacting the chromosome preparation with one or more oligonucleotide probes, in illustrative embodiments, one or more dGH probes, each probe comprising a pool of single stranded oligonucleotides, wherein each single stranded oligonucleotide of a pool binds a different complementary DNA sequence within a same target DNA sequence found on a specific chromosome from the chromosome preparation. In some non-limiting embodiments, each single stranded oligonucleotide of a pool binds a different complementary DNA sequence within a same target DNA sequence found on a specific chromosome that can be human chromosome 1, 2, 3, 4, 5, 6, 7, 8, 9, 10, 11, 12, 13, 14, 15, 16, 17, 18, 19, 20, 21, 22, X, or Y. In illustrative embodiments, each single stranded oligonucleotide of a pool binds a different complementary DNA sequence within a same target DNA sequence found on human chromosome 8, wherein, in some non-limiting embodiments, the target DNA sequence encompasses virtually the entire chromosome 8. After the hybridization step, the chromosomes are stained with a DNA stain, which in illustrative embodiments, can include Hoechst stain. The chromosomes are then subject to a sorting step, which in some non-limiting embodiments, include sorting of a specific chromosome from other chromosomes by flow cytometry, to obtain a sorted chromosome preparation. In some non-limiting embodiments, the sorted chromosome preparation can be analyzed such that chromosomes containing material specific to a specific chromosome, in illustrative embodiments, chromosome 8 would be represented in a karyotype.

Any dGH probe(s) that binds to a target DNA sequence that encompasses substantially an entire chromosome could be used to label chromosomes and to selectively sort against by this method. Sort gates can be set up so that chromosomes of variable size which contain the probe target sequence can be captured and isolated for downstream analysis including but not limited to dGH. In certain embodiments, hybridization of isolated chromosomes for the purposes of flow sorting would not go through dGH pretreatments (BrdU labeling, UV nicking and exonucleatic degradation of newly synthesized strand). In some embodiments, further analysis by dGH would require cells to be pre-treated with a nucleotide analog (e.g. BrdU) and sorted, or unsorted chromosomes to be fixed to a slide and subjected to such pretreatments (UV nicking and exonucleatic degradation of newly synthesized strand) before hybridization with one or more, in illustrative embodiments, two or more dGH probes. Any preliminary probe hybridization would not interfere with this process as it would be stripped during the fixation and preparation for dGH. Thus, dGH probe(s) used after sorting can be different than the dGH probes used in the preliminary probe hybridization. For example, the dGH probe(s) used after sorting can include two or probes that bind to different DNA target sequences on the same chromosome/chromatid and are labeled with different colored labels.

### Partitions and Arrays

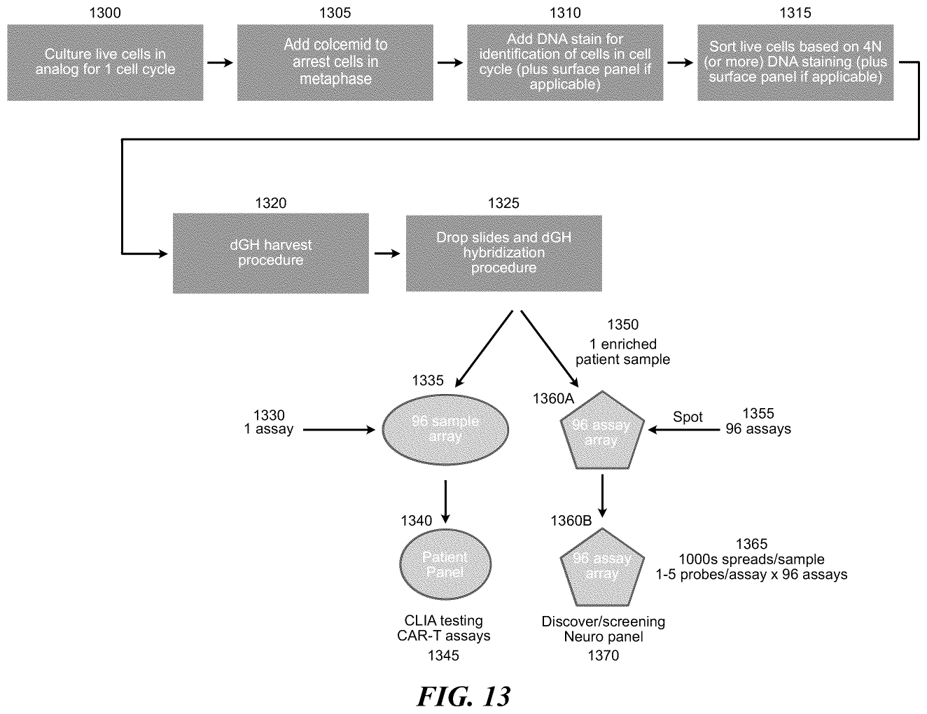

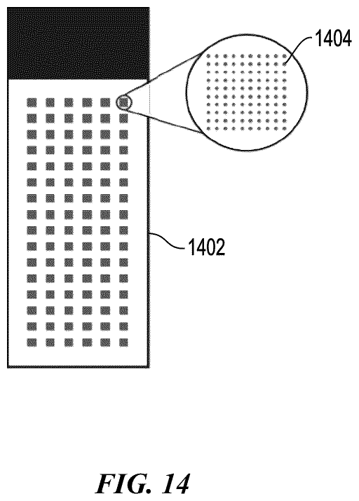

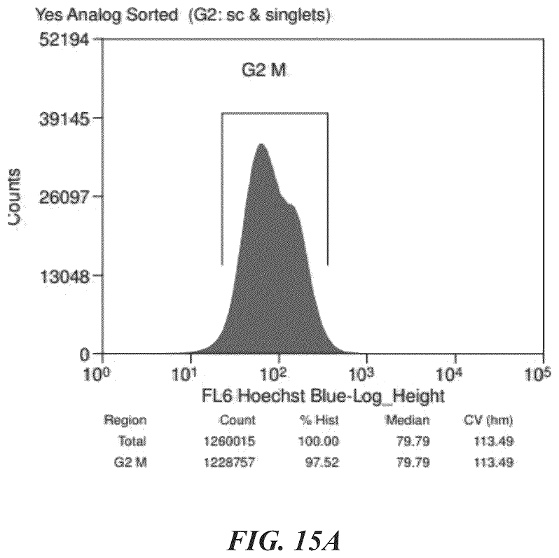

In certain aspects and embodiments herein, cells and/or chromosomes from sorted cell populations, such as metaphase-enriched cell populations, are then placed in a two-dimensional, regularly spaced arrangement on a support matrix such as by spotting onto arrays (See e.g., FIG. 13 and FIG. 14). Such arrays are typically on an object (e.g. support matrix) that is adapted for and/or compatible with, microscopic analysis. Such support matrix, or a group of 2-10 or more such support matrices (e.g. slides), having two-dimensional, regularly spaced arrangement of spots, cells, and/or chromosomes therein, themselves are aspects of this disclosure. Such aspects can include any of the details of embodiments provided herein typically in the context of methods that utilize such support matrices. For example, such array can be on a microscope slide. The array can have 2 or more (e.g. 2 to 1,000, 10,000, 100,000, or 1,000,000) addressable positions, such as 4, 8, 12, 24, 48 or 96 addressable positions, or a multiple thereof. In certain aspects, spotting is based on study of a panel of samples from one or more subjects or samples (e.g. a Patient Panel). In certain aspects, spotting is based on study of a loci or target panel of dGH probes (e.g. a Gene Panel).

Methods herein, in some embodiments include placing cells or chromosomes into one or more partitions of a two-dimensional spatial arrangement of partitions. In some non-limiting embodiments, methods herein include placing one or more cells from a population of cells in a two-dimensional spatial arrangement of partitions. In some embodiments, the cells which are placed in a two-dimensional spatial arrangement of partitions are derived from a same cell sample. In other embodiments, the cells are derived from more than one, two, three, four, or five cell sample. In some embodiments, one or more, in illustrative embodiments, two or more dGH probes are the same for each partition in which cells are placed. In some embodiments, one or more, in illustrative embodiments, two or more dGH probes in a first partition comprise at least one difference in nucleic acid sequences in comparison to the two or more oligonucleotide probes, in illustrative embodiments, dGH probes in a second partition.

FIG. 13 illustrates exemplary workflows utilizing cell sorting, directional genomic hybridization, and an array, according to certain illustrative methods disclosed herein. Cells of a population are cultured in the presence of a DNA analog which, in illustrative embodiments, can be BrdU/C (1300). Cells are then arrested in the metaphase stage to obtain a metaphase-enriched cell population which, in illustrative embodiments, can be done by adding N-methyl-N-deacetyl-colchicine (Colcemid™) to the cell culture (1305).

The cells are then stained to identify cells in a cell cycle (1310). Live cells from the population of cells cultured are sorted based on DNA staining, in illustrative embodiments, based on 4N or more DNA staining (1315). In some illustrative embodiments, DNA staining is done by Hoechst stain. In some embodiments of methods herein, the cells are further identified and/or sorted by a cell surface panel based on the surface markers that are expressed in a particular population of cells. In certain embodiments, a cell surface panel can include CD3, CD4, CD8, CD4RA, CD45RA.

The live cells obtained from the sorting step are then harvested (dGH harvest procedure) (1320), wherein, in some embodiments, the live cells are subjected to incubation, in some non-limiting embodiments, for a period of 1-20, 2-20, 5-20, in illustrative embodiments, 5-10 minutes in the presence of a hypotonic solution, which in illustrative embodiments can be a hypotonic KCl solution. In some non-limiting embodiments, the cells are further subjected to a fixation step, in illustrative embodiments in the presence of a fixative comprising methanol and acetic acid, in some non-limiting embodiments, to obtain processed cells. The processed cells are deposited or placed (or dropped) onto slides (Drop slides) (1325) to prepare metaphase spreads, in illustrative embodiments, the slides have positionally addressable arrays to produce a sorted population of metaphase spreads. In some non-limiting embodiments, the processed cells are lysed or burst open during the process of depositing or dropping onto the slides to form metaphase spreads. The metaphase spreads are contacted with one or more dGH probes, in illustrative embodiments, two or more dGH probes that are capable of hybridizing to one or more target DNA, in illustrative embodiments, two or more target DNA found on one of the single-stranded sister chromatids generated from the metaphase spreads (dGH hybridization procedure) (1325). In some embodiments of methods herein, instead of sorting cells, methods can include direct sorting of chromosomes onto arrays, wherein, in some embodiments, the chromosomes can be derived from an unsorted cell population.

In some non-limiting embodiments of an exemplary workflow illustrated in FIG. 13, the cells are T cells, and live T cells are stimulated to proliferate in vitro for a specified period of time (dependent on sample) followed by incubation with a BrdU/C analog for one cell cycle. In some non-limiting embodiments, T cells are stained for flow sorting using a memory vs effector T cell marker panel and Hoechst (to stain for cells in metaphase). In further non-limiting embodiments, T Cells are sorted using fluorescence activated cell sorting (FACS). In some non-limiting embodiments, sorted T cells undergo a dGH harvest procedure as disclosed herein to obtain processed cells. In some non-limiting embodiments, the processed cells are spotted, dropped, deposited, or placed onto slides for dGH analysis (in some non-limiting embodiments, many samples are spotted per slide and 1-3 assays are performed per slide). In illustrative non-limiting embodiments, methods herein include analyzing CAR-T cells (1330, 1335, 1340, and 1345). In non-limiting embodiments, CAR-T cells are analyzed by a CLIA-certified assay (CLIA-testing CAR-T assays) (1345), wherein multiple samples or patients are enriched for sub-populations in metaphase, which, in non-limiting embodiments, are deposited or dropped onto a slide or an array, for example, 96 sample array (1335) then hybridized with a single dGH probe, in non-limiting embodiments, a panel of probes (Patient Panel) (1340) that is made up of a pool of oligonucleotides that bind their complementary DNA sequence within a target DNA sequence, or a set of dGH probes (i.e. one assay) (1330).

In some non-limiting embodiments of an exemplary workflow illustrated in FIG. 13, the cells are iPSCs and live iPSC are incubated with DNA analog (e.g. BrdU) for one cell cycle. Cell sample is stained for flow sorting using BrdU/Hoechst method (to stain for cells in metaphase). Cells are sorted using fluorescence activated cell sorting (FACS). Sorted cells, in some non-limiting embodiments, are subjected to a dGH harvest procedure as disclosed herein, and spotted (dropped, deposited, or placed) onto slides, in illustrative embodiments, onto arrays with addressable positions for dGH analysis, in illustrative embodiments, one sample (1350) is used for 96 assays (1355) on a single slide, in non-limiting embodiments, on a single 96-assay array (1360A). In some non-limiting embodiments, the exemplary workflow includes at least 1000 spreads per sample on the array, and each assay in the array includes hybridization with 1-5 oligonucleotide probes, in illustrative embodiments, dGH probes, and wherein the array is a 96-assay array (1365, and 1360B). In some non-limiting embodiments, the exemplary workflow includes a discovery/screening panel (1370), where a single sample is enriched for metaphase cells, spotted onto a slide (1350), then probed with 96 different panels of probes (i.e., assays) (1355). For these types of screening methods, there would be a 96-probe assay designed against known disease targets that can be screened for in each spot on the slide. In some non-limiting embodiments, the probes, in illustrative embodiments, targeted dGH probes would be designed against genes that are known to be involved in a neurodegenerative disease. Some non-limiting examples of neurodegenerative diseases include amyotrophic lateral sclerosis, multiple sclerosis, Parkinson's disease, Alzheimer's disease, Huntington's disease, multiple system atrophy, and prion diseases.

The exemplary workflow illustrating enriched array strategies depicted in FIG. 13 are not limited to CAR-T cells or neurodegenerative targets specifically and are used as example applications of these strategies only. Note that CAR-T cells are T cells which are edited such that they express a chimeric antigen receptor (CAR) that is specific for a predetermined antigen. In some embodiments, CRISPR editing can be used to remove expression of endogenous receptors. Thus, some embodiments herein analyze CAR-T cells that include endogenous receptors, and some embodiments analyze CAR-T cells that have been edited to remove endogenous receptors.

In certain aspects, methods herein can include placing or depositing chromosomes into one or more partition of a two-dimensional spatial arrangement of partitions to form partitioned chromosomes. In some embodiments, a two-dimensional spatial arrangement can include a positionally addressable array. Some embodiments can include placing the chromosomes from un-sorted population of cells into one or more partition of a two-dimensional spatial arrangement of partitions to form partitioned chromosomes. Some other embodiments can include placing the chromosomes from a sorted population of cells into one or more partition of a two-dimensional spatial arrangement of partitions to form partitioned chromosomes. In some embodiments, partitioned chromosomes can be derived from the same cell sample. In other embodiments, partitioned chromosomes can be derived from more than one, two, three, four, or five cell samples. In some embodiments of methods that include partitioned chromosomes, one or both of a pair of single-stranded sister chromatids generated from each of the chromosomes are contacted with one, two, three, four, five, or more dGH probes. In some embodiments, one or both of a pair of single-stranded sister chromatids generated from each of the chromosomes are contacted with two or more dGH probes, wherein the two or more dGH probes are the same for each partition. In other embodiments, the two or more dGH probes are different for each partition. In some embodiments, the two or more dGH probes in a first partition comprise at least one difference in nucleic acid sequences in comparison to the two or more dGH probes in a second partition. In some embodiments, one or more partitions of a two-dimensional spatial arrangement of partitions can comprise isolated nucleic acid, such as, but not limiting to DNA, cDNA, RNA, and mRNA. In some embodiments, the isolated DNA can be a stretched DNA.

In certain aspects of methods as described herein, the two-dimensional spatial arrangement is a grid. In some embodiments, the grid provides for a predetermined hybridization pattern using one, two, three, four, five or more consistent hybridization probes, in illustrative embodiments, dGH probes across specified X and Y coordinates.

FIG. 14 illustrates a substrate which can be utilized for a two-dimensional spatial arrangement or array as disclosed herein. In illustrative embodiments, the substrate as illustrated in FIG. 14 can be utilized to place, drop or deposit cells, in illustrative embodiments, sorted cells, or to place or deposit chromosomes/chromatids, in some illustrative embodiments, sorted chromosomes/chromatids in a two-dimensional spatial arrangements, which in some embodiments include addressable positions. In some non-limiting embodiments, the substrate as illustrated in FIG. 14 can be utilized to place, drop, or deposit cells that are lysed, for example, cell lysate that comprises chromosomes. In some non-limiting embodiments, one exemplary protocol for two-dimensional arrangement of cells and/or chromosomes in partitions on arrays include a layout of cells or chromosomes to be arrayed which in some non-limiting embodiments include specifying: number and spacing of wells, and number of cells per well, and for multiple samples, identifying which samples are to be spotted in each well of the arrays. In some embodiments, cells are fixed with a fixative as used for dGH or FISH, which in illustrative embodiments, can be an organic solvent fixative, such as a methanol and/or acetic acid fixative, such as a 1:1, 2:1, 3:1, 4:1 or 5:1 methanol:acetic acid fixative solution. In some non-limiting embodiments, the cell sample and/or chromosome sample obtained after the fixing step are dispensed, deposited or placed onto a microscopic slide using an instrument, such as, but not limited to, cellenONE® X1. In some non-limiting embodiments, after the cell samples and/or chromosome samples are deposited onto a microscopic slide, the slides are allowed to dry to immobilize the cells and/or chromosomes in a two dimensional spatial arrangement of partitions on a solid support (FIG. 14, 1402). In some embodiments, the cells and/or chromosomes from each sample of a plurality of samples can be deposited onto the same addressable position or location within each partition (FIG. 14, 1404).

### Banded dGH Embodiments

In some aspect or embodiments, methods herein include methods for generating a multi-color fluorescence pattern on a single-stranded sister chromatid of a pair of single-stranded sister chromatids, comprising the following steps, which in some embodiments are further included in other methods herein: (a) generating the pair of single-stranded sister chromatids from a chromosome; (b) contacting one or both single-stranded sister chromatids with two or more directional genomic hybridization (dGH) probes each comprising a fluorescent label from a set of at least two fluorescent labels capable of emitting different colors; (c) performing fluorescence analysis of one or both single-stranded sister chromatids of the pair by detecting fluorescence signals generated based on a hybridization pattern of the two or more dGH probes to the single-stranded sister chromatid; and (d) generating, based on the fluorescence analysis, the multi-color fluorescence pattern on the single-stranded sister chromatid. In illustrative embodiments, the multi-color fluorescence pattern comprises bands having the different colors of the at least two fluorescent labels. Such methods are examples of banded dGH methods. The multi-color fluorescent pattern can be used, for example, to detect and/or classify at least one structural feature, such as a structural variant or to detect a chromosome repair event.

In some aspect or embodiments, methods herein include methods for detecting and/or classifying at least one structural feature and/or repair event of a chromosome of a cell, comprising the following steps, which in some embodiments are further included in other methods herein: (a) generating a pair of single-stranded sister chromatids from the chromosome, wherein at least one of the sister chromatids comprises two or more target DNA sequences; (b) contacting one or both single-stranded sister chromatids with two or more uni-directional genomic hybridization (dGH) probes in a metaphase spread generated from the cell, wherein each dGH probe comprises a pool of single-stranded oligonucleotides complementary to at least a portion of one of the two or more target DNA sequences and comprising the same label, and wherein at least two, three, four or five of the two or more dGH probes each bind to a different one of the two or more target DNA sequences and each comprise a label of a different color; (c) performing fluorescence analysis of one or both single-stranded sister chromatids by detecting fluorescence signals generated based on a hybridization pattern of the at least two, three, four, or five dGH probes to one or both single-stranded sister chromatids of the pair; and (d) detecting, based on the fluorescence analysis, the presence of the structural feature and/or the chromosome repair event. In some embodiments, pools of the single-stranded oligonucleotides complementary to said two or more target DNA sequence on at least one of said single-stranded sister chromatid comprise labels of at least three, four, five, six, seven, eight, nine, or ten different colors. In some embodiments, the dGH probe comprising a pool of single-stranded oligonucleotides complementary to said target DNA sequence on each single-stranded sister chromatid comprise labels of between 2 and 30, 2 and 25, 2 and 20, 2 and 15, 3 and 30, 3 and 20, 3 and 10, 5 and 30, 5 and 25, 5 and 20 different colors, in illustrative embodiments, between 2 and 10 different colors. The methods can be used to detect, for example, a chromosome structural variant and/or a sister chromatid exchange repair event. In some embodiments, the method further comprises comparing the fluorescence analysis with reference fluorescence information representing a control sequence. Fluorescence analysis can include generating spectral measurements or generating a fluorescence pattern, from one or both single-stranded sister chromatids. The fluorescence pattern in illustrative embodiments is a multi-color banding pattern, and the method can be referred to herein as banded dGH or multi-color banded dGH.

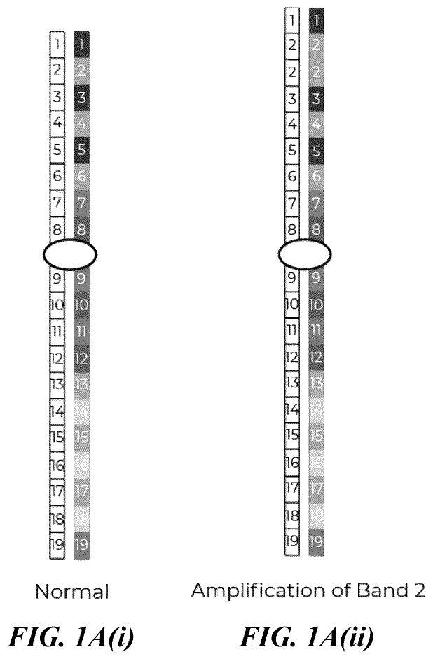

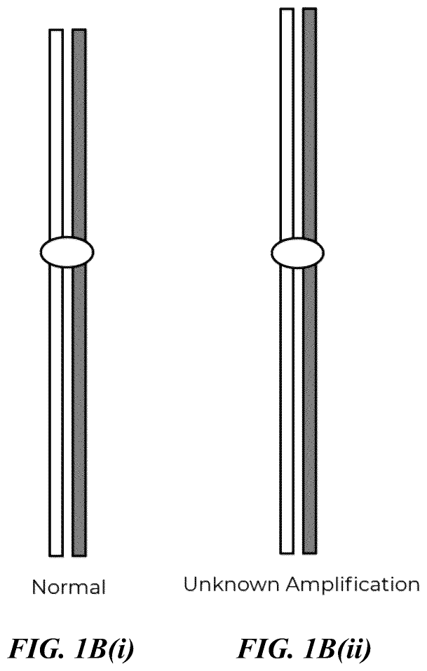

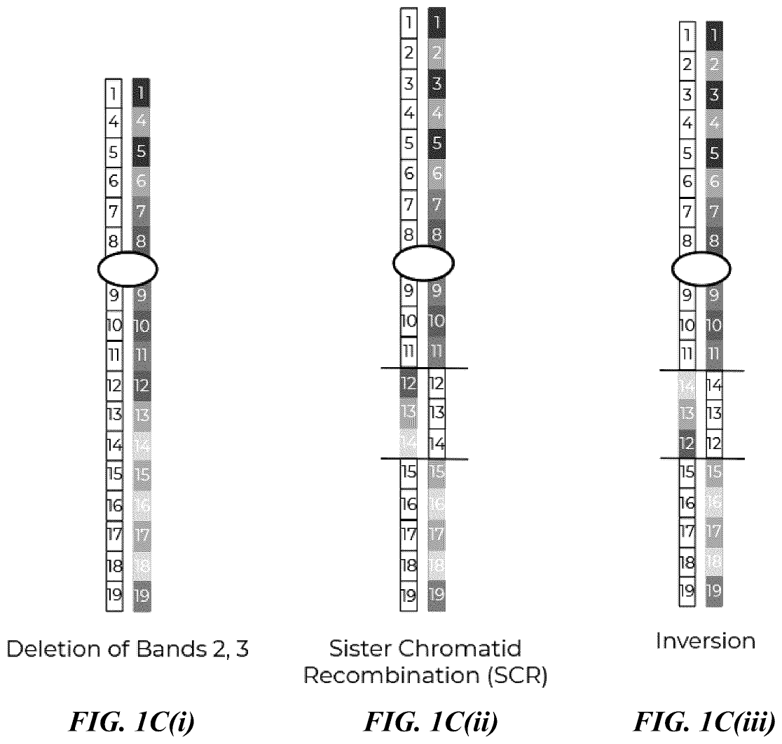

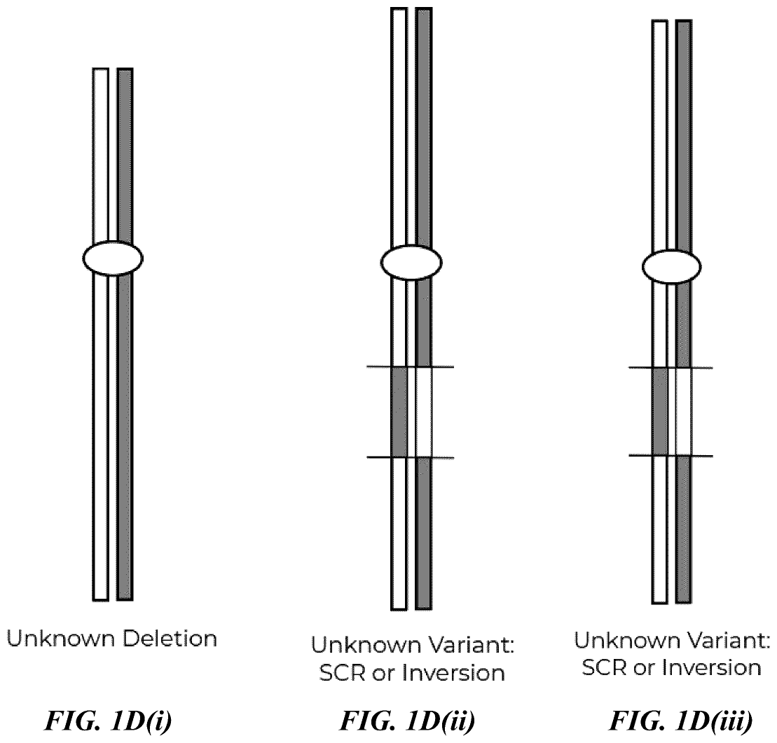

FIG. 1A-FIG. 1D provide diagrams to illustrate an example of intra-chromosomal rearrangements that can be detected by banded dGH analysis versus a monochrome dGH paint that uses only a single color dGH probe as opposed to two or more dGH probes of different colors, which are used in the banded dGH. It can be appreciated that the amplification of band 2 can be observed in FIG. 1A(ii) that uses banded dGH versus FIG. 1B(ii) that uses monochrome dGH. Similarly, the events, deletion, sister chromatid recombination (SCR), and inversion are identifiable in FIG. 1C(i), FIG. 1C(ii), and FIG. 1C(iii), respectively, which illustrates the banding pattern of banded dGH as compared to FIG. 1(D)(i), FIG. 1(D)(ii), and FIG. 1D(iii) which illustrates monochrome dGH.

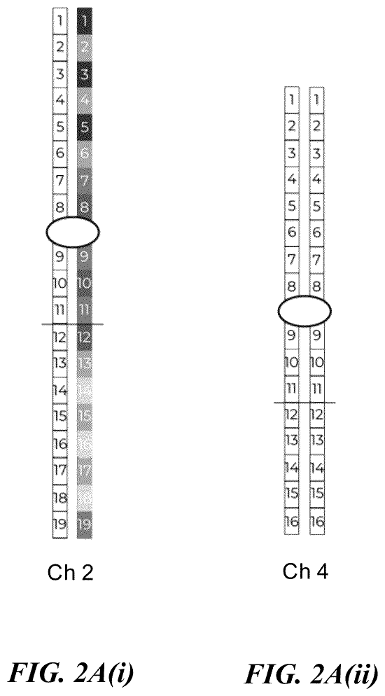

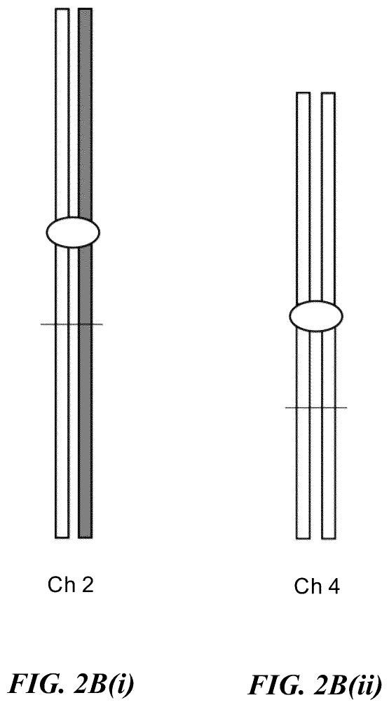

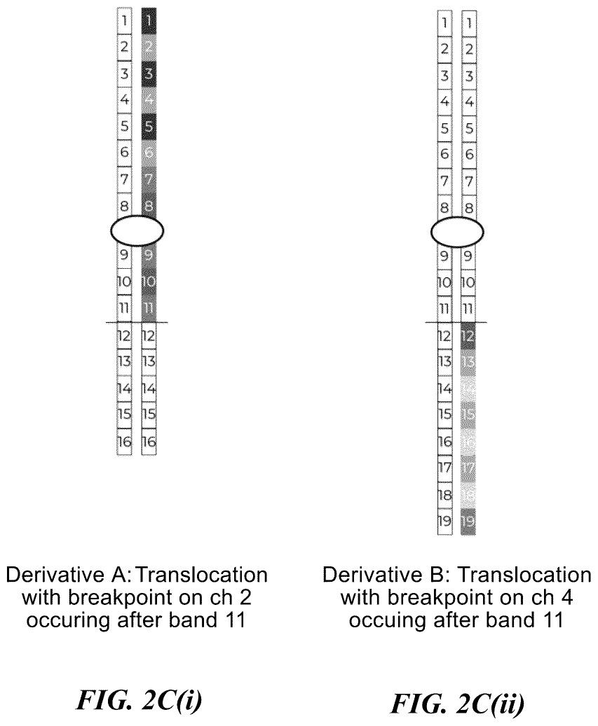

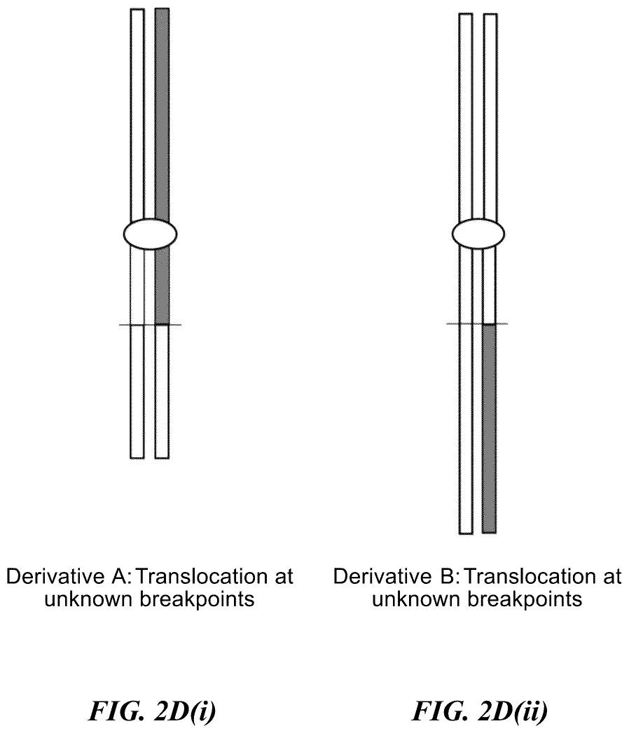

FIG. 2A-FIG. 2D provide diagrams to illustrate an example of the colors of chromosomes after inter-chromosomal rearrangements (translocations between two different chromosomes), using banded dGH (FIG. 2A(i), FIG. 2A(ii), FIG. 2C(i), FIG. 2C(ii)) vs monochrome dGH paint methods (FIG. 2B(i), FIG. 2B(ii), FIG. 2D(i), FIG. 2D(ii)). It can be appreciated from FIG. 2C(i) that the product of reciprocal translocation, with material from Ch 2 (bands 1-11) fused with material from Ch 4 (unpainted) is identifiable. Further, from FIG. 2C(ii) another product of reciprocal translocation, with material from Ch 2 (bands 12-19) fused with material from Ch 4 (unpainted) is identifiable. Whereas no breakpoint location of translocation can be identifiable using monochrome paints (FIG. 2D(i) and FIG. 2D(ii)).

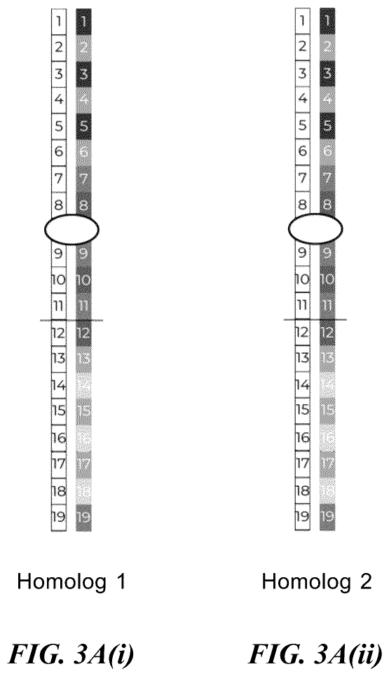

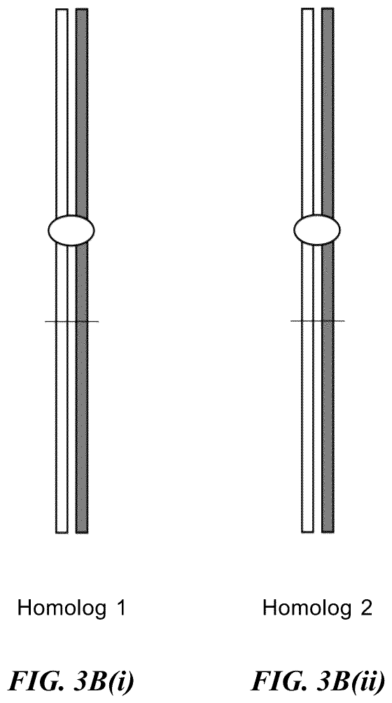

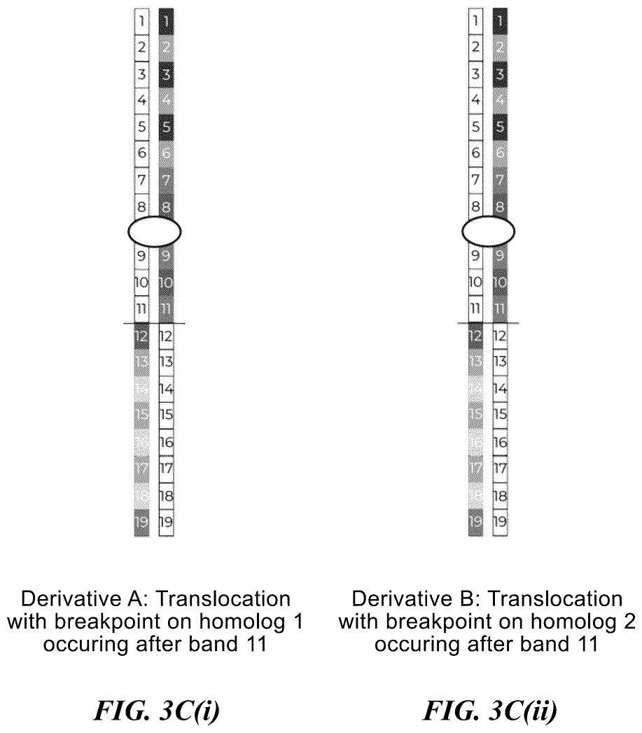

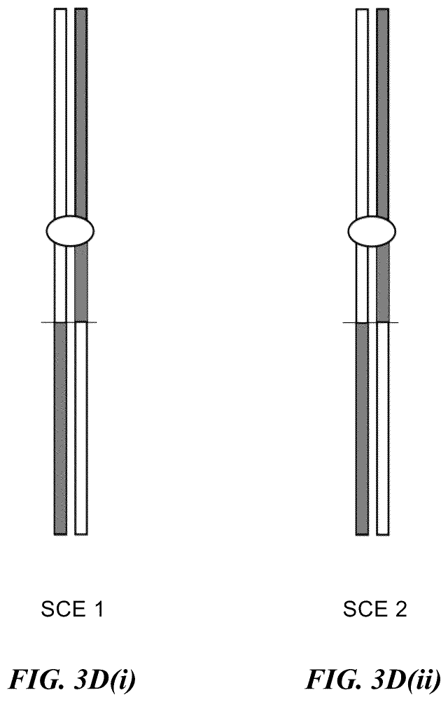

FIG. 3A-FIG. 3D illustrate an example of inter-chromosomal allelic rearrangements (translocations between two homologs of the same chromosome) and their detection by banded dGH (FIG. 3A(i), FIG. 3A(ii), FIG. 3C(i), and FIG. 3C(ii)) vs monochrome dGH (FIG. 3B(i), FIG. 3B(ii), FIG. 3D(i), and FIG. 3D(ii)). From FIG. 3C(i) the product of reciprocal translocation between homologs, with material from Ch 2 homolog 1 exchanged with material from Ch2 homolog 2 at the same breakpoint (between bands 11 and 12) is identifiable. Similarly, from FIG. 3C(ii) the product of reciprocal translocation between homologs, with material from Ch 2 homolog 1 exchanged with material from Ch2 homolog 2 at the same breakpoint (between bands 11 and 12) is identifiable. Whereas from FIG. 3D(i) it can be appreciated that the product of reciprocal translocation between homologs, with material from Ch 2 homolog 1 exchanged with material from Ch2 homolog 2 is at unknown breakpoints. Also, from FIG. 3D(ii) the product of reciprocal translocation between homologs, with material from Ch 2 homolog 1 exchanged with material from Ch2 homolog 2 is at unknown breakpoints. Statistical chances of two SCEs at the exact same location on each homolog is very unlikely, versus an allelic translocation event being quite likely especially in a cell being edited at a single location (two DSBs—one per homolog) but cannot be confirmed with monochrome paint due to lack of genomic coordinate specificity.

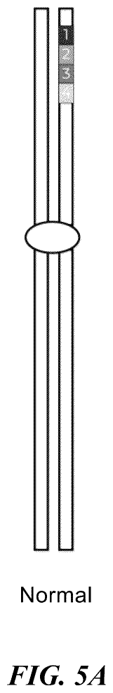

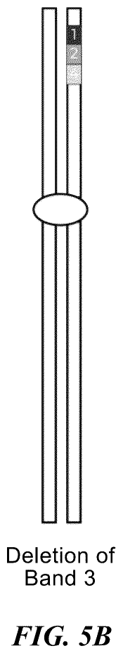

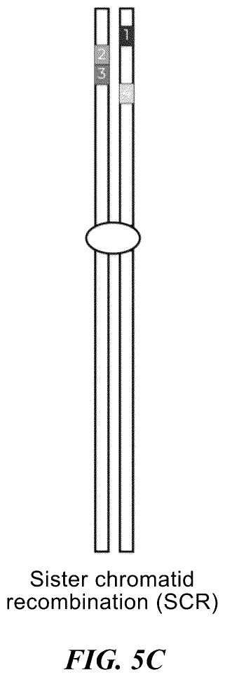

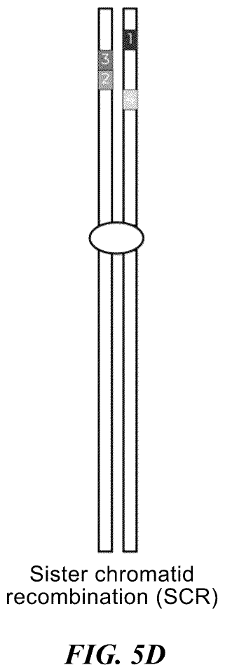

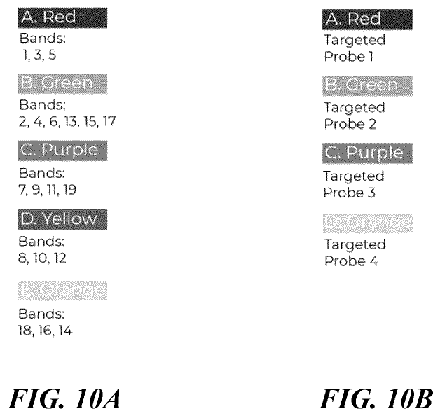

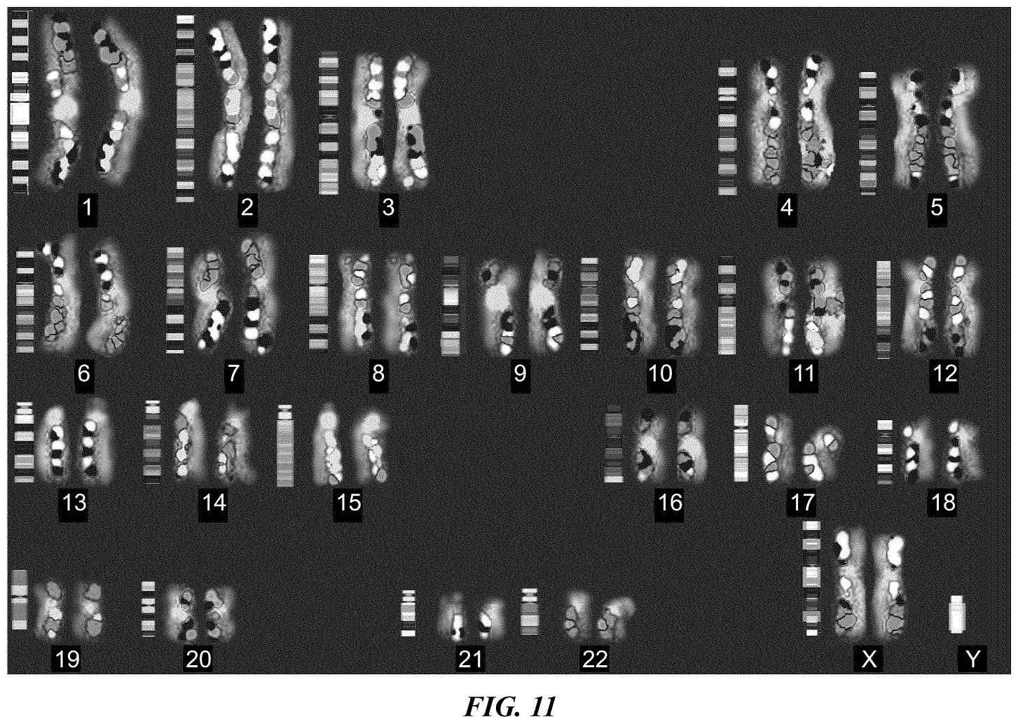

FIG. 5A-FIG. 5D illustrate an example of using Targeted Probe dGH Assays for SV detection. In this method, dGH probes can be designed to target loci within a genome of interest, for example, loci known to influence or cause a disease state with known locations, telomeric locations, or subtelomeric locations. Using this method, structural variations such as, but not limited to, deletions of a portion of a locus of interest, or inversions within in a normal repair event can be identified as shown for chromosome 2 in FIG. 5B and FIG. 5D, respectively, when compared to targeted banding pattern of a normal, reference chromosome (shown in FIG. 5A for comparison to FIG. 5B, and FIG. 5C for the normal banding pattern seen in sister chromatid recombination (SCR) in relation to FIG. 5D). Further details described below and the color map for the grayscale images is shown in FIG. 10B. FIG. 5A shows normal Chromosome 2, prepared for dGH, hybridized with 4 targeted probes around a locus of interest. FIG. 5B shows chromosome 2 with deletion of portion of the locus of interest (spanning the genomic coordinates covered by targeted probe 2). FIG. 5C shows chromosome 2 with a sister chromatid recombination event, with targeted probes 2 and 3 seen on the opposite sister chromatid from targeted probes 1 and 4, with the order of the probes maintained—1, 2, 3, 4 from telomere to centromere. FIG. 5D shows chromosome 2 with an inversion event, where targeted probes 2 and 3 can be seen on the opposite sister chromatid from targeted probes 1 and 4, with the order of probes 2 and 3 reversed. Probes appear in 1, 3, 2, 4 order from telomere to centromere.

Accordingly, methods herein can include a set of banded dGH probes in the contacting step to generate a set of banded dGH fluorescence patterns. Methods are disclosed for the detection of structural variations or repair events in chromosomes by labeling of single-stranded chromatids with dGH probes of different colors. A dGH probe is typically a pool of individual single-stranded oligonucleotides that are labeled with the same fluorescent label of a set of fluorescent labels, wherein each single stranded oligonucleotide of a pool binds a different complementary DNA sequence within the same target DNA sequence. The hybridization pattern of the pool of labeled, single-stranded oligonucleotides produce a fluorescence pattern, such as a spectral profile, which enables high-resolution detection of structural variations and repair events, facilitating distinction of benign variations from deleterious structural variations. Further, the spectral profile provides information regarding complex structural variations where more than one rearrangement of chromosomal segments may have occurred.

Accordingly, in one aspect, provided herein is a method for detecting at least one structural variation and/or repair event in a chromosome from a cell, the method comprising the steps of:
(a) performing a directional genomic hybridization (dGH) reaction by contacting a pair of single-stranded sister chromatids generated from the chromosome in a metaphase spread prepared from the cell, with two or more dGH probes, each dGH probe comprising a fluorescent label of a set of fluorescent labels, wherein each dGH probe comprises a pool of single-stranded oligonucleotides that comprise a same fluorescent label of the set of fluorescent labels, wherein each single stranded oligonucleotide of a pool binds a different complementary DNA sequence within a same target DNA sequence found on one of the single-stranded sister chromatids, wherein at least two of the two or more dGH probes each binds to a different target DNA sequence on one of the single-stranded sister chromatids and each comprises a fluorescent label of a different color;
(b) generating a fluorescence pattern from one or both single-stranded sister chromatids using fluorescence detection, wherein the fluorescence pattern is based on a hybridization pattern of the two or more dGH probes to one or both single-stranded sister chromatids of the pair; and
(c) detecting based on the fluorescence pattern, the presence of the at least one structural feature, which in non-limiting embodiments is a structural variation and/or repair event in the chromosome from the cell.

Accordingly, in one aspect, provided herein is a method for detecting at least structural variation and/or repair event in a chromosome from a cell, the method comprising the steps of:
(a) generating denatured chromosomes comprising single stranded chromosomes from each of the chromosomes present in one or more partitions, wherein at least one of the single-stranded chromosomes comprises a target DNA sequence of the target chromosome; and
b) contacting the single stranded chromosomes with the first dGH probe, wherein the first dGH probe comprises a pool of single-stranded oligonucleotides complementary to a portion of a first target DNA sequence on one or more of the single-stranded denatured chromosomes and comprising a first colored fluorescent label; and
(c) performing fluorescence analysis of the chromosomes comprising the target chromosome present in one or more partitions, by detecting fluorescence signals generated based on a hybridization pattern of the first dGH probe to the single-stranded chromosomes comprising the target chromosome.

In some embodiments, the detecting based the fluorescence pattern comprises
(c) (i) comparing the fluorescence pattern of the one or both single-stranded sister chromatids to a reference fluorescence pattern representing a control sequence; and
(c) (ii) detecting at least one difference between the reference fluorescence pattern and the fluorescence pattern of the one or both single-stranded sister chromatids of the pair. Typically, single stranded chromatids are generated by a process in which a DNA analog (e.g. BrdU) is provided to an actively dividing cell for a single replication cycle, which is then incorporated selectively into the newly synthesized daughter strand, a metaphase spread is prepared, the incorporated analog is targeted photolytically to achieve DNA nicks which are used to selectively enzymatically digest and degrade the newly synthesized strand, resulting in a single-stranded product. If we use the terms Watson and Crick to describe the 5′ to 3′ strand and 3′ to 5′ strand of a double-stranded DNA complex, an untreated metaphase chromosome will have one sister chromatid with a parental Watson/daughter Crick, one sister chromatid with a daughter Watson/parental Crick. In the chromosomes prepared according to the method above, one sister chromatid will consist of the Parental Watson strand only, and the other sister chromatid will consist of the parental Crick strand only.
   Directional Genomic Hybridization (dGH)

Methods herein typically include detecting the fluorescent labels on dGH probes used in FISH reactions, or in illustrative embodiments in dGH reactions, in metaphase spreads using fluorescent analysis, such as using fluorescence microscopy. A standard dGH protocol typically includes steps 1-4 of the following workflow, and certain illustrative embodiments include 1 or more of steps 6-10 as well:
1) Cell culture with analog addition
2) Harvest and Fixing
3) Metaphase Dropping
4) Stripping
5) Hybridization of dGH Probes
6) Overview Image
7) Metaphase Imaging
8) Image Qualification
9) “Scoring” or individual image (single cell) analysis
10) Population analysis Single-stranded chromatids may be generated by any means known in the art, including but not limited to the CO-FISH technique.

FIG. 12 illustrates an exemplary workflow for an assay utilizing directional genomic hybridization according to certain illustrative methods disclosed herein. Cells of a population are cultured for at least 1 cell cycle in the presence of a DNA analog which, in illustrative embodiments, can be BrdU/C (1200) to prepare hemi-substituted chromosomes/hemi-substituted sister chromatids. In some non-limiting embodiments, cells are then arrested in the metaphase stage to obtain a metaphase-enriched cell population which, in illustrative embodiments, can be done by adding N-methyl-N-deacetyl-colchicine (Colcemid™) to the cell culture (1205). The cells are then harvested (dGH harvest procedure) (1210), wherein, in some non-limiting embodiments, the cells can be harvested by a dGH harvest procedure, which in illustrative embodiments include incubating the cells, in some non-limiting embodiments, for a period of 1-20, 2-20, 5-20, in illustrative embodiments, 5-10 minutes in the presence of a hypotonic solution, which in illustrative embodiments can be a hypotonic KCl solution. In some non-limiting embodiments, the cells are further subjected to fixation by exposing the cells to a fixative, which in some non-limiting embodiments, can comprise methanol and acetic acid, in a volume ratio of 1:1 to 5:1, in illustrative embodiments, 3:1 to form processed cells. In some embodiments, the processed cells are deposited (dropped, or placed) onto slides (Drop slides) (1215) to form metaphase spreads. The metaphase spreads are contacted with one or more dGH probes, in illustrative embodiments, two or more dGH probes that are capable of hybridizing to one or more target DNA, in illustrative embodiments, two or more target DNA found on one of the single-stranded sister chromatids generated from the metaphase spreads (dGH hybridization procedure) (1215). In some non-limiting embodiments, a single un-sorted sample enriched for metaphase cells, spotted onto a slide is probed with oligonucleotide probes, in illustrative embodiments, dGH probes in 1-2 different assays (1230). In some non-limiting embodiments, the exemplary workflow includes 10 to 500 spreads per sample, and 1-5 oligonucleotide probes (1225), in illustrative embodiments, dGH probes per assay. In some embodiments, at least one non-enriched sample (1220) can be included in the exemplary workflow as illustrated in FIG. 12.

Oligonucleotides of a pool of two or more single-stranded oligonucleotides that make up a dGH probe are capable of hybridizing to single-stranded chromatids and can be of any functional length. Without limitation to any particular embodiment, the single-stranded oligonucleotides can be, for example, 10 to 100 nucleotides in length, 15 to 90 nucleotides in length, 25 to 75 nucleotides in length, 30 to 50 nucleotides in length, or 37 to 43 nucleotides in length, or any combination thereof. In some embodiments, single-stranded oligonucleotides can be of at least 10, 20, 50, 70, 100, 150 or more nucleotides in length.

In certain embodiments, dGH probes for the methods disclosed herein can range in number of oligonucleotides in the pool of oligonucleotides that make up the dGH probe, from a small number of oligonucleotides directed to specific chromosomal regions, on one or more than one chromosome, providing locus specific banding on a limited number of chromosomal regions (e.g. one or more chromosomal regions), to one or more than one gene of interest or a larger number of oligonucleotides that target all known genes on a single-stranded chromatid, on several single-stranded chromatids, on a group of single-stranded chromatids, on all single-stranded sister chromatids, on a single chromosome, on a group of chromosomes, or on all the chromosomes in the organism under study. In some embodiments, a dGH probe can include for example, between 10 and 2×10⁶, 1,000 and 2×10⁶, 10,000-100,000, 10,000-50,000, 10-10,000, 100-5,000, 100-1,000, 100-500, 200-1,000, 200-500 single-stranded oligonucleotides, each with a different nucleic acid sequence.

Probes capable of hybridizing to single-stranded chromatids, in illustrative embodiments dGH probes, can be of any functional length. Without limitation to any particular embodiment, probes can be 10 to 100 nucleotides in length, 15 to 90 nucleotides in length, 25 to 75 nucleotides in length, 30 to 50 nucleotides in length, 37 to 43 nucleotides in length or any combination of low end and high end thereof.

In certain aspects, sets of labeled probes for the methods disclosed herein can range in number of probes from smaller probe sets directed to specific chromosomal regions, on one or more than one chromosome, providing locus specific banding on a limited number of chromosomal regions (e.g., one or more chromosomal regions), or larger probe sets providing arrays of probes targeting chromosomal regions throughout the genome. In some embodiments, the number of probes in a particular set of probes can vary, starting from at least 1 probe in a set to more than 1, 10, 20, 30, 50, 75, or 100 probes in a set. In some embodiments, there is at least 1 probe, or 2 probes in a set. In some embodiments, there can be 1-100, 1-90, 1-80, 1-70, 1-60, 1-50, 1-40, 1-30, 1-20, or 1-10 probes in a set. In some embodiments, there can be more than 100, 200, 300, 400, or 500 probes in a set. In some illustrative embodiments, a set of labelled probes includes a set of dGH probe(s).

In some embodiments, a probe can include for example, between 10 and 2×10⁶, 1,000 and 2×10⁶, 10-10,000, 100-5,000, 100-1,000, 100-500, 200-1,000, 200-500 single-stranded oligonucleotides, each with a different nucleic acid sequence. In some embodiments of methods as disclosed herein, a set of labeled probes can be dGH probe. In some embodiments, a dGH probe can comprise at least 10, 20, 50, 75, 100, 200, 500, or 1,000 single-stranded oligonucleotides. In some embodiments, a dGH probe can comprise between 1,000 to 100,000 single stranded oligonucleotides, each with a different nucleic acid sequence. In some embodiments, the complementary sequences of the dGH probes may be relatively equally dispersed throughout a genome. In other embodiments, the complementary sequences of the dGH probes can be more concentrated in certain regions of a genome and more dispersed in other regions of a genome. In certain embodiments, the pool of labeled single-stranded oligonucleotides in each dGH probe for the methods disclosed herein can range in number of oligonucleotides from a small number of oligonucleotides directed to specific target DNA sequences such as specific chromosomal regions on one chromosome, providing for example, locus specific banding on a limited number of chromosomal regions (e.g., one or more chromosomal regions), to a dGH probe having a larger number of single-stranded oligonucleotides, for example that in some embodiments can detect larger target DNA sequences, such as larger chromosomal regions. In certain embodiments, each dGH probe of a set of uni-directional dGH probes binds a target DNA sequence that is on the same single-stranded sister chromatid and comprises a different fluorescent label excited by, and/or emitting a different color such that through fluorescence analysis after a dGH reaction, a multi-color banding pattern is obtained on a single-stranded sister chromatid. Such a set can be referred to as a set of multi-colored dGH probes.

dGH reactions typically involve the generation of single-stranded chromatids. Such single-stranded chromatids can be generated by any means known in the art. In illustrative embodiments, single-stranded chromatids are generated using the CO-FISH technique. As described in “Strand-Specific Fluorescence in situ Hybridization: The CO-FISH Family” by S. M. Bailey et al., Cytogenet. Genome Res. 107: 11-14 (2004), chromosome organization can be studied using strand-specific FISH (fluorescent or fluorescence in situ hybridization), which is often referred to as CO-FISH or Chromosome Orientation-FISH. The CO-FISH technique requires cultivation of cells in the presence of bromodeoxyuridine (BrdU) and/or bromodeoxycytidine (BrdC) for a single round of replication (a single S phase). Cells can be incubated in nucleotide analog for a period of time, for example for between 12 and 52 hours, where the time is based on the length of a culture's cell cycle. Each newly replicated double helix contains one parental DNA strand plus a newly synthesized strand in which the nucleotide analogs have partially replaced thymidine and/or deoxycytidine. Following preparation of metaphase chromosomes on microscope slides by standard cytogenetic techniques, the cells are exposed to UV light in the presence of the photosensitizing DNA dye Hoechst, which results in numerous strand breaks that occur preferentially at the sites of BrdU incorporation. Nicks produced in the chromosomal DNA by this treatment then serve as selective substrates for enzymatic digestion and degradation by Exo III. This results in the specific removal of the newly replicated strands while leaving the original (parental) strands largely intact. Thus, for the purposes of subsequent hybridization reactions, the two sister chromatids of a chromosome are rendered single stranded, and complementary to one another, without the need for thermal denaturation. The intact parental strands then serve as single stranded target DNA for hybridization with pools of single-stranded oligonucleotide probes. CO-FISH was designed to determine the orientation of tandem repeats within centromeric regions of chromosomes. Mammalian telomeric DNA consists of tandem repeats oriented in 5′->3′ towards the termini of all vertebrate organisms. In CO-FISH, single-stranded oligonuclotides were directed to tandem repeat sequences in the telomeres.

In some embodiments, extended chromatids are analyzed in a dGH method, for example to improve resolution of the dGH bands generated during such a method. Such extended chromatids can be used to improve the resolution of fluorescence signals and resulting fluorescence banding patterns, during methods herein. Extended chromosomes or chromatids can be selected during analysis of chromatids in a metaphase spread from a dGH reaction using analysis software of a fluorescence detection and/or analysis system. During image analysis, chromatids generated from the same chromosome, such as a particular human chromosome (e.g. human chromosome 2), can appear as more or less condensed (e.g. stretched vs. stubby) in a metaphase spread to a technician's visual observation. Cytogenetic analysis software can be part of a fluorescent analysis system used to carry out methods herein, and can include functionality to measure the length, width, and length to width ratio of chromosomes and/or chromatids on a metaphase spread. This information can be used for example, to select longer single-stranded chromatids on a metaphase spread that were generated from a particular chromosome.

Intercalating agents such as those used in cytogenetic analysis (e.g ethidium bromide) can be used as part of a dGH analysis method herein to obtain elongated chromosomes for fluorescence analysis. In such workflows, cells can be incubated in nucleotide analog, for example for between 12 and 52 hours depending on the length of the culture's cell cycle, before optionally a chelator is added to the culture media before further processing for the dGH analysis. Thus, in some embodiments, cultured cells are incubated with an intercalating agent before metaphase spreads are prepared on microscope slides, and thus before a pair of single-stranded chromatids are contacted with dGH probes in the metaphase spread in methods disclosed herein.

In some embodiments, chromosomes can be elongated or stretched using sheer force. In such embodiments, the chromosomes can be stretched on a slide. In some embodiments, an array of chromosomes on a slide are manually stretched, such as on a positional addressable array. This process can precede oligonucleotide probe hybridization. In some embodiments, the chromosomes are stretched according to known methods, such as DNA combing methods. In some embodiments, matrix proteins are removed to relax the chromosome before hybridization of oligonucleotide probes, such as dGH probes or oligonucleotides probes used in PinPoint FISH. In some embodiments, chromosomes on a positionally addressable array are stretched, for example such that they are 10, 20, 25, or 50% larger than chromosomes that have not been stretched or are in compact configuration on a metaphase spread or array. In some embodiments, internal control dGH probe ladders are used in methods herein to assess the limit of detection and the resolvability of two fluorescent spots in close proximity in any particular metaphase spread during analysis of the results of a particular performance of a method herein. The chromosome condensation (compact vs long) in metaphase spread preparations varies between cells and between cell preparations. This material variability can be accounted for in an assessment before determining the resolution of structural variation classification, detection and/or determination in performance of a dGH analysis. For example, in longer, more stretched configurations of chromatin, hybridization signals from dGH probes spaced close together can be resolved as separate signals, and in more compact and condensed chromatin, hybridization signals from dGH probes spaced closely together will appear as a single merged signal. Thus, internal control dGH probe ladders can be included to determine the limit of detection and/or the resolvability of two spots in close proximity.

Accordingly, in some embodiments of methods herein, a set of control dGH probes can be included that form internal control dGH probe ladders that have the properties of dGH probes disclosed herein, but bind to a control single-stranded sister chromatid. The ladder can have at least 3 (e.g. 3-50, 3-25, 3-20, 3-15, 3-10, or 3-5) control dGH probes that bind to target DNA sequences on a control single-stranded sister chromatid. The control single-stranded sister chromatid can be the other single strand chromatid of a pair of single-strand chromatids that are generated from an on-test target chromosome that is being analyzed for the presence of a structural feature such as a structural variant or repair event. Alternatively, the control single-stranded sister chromatid can be from another chromosome. The control dGH probes of a control dGH probe ladder in illustrative embodiments can have the following properties:
i) each control dGH probe of a ladder can have a different number of single-stranded oligonucleotides (such number can be for example, between 10 and 1×10⁶) and can differ between control dGH probes of the ladder by 10, 100, 1,000, 10,000 or 100,000 oligonucleotides;
ii) each control dGH probe of a ladder can have a number of single stranded oligonucleotides that is within 10, 9, 8, 7, 6, 5, 4, 3, 2, 1 of each other (as a non-limiting example, control dGH probes of a ladder can have 100, 105 and 110 oligonucleotides) and binds a control target DNA sequence whose length that differs for each control dGH probe of the ladder, for example by 1 MB, 2 MB, 3 MB, 4 MB, 5 MB, or 10 MB;
iii) each have the same number of oligonucleotides spread out evenly or unevenly across a target DNA sequence of a variable target size; for example, 10-1,000, 500, 250, 200, or 100 oligonucleotides, or 50-150 or 100 oligonucleotides, 75-100 oligonucleotides, 80-100 oligonucleotides, 85-95 oligonucleotides, or 90 oligonucleotides spread our evenly or non-evenly across within a target DNA sequence of between 5 kb and 100 kb, or 6 kb and 50 kb, or 5 kb and 10 kb, or 6 kb, 12 kb, 18 kb, or 24 kb; and/or
iv) each control dGH probe of a ladder binds to a target DNA sequence that is spaced out (e.g. by 1 MB, 2 MB, 3 MB, 4 MB, 5 MB, 10 MB, 50 MB, or 100 MB at different known distances on the control single-stranded chromatid.

Some aspects herein are directed to compositions comprising the internal control dGH probe ladders disclosed herein. Thus, control dGH probes can have any of the characteristics and properties disclosed herein for dGH probes, including that they are typically designed to be complementary to unique sequences in the genome whose chromosome is being analyzed, such as the human genome. In some embodiments, the dGH probes of the internal control dGH probe ladder have the same label. In other embodiments the set of control dGH probes that makes up an internal control dGH probe ladder have multiple colors. Some aspects herein are directed to kits comprising one or more tubes or other containers containing an internal control dGH probe ladder, which are typically premade and predesigned internal control dGH probe ladders and other containers containing any of the components provided herein for performing a dGH reaction or analyzing the results thereof. For example, such a kit can include a container/tube with a solution of nucleotide analogs or a container/tube with a set of dGH probes that are complementary to target DNA sequences on an on-test chromosome. In some embodiments, such a kit can be ordered and/or shipped together although the components may not arrive within the same box. However, in some embodiments the kit components are contained within a box that can be labeled for, and include instructions for performing a dGH assay/method.

Some embodiments of any of the aspects or embodiments herein that disclose a dGH reaction, utilize dGH paints or dGH is used to paint a chromosome. dGH paints are dGH assays that include one or more dGH probes whose target DNA sequence or combined target DNA sequence(s) span a large section/region/portion of a chromosome such as an arm, or virtually an entire or an entire chromosome. In some embodiments, one and in illustrative embodiments two or more dGH probes can be utilized to paint large segments of the chromosome for example spanning 75%, 80%, 85%, 90%, 95%, 96%, 97%, 98%, 99% or all of a chromosome or a single-stranded sister chromatid. In some embodiments, one and in illustrative embodiments two or more dGH probes can be utilized to paint at least 30%, 40%, 45%, 50%, 55%, 60%, 65%, 70%, or higher region of a chromosome or a single stranded sister chromatid. In some embodiments, dGH probes can be utilized to paint 30-100%, 40-100%, 50-100%, 30-80%, 45-75%, or 60-100% of a chromosome or a single stranded sister chromatid. In illustrative embodiments, dGH probes are utilized to paint each in one color, and preferably each with more than one color, of 2 or more, 3 or more, 4 or more, ½ of, ¾ of, most of, all but 2, all but 1 of the chromosomes, or chromatids generated therefrom, of an entire genome, such as the entire human genome. In some embodiments all chromosomes of the human genome, or all chromosomes except the sex chromosomes, or all chromosomes except the Y chromosome, are painted in more than 1 (e.g. 2, 3, 4, 5, 6, 7, 8, 9 or 10) colors. In this technique, the entire or substantially the entire genome (e.g. all chromosomes but 1) is banded using multi-color dGH assay. A non-limiting example of this is shown in FIG. 11. dGH probe sets that together bind and label all or all but 1, 2, 3, or 4 chromosomes of a human cell can be used. In some embodiments, banding patterns can range from about 2.5 Mb-10 Mb in size, although other size ranges are provided herein. The multi-color banding provides a unique spectral (e.g. fluorescent) pattern, which can be referred to as a fingerprint pattern, for each chromosome analyzed. Such fluorescent pattern can be used for to classify, detect, and/or determine structural features such as structural variations, and/or repair events of the banded chromosomes that are targeted by the dGH assay. Whole or virtually whole (all chromosomes except up to 3 chromosomes) genome banding can be performed on metaphase spreads of both diploid and haploid cells. In some embodiments, whole genome dGH paints (e.g., dGH SCREEN™, KromaTiD, Inc., Longmont, CO, USA), also referred to as dGH whole chromatid paints (e.g., dGH paints, see Table 1 for a non-limiting chromosome 2 embodiment), which are fluorescently-labelled single-stranded, unidirectional tiled oligonucleotides, for every chromosome of a genome, for example, every human chromosome (i.e., autosomes 1-22, and sex chromosomes X and Y) can be hybridized to metaphase spreads and analyzed using a fluorescence microscopy system. Thus, dGH paints, in illustrative embodiments, banded dGH paints, for example, can be a set of colors, such as 12, 11, 10, 9, 8, 7, 6, 5, 4, 3, or 2 distinct color panels such that chromosomes can be differentiated by color banding as well as size, shape, and/or centromere position.

Thus, in some embodiments, a pool of fluorescently labeled, single-stranded oligonucleotides that make up a dGH probe are tiled across some (such as, at least 30%, 40%, 50%, or higher region of a chromatid) or substantially/virtually all (such as, at least 90%, 92%, 95%, 97%, 98%, 99%, or higher region of a chromatid), or all (such as, 100%) of a chromatid. Accordingly, in some embodiments, each single-stranded oligonucleotide of a pool of single-stranded oligonucleotides that make up a dGH probe binds to one of a series of target DNA sequences, wherein the 5′ end of a target DNA sequence is the 5′ end nucleotide of a complementary DNA sequence that is closest to the 5′ end of a single-stranded chromatid that is bound by a single-stranded oligo of that dGH probe, and the 3′ end of that target DNA sequence is the 3′ end nucleotide of the complementary DNA sequence that is closest to the 3′ end of the single-stranded chromatid that is bound by a single-stranded oligo of that dGH probe.

Multi-dGH probes can be designed to identify for a wide range of target DNA sequences, depending on the application. Target DNA sequences can be between the ranges of 1 Kb and 150 Mb, 1 Kb and 100 Mb, 1 Kb and 50 Mb, 1 Kb and 30 Mb, 1 Kb and 25 Mb, 1 Kb and 10 Mb, 1 Kb and 1 Mb, 1 Kb and 100 Kb, 1 Kb and 10 Kb, 1 Kb and 5 Kb, 2 Kb and 150 Mb, 2 Kb and 100 Mb, 2 Kb and 50 Mb, 2 Kb and 30 Mb, 2 Kb and 25 Mb, 2 Kb and 10 Mb, 21 Kb and 1 Mb, 2 Kb and 100 Kb, 2 Kb and 10 Kb, 2 Kb and 5 Kb, 10 Kb and 150 Mb, 10 Kb and 100 Mb, 10 Kb and 50 Mb, 10 Kb and 30 Mb, 10 Kb and 25 Mb, 10 Kb and 10 Mb, 10 Kb and 1 Mb, 10 Kb and 100 Kb, 10 Kb and 50 Kb, 10 Kb and 25 Kb, 1 Mb and 150 Mb, 1 Mb and 100 Mb, 1 Mb and 50 Mb, 1 Mb and 30 Mb, 1 Mb and 25 Mb, 1 Mb and 10 Mb, 1 Mb and 5 Mb, 5 Mb and 150 Mb, 5 Mb and 100 Mb, 5 Mb and 50 Mb, 5 Mb and 30 Mb, 5 Mb and 25 Mb, or 5 Mb and 10 Mb. In some embodiments, the target DNA sequences bound by each of the dGH probes are consecutive target DNA sequences on one single sister chromatid, such that a multi-colored consecutive banding pattern is generated. In some embodiments, color channels selected are used to create a multi-colored banding pattern. In some embodiments, the banding pattern can be between 1 Kb and 150 Mb, 1 Kb and 100 Mb, 1 Kb and 50 Mb, 1 Kb and 30 Mb, 1 Kb and 25 Mb, 1 Kb and 10 Mb, 1 Kb and 1 Mb, 1 Kb and 100 Kb, 1 Kb and 10 Kb, 1 Kb and 5 Kb, 2 Kb and 150 Mb, 2 Kb and 100 Mb, 2 Kb and 50 Mb, 2 Kb and 30 Mb, 2 Kb and 25 Mb, 2 Kb and 10 Mb, 21 Kb and 1 Mb, 2 Kb and 100 Kb, 2 Kb and 10 Kb, 2 Kb and 5 Kb, 10 Kb and 150 Mb, 10 Kb and 100 Mb, 10 Kb and 50 Mb, 10 Kb and 30 Mb, 10 Kb and 25 Mb, 10 Kb and 10 Mb, 10 Kb and 1 Mb, 10 Kb and 100 Kb, 10 Kb and 50 Kb, 10 Kb and 25 Kb, 1 Mb and 150 Mb, 1 Mb and 100 Mb, 1 Mb and 50 Mb, 1 Mb and 30 Mb, 1 Mb and 25 Mb, 1 Mb and 10 Mb, 1 Mb and 5 Mb, 5 Mb and 150 Mb, 5 Mb and 100 Mb, 5 Mb and 50 Mb, 5 Mb and 30 Mb, 5 Mb and 25 Mb, or 5 Mb and 10 Mb. In some embodiments, the individual bands can range in size from between 1 Mb and 30 Mb, 1 Mb and 25 Mb, 1 Mb and 10 Mb, 1 Mb and 5 Mb, 5 Mb and 30 Mb, 5 Mb and 25 Mb, or 5 Mb and 10 Mb. In some embodiments disclosed herein, such as in localized banding, the banding pattern comprises bands much smaller in size. In such embodiments, bands can range in size from 1 Kb and 100 Kb, 1 Kb and 10 Kb, 2 Kb and 100 Kb, or 2 Kb and 10 Kb. In some embodiments, bands of 1 Kb (1,000 bases) in length can detected. In some embodiments, bands of 2 Kb (2,000 bases) in length can be detected.

In some embodiments, methods and compositions are disclosed herein for the detection of chromosome structural variants and repair events by labeling of one or more single-stranded chromatids with dGH probes of different colors. The hybridization pattern of the labeled dGH probes produces a fluorescence pattern, which in some embodiments is a spectral profile, which enables high-resolution detection of structural variants and repair events, facilitating distinction of benign variations from deleterious structural variations. Further, the fluorescence pattern provides information regarding complex structural variations where more than one rearrangement of chromosomal segments may have occurred.

In certain aspects, sets of labeled dGH probes can be designed to provide bands bracketing the centromere of one or more chromosome and such dGH probes can be run as a single panel of dGH probes or a plurality of (i.e., multiple) sets or panels of dGH probes for chromosome identification and enumeration. Such enumeration can be counting the number of occurrences of a target chromosome (e.g. that is a specific chromosome of the chromosomes of a genome, and/or that has a target DNA sequence, structural variation and/or repair event), or generating a list, for example a numbered list, of targeted DNA sequences, structural variations and/or repair events on a target chromosome. In certain aspects, bands on either side of the centromere of each chromosome can be labeled in different colors for further differentiation of p and q arms.

In certain aspects, sets of labeled dGH probes can be designed to provide bands which target the subtelomeric and/or telomeric regions of one or more chromosome. In some aspects, the p and q arm terminal bands of a set of dGH probes can be run as a separate panel of dGH probes or as multiple panels of dGH probes for tracking the subtelomeric and/or telomeric regions of one or more chromosome. In certain aspects, dGH probes directed to the subtelomeric and/or telomeric regions of one or more chromosomes provide structural information for the target chromosome as well as structural information for the particular arm of the target chromosome. Application of dGH probes for bands to subtelomeric and/or telomeric regions provides information for detection of structural rearrangement events involving the targeted subtelomeric and/or telomeric regions.

Any individual band may cover part or all of a gene. Also, any particular gene may be covered by all or part of one or more than one band.

In certain aspects, a target enrichment strategy may be utilized wherein additional dGH probes are utilized beyond those dGH probes used for banding, to a targeted area of interest, in order to detect features of the target area of interest. In certain aspects, the targeted area of interest may be smaller than a band. In certain aspects, the targeted area of interest may be limited to a portion of a band, cover one whole band, or span across portions of or the entirety of two or more bands. In certain aspects, dGH probes used for target enrichment can be labeled with the same or different fluorophores as the band(s) within which the target enrichment dGH probes hybridize. In aspects wherein the same fluorophore is used on the target enrichment dGH probes the intensity of the fluorescent signal is boosted in that channel. In aspects wherein a different fluorophore is used on the target enrichment dGH probes, a combinatorial fluorescent signal is produced.

In certain aspects, the dGH probes designed for target enrichment have the same or different design parameters as the dGH probes used for the banded paints. Using the same design parameters results in competitive hybridization, whereas using different design parameters results in a mixture of competitive and non-competitive hybridization. Target enrichment improves limit of detection and improves the ability to track specific chromosomal loci.

In certain aspects, methods herein can include detecting one or more target chromosomes comprising a structural feature from a population of cells, the method further comprises enumerating the structural features on the target chromosome from the population of cells. In some embodiments, the structural feature comprises one or more structural variations and/or repair events on the target chromosome. In some embodiments, the target chromosome comprising the structural feature occurs in between 1 in 1000 to 1 in 10⁸, or between 1 in 1000 and 1 in 10⁶ cells of the population of cells, or chromosomes in the population of cells. In some embodiments, wherein at least 1,000, 10,000, or 100,000 chromosomes, or between 1000 and 1×10¹⁰, 1×10⁹, 1×10⁸, 1×10⁷, 1×10⁶, 1×10⁵, or 1×10⁴ or between 10,000 and 1×10¹⁰, 1×10⁹, 1×10⁸, 1×10⁷, 1×10⁶, or 1×10⁵ chromosomes are fluorescently analyzed to identify the one or more target chromosomes comprising the structural feature. In some embodiments, methods herein further comprise before a step of contacting single-stranded sister chromatids (in some embodiments in a metaphase spread) with one or more dGH probes, contacting cells of the population of cells with a first directional genomic hybridization (dGH) probe under permeabilizing conditions such that the first dGH probe enters (i.e. penetrates cell membranes) the cells, and typically the nucleus of the cells, and wherein the dGH probes hybridize to nucleic acids typically genomic DNA within the cells, typically within the nucleus of the cells. In some embodiments, the permeabilizing conditions comprise sonication, electroporation, or contacting the cells with the first dGH probe in the presence of a transfection agent under effective conditions by which the first dGH probe enters the cells and typically the nucleus of the cells. In some embodiments, including, but not limited to methods disclosed in this paragraph, the contacting step for contacting single-stranded sister chromatids in methods herein, can further comprise contacting the one or both of the pair of single-stranded sister chromatids with a second dGH probe labeled with a second colored fluorescent label, wherein each single-stranded oligonucleotide of the second dGH probe is complementary to a portion of a second target DNA sequence on one of the single-stranded sister chromatids and comprises a second colored fluorescent label, and the detecting is detecting the first colored fluorescent label and the second colored fluorescent label thereby detecting the structural feature(s) and/or repair event(s) on the target chromosomes from the population of cells. In some embodiments of methods herein, including, but not limited to methods disclosed in this paragraph, a first dGH probe binds a target DNA sequence on the target chromosome that encompasses at least 25, 50, 75, 90, 90, 95, 96, 97, 98, or 99% of a chromosome. In some embodiments of methods herein, including, but not limited to methods disclosed in this paragraph, a first dGH probe binds a target DNA sequence on the target chromosome that encompasses at least 25, 50, 75, 90, 90, 95, 96, 97, 98, or 99% of a chromosome other than the centromeric and telomeric regions. In some embodiments of methods herein, including, but not limited to methods disclosed in this paragraph, a first dGH probe binds a target DNA sequence on the target chromosome that encompasses substantially an entire chromosome other than the centromeric and telomeric regions. In some embodiments of methods herein, including, but not limited to methods disclosed in this paragraph, a first dGH probe binds a target DNA sequence on the target chromosome that encompasses substantially an entire chromosome. In methods herein, including, but not limited to methods disclosed in this paragraph, the method can sort cells based on a fluorescence generated by dGH probe(s) binding.

In some embodiments of methods herein, at least one nucleic acid, in illustrative embodiments, one mRNA is detected or measured. In other embodiments, at least one protein is detected or measured. In some embodiments, at least one nucleic acid, for example, DNA or RNA is collected and sequenced.

In some embodiments, methods herein can include two or more dGH probes are a part of a set of dGH probes designed against a known gene sequence for a target disease. In some embodiments, the set of dGH probes are part of a screening panel for a disease type. In some embodiments, the screening panel comprises between 20 and 500, 50 and 500, 75 and 500, 100 and 500, 20 and 100, in illustrative embodiments, between 50 and 100 sets of dGH probes, depending on disease type. In some embodiments, the screening panel is a neurological disease screening panel.

Any reference spectral pattern or spectral profile may be used as a basis for comparison of the spectral profile of the chromosome under study. The reference spectral pattern or spectral profile may be that of a chromosome with a known abnormality, a chromosome considered normal, the corresponding sister chromatid, a statistically determined normal profile, a database containing reference data for chromosomes considered to have normal or abnormal profiles, or any combination thereof. In addition, the distribution of dGH probes designed against the reference genome or sequence (i.e. the density pattern of the dGH probes across unique or repetitive sequences in silico) as it relates to a reference spectral profile (increased brightness in regions with more dGH probes and reduced brightness in areas with less dGH probes) may be used to identify and describe structural variation in a test sample when a deviation in the expected spectral profile of the target(s) is present.

The pools of single stranded oligonucleotides that make up a dGH probe may be labeled by any means known in the art. Any number of different types of labels can be used to label dGH probes although typically the oligonucleotides of one dGH probe are labeled with the same label. The label on the pools of oligonucleotides can be fluorescent. The light emitted by the label on the pools of oligonucleotides can be detectable in the visible light spectrum, in the infra-red light spectrum, in the ultra-violet light spectrum, or any combination thereof. Light emitted from the dGH probes comprising the labeled oligonucleotides can be detected in a pseudo-color or otherwise assigned a color different from the actual light emitted by the pool of single-stranded oligonucleotides.

In one embodiment, a plurality of sets of dGH probes used for hybridization comprises a plurality of pools of labeled, single-stranded oligonucleotides wherein each different set of dGH probes are labeled with a different color. The plurality of sets of dGH probes may comprise differently labeled dGH probes, wherein the separate sets of dGH probes are labeled with at least two different colors (i.e. one set of dGH probes, each dGH probe comprising a pool of labeled oligonucleotides, of a first color and a second set of dGH probes, each dGH probe comprising a pool of labeled oligonucleotides, of a second color).

In some embodiments each dGH probe of a set of dGH probes are labeled with a single label and the set of multi-colored dGH probes together are labeled with two different colors, three different colors, four different colors, five different colors, six different colors, seven different colors, eight different colors, nine different colors, ten different colors, eleven different colors, twelve different colors, thirteen different colors, fourteen different colors, fifteen different colors, sixteen different colors, seventeen different colors, eighteen different colors, nineteen different colors, twenty different colors, twenty-one different colors, twenty-two different colors, twenty-three different colors, twenty-four different colors, twenty-five different colors, twenty-six different colors, twenty-seven different colors, twenty-eight different colors, twenty-nine different colors, thirty different colors, or more than thirty different colors. In some embodiments, each single-stranded sister chromatid can be assigned to a chromosome number based on the color of the set of one or more dGH probes that binds thereto and other visual features of the single-stranded sister chromatid.

In some embodiments, there can be a plurality of sets of dGH probes in the range of 1-50, 1-40, 1-30, 1-20, 1-10, 10-50, 15-45, or 20-40 sets of dGH probes. In some embodiments, there can be more than 50 sets of dGH probes. In some embodiments, the number of sets of dGH probes can depend on the total number of chromosome pairs in a subject whose chromosomes need to be analyzed. In some illustrative embodiments, there can be 24 sets of dGH probes, wherein one set binds to only one human single-stranded sister chromatids including X and Y chromosome. In some embodiments, the number of dGH probes in a particular set of dGH probes can vary, starting from at least 1 dGH probe in a set to more than 1, 10, 20, 30, 50, 75, or 100 dGH probes in a set. In some embodiments, there is at least 1 dGH probe in a set. In some embodiments, there can be 1-100, 1-90, 1-80, 1-70, 1-60, 1-50, 1-40, 1-30, 1-20, or 1-10 dGH probes in a set. In some embodiments, there can be more than 100, 200, 300, 400, or 500 probes in a set. The location of the label on the hybridizing oligonucleotides of a pool of single stranded oligonucleotides that comprise the dGH probe may be in any location on the single stranded oligonucleotide that can support attachment of a label. The single-stranded oligonucleotide may be labeled on the end of the oligonucleotide, labeled on the side of the oligonucleotide, labeled in the body of the oligonucleotide or any combination thereof. The label on the body (i.e. ‘body label) of the oligonucleotide may be on a sugar or amidite functional group of the single-stranded oligonucleotide. Typically, the body label of the oligonucleotide is bonded to the sugar backbone.

In some embodiments, methods herein can include label, at least one label, or fluorescent label selected from the group consisting of a label detectable in the visible light spectrum, a label detectable in the infra-red light spectrum, a label detectable in the ultra violet light spectrum, and any combination thereof. The wavelength of various spectrums is known to a skilled artisan. For example, a visible light spectrum generally includes light having wavelengths in the range of 400-700 nm. An infra-red light spectrum generally includes light having wavelengths in the range of 780 nm-1 mm. An ultra violet light spectrum generally includes light having wavelengths in the range of 10-400 nm.

Detection of the dGH probes may be performed by any means known in the art. Any means may be used to filter the light signal from the dGH probes, including but not limited to narrow band filters. Any means can be used to process the light signals from the dGH probes, including but not limited to computational software. In some embodiments, only certain parts of the light signature from the probes are used for analysis of chromosomal structural variants.

### Structural Variants and Repair Events

The structural variations in a genome determined by the present methods can be of any type of structural variation from a normal chromosome including, but not limited to, change in the copy number of a segment of the chromosome, an inversion, a translocation, a truncation, a sister chromatid recombination, a micronuclei formation, a chromothripsis or fragmentation event or any combination thereof. Changes in the copy number of a segment may be deletions, amplifications, or any combination thereof.

Chromosome variants can include chromosome numerical or structural variants. Chromosome variants and other outcomes of DNA replication and repair, such as sister-chromatid exchanges in a chromosome of a cell are detected on a per-cell basis across a sample or a population of cells. Chromosome structural variants and repair events included in the assessment can include some or all of those listed in Table 2, below. Thus, Table 2 provides examples of chromosome structural variants and repair events that can be detected in methods provided herein.

TABLE 2
Chromosome Structural Variants and Repair Events

```
A. Structural Variants
  a. Chromosome Numerical Variants (gain or loss of individual chromosomes)
    i. Deletions and Insertions
    ii. Total Chromosome Copy Number (genome ploidy)
  b. Translocations
    i. Unbalanced Translocations (dicentric/ acentric)
    ii. Balanced Translocations
    iii. Complex Translocations (involving 3 or more breakpoints)
    iv. Symmetrical Translocations
    v. Asymmetrical Translocations
  c. Inversions
  d. Insertions
  e. Marker Chromosomes
  f. Chromothrypsis
  g. Chromatid-Type Breaks
  h. Sister Chromatid Recombination
B. Repair Events
  a. Sister Chromatid Recombination
  b. Sister Chromatid Exchanges
```

Structural variants may be simple or complex. Simple structural variants include single occurrences of unbalanced translocations, balanced translocations, homologous translocations, inversions, duplications, insertions, and deletions. Complex structural variants include multiple simple variants in a single cell, simple variants combined with the loss or gain of genomic material, loss or gain of entire chromosomes and more general DNA damage, in illustrative the more general DNA damage variant known as chromothrypsis. Heterogeneity of variants, defined as different structural variants appearing in the genomes of individual cells of the same organism, cell culture or batch of cells can involve simple or complex structural variants. A mosaic of structural variants occurs when dividing cells spontaneously develop a structural variant and both the variant free parent and the daughter containing the variant continue to propagate.

Structural variants are distinguished from base level changes such as single nucleotide polymorphisms (SNPs) or short insertions and deletions (INDELs). Structural variants occur when the ends of multiple double strand breaks are incorrectly rejoined or mis-repaired. Depending on the subsequent reproductive viability of the cell bearing the rearrangement the consequence of a resulting structural variant can be limited to a single cell, affect a sub-set of the tissues in an organism, or if it occurs in a germ cell, may even be inherited, and affect the lineage of the organism.

The potential for DNA mis-repair that leads to chromosome structural variants including numerical variants, and/or other events such as repair events exists whenever DNA double-strand breaks (DSBs) occur. DSBs can arise endogenously during normal cellular metabolic processes, such as replication and transcription. It has been estimated that DSBs occur naturally at a rate of 50 or more per cell, per cell cycle in actively metabolizing cells, and repair occurs both during replication and through replication-independent pathways. Double strand breaks are of particular concern when induced by exogenous factors above spontaneous rates either through radiation exposure, medical interventions such as chemotherapy with certain agents, exposures to toxins or during cellular engineering processes. Of particular note are processes employed to edit or correct a genetic aberration that intentionally employ DNA double strand breaks as a step in the engineering process—such as CRISPR CAS-9. While nominally targeted, nucleases used in CRISPR processes show a measurable degree of off-target cleavage. Formation of a structural variant during an editing process requires at least two con-current double strand breaks, and since a normal human genome has two homologs of each chromosome, a single CRISPR edit can potentially have two con-current double strand breaks, the mis-repair of which would yield a translocation between the two homologs. Multiple edits, for instance a triple knock-out would have proportionally more double strand breaks and thus a proportionally larger opportunity for DSB mis-repair. The number of double strand breaks in any given cell chosen from a batch of edited cells will be a function of 1) the degree and type of editing process 2) the rate of off target editing for the given editing system 3) the degree of DSBs from active metabolism. A fourth factor, the ability of the cell to functionally repair its own DSBs can vary, and several disease states are known to detrimentally impact DNA repair.

If we then consider the normal rate of DSBs in actively metabolizing and dividing cells and the off-target nuclease cleavage, it is possible to have batches of cells with distributions of double strand breaks ranging from none (no editing, no metabolic breaks) to a maximum of 2 (number of edits)+# of off-target edits+# of con-current random DSBs. Since a structural variant requires the mis-repair of at least two double strand breaks (yielding a simple translocation or inversion), the distribution of structural variants in the above example can range from 0 (no-mis-repair) to ½ of the total number of double strand breaks.

Most DSBs are repaired by Non-Homologous End Joining (NHEJ) which operates throughout the cell cycle. In this process the broken ends are detected, processed, and ligated back together. This is an “error-prone” process because the previously existing base-pair sequence is not always restored with high fidelity. Nevertheless, this rejoining process (restitution) restores the linear continuity of the chromosome and does not lead to structural abnormalities. However, if two or more DSBs occur in close enough spatial and temporal proximity the broken end of one break-pair may mis-rejoin with an end of another break-pair, along with the same for the other two loose ends, resulting in a structural abnormality from the exchange. Examples include balanced and unbalanced translocations, inversions, or deletions. There is also a DSB repair process involving Homologous Recombination (HR) sometimes referred to as Homology Directed Repair (HDR). Homology directed repair (HDR) occurs post-replication when an identical homologous sequence becomes available and is near one another. The HDR pathway does not operate in G1 or G0 cells where the level of rad51 protein, necessary for HDR is very low or absent. However, as part of the process of gene editing (such as in the CRISPR system) the sequence to be edited is targeted and one or more DSBs are introduced to insert the desired sequence using HDR. Thus, any time DSBs are introduced, there is always a real chance that mis-rejoining among spontaneous or other DSBs form a structural variant.

Gene editing (or genome editing) is the process of intentionally modifying an organism's genome through the insertion, deletion, or replacement of DNA. Editing is dependent upon creating a double-strand break (DSB) at a particular point within the genome. This is accomplished with engineered nucleases that are targeted to specific genomic loci with guide molecules, or with sequence specifications programmed into the nuclease itself. Gene editing has been carried out with a variety of recognized methods. Widely used editing systems include CRISPR/Cas9, ZFNs, TALENs, and meganucleases. Each of these systems operate by targeting an engineered nuclease to an exact location within the genome where they bind and create sequence-specific DSBs. A target DNA sequence can be deleted, modified or replaced using the cell's endogenous repair machinery. Insertions and deletions at the edit site can range in size from a large sequence to a single base pair. Nuclease engineering, optimized delivery conditions and cellular repair mechanisms enable researchers to manipulate segments of DNA and the genes they encode for.

Editing associated errors, both on- and off-target, result in genomic variants which could impact patient safety. In order to realize the clinical potential of gene editing treatments, all editing associated errors must be identified and quantified. Editing-associated errors can be broadly classified into three categories: mis-edits, mis-repairs, and mis-edit/mis-repair combinations. Mis-edits occur when the editing enzyme creates off-target DSBs at homologous or random sites in the genome. Mis-edits typically result in small insertions or deletions (indels) of nucleotides at unintended genomic loci.

Mis-repairs occur when a cell's endogenous machinery incorrectly repairs on-target nuclease-induced DSBs. Mis-repairs result in unintended changes to the edit site that can vary from single base pair insertions/deletions to large genomic rearrangements.

Additionally, combinations of these errors can take place in which a mis-repair occurs at an off-target site. While less frequent, this can result in genomic changes that are particularly complex and difficult to identify. All editing-associated errors can result in genomic variants that are potentially harmful and represent risk for the patient. Measuring nuclease-induced changes at the edit site and throughout the genome is necessary, since it is possible for even low-frequency, heterogeneous, rearrangements to have serious consequences. Understanding the existing heterogeneity and spontaneous rate or rearrangements that exist pre-editing is essential for measuring editing effects.

Chromosomal instability (CIN) is a form of genomic instability (GIN) that involves frequent cytogenetic changes leading to changes in chromosome copy number (aneuploidy). Chromosomal instability is the predominant form of genomic instability that leads to changes in both chromosome numbers and structure. Numerical CIN is a high rate of either gain or loss of whole chromosomes, also called aneuploidy. Normal cells make errors in chromosome segregation in about 1% of cell divisions, whereas cells with CIN increase the error rate to 20% of cell divisions. By contrast, structural CIN is the rearrangement of parts of chromosomes and amplifications or deletions within a chromosome. Almost all solid tumors show CIN, and about 90% of human cancers exhibit chromosomal abnormalities and aneuploidy. The features of CIN tumor include global aneuploidy, loss of heterozygosity, homozygous deletions, translocation, and chromosomal changes such as deletions, insertions, inversions, and amplification.

A chromosome numeric variant refers to a chromosome variant having a change in the number of chromosomes, or an insertion or deletion of at least 100 kilobases in length. Thus, this change in total chromosome copy number (genome ploidy) can occur by the addition of all or part of a chromosome (aneuploidy), the loss of an entire set of chromosomes (monoploidy) or the gain of one or more complete sets of chromosomes (euploidy). Chromosome numerical aberrations may occur, involving the gain or loss of an entire chromosome. In some cases, more than one pair of homologous chromosomes may be involved. Triploidy (3N) is related to poor prognosis, particularly in cancers with higher mortality such as gastric cancer, and colon cancer. Tetraploid (4N) cells are considered important in cancer because they can display increased tumorigenicity, resistance to conventional therapies, and are believed to be precursors to whole chromosome aneuploidy. Tetraploidy and chromosomal instability (CIN) combined are a dangerous combination. By virtue of having higher P53 gene copy number, activation may inadvertently promote formation of therapy-resistant tetraploid cells. In an example, disruption of the tumor suppression gene P53, due to loss or inactivation of chromosome 17p13 is a genotoxic event that impacts tumorigenesis and leads to development of lymphoma and leukemia. Some of the most common genetic disorders are associated with chromosome number variants, such as but not limited, Down's Syndrome (trisomy 21), Edward's Syndrome (trisomy 18), Patau Syndrome (trisomy 13), Cri du chat Syndrome or 5p Minus Syndrome (partial deletion of short arm of chromosome 5), Wolf-Hirschhorn Syndrome or Deletion 4p Syndrome, Jacobsen Syndrome or 11q Deletion Disorder, Klinefelter's Syndrome (presence of an additional X chromosome in males), and Turner Syndrome (presence of only a single X chromosome in females).

A translocation occurs when a chromosome breaks and a portion of the broken chromosome reattaches to a different chromosome, thereby creating a fusion product that may lead to disease. For example, chromosomal translocations are observed in acute myeloid leukemia, where a portion of Chromosome 8 will break off and fuse with part of Chromosome 11, thereby creating an 8/11translocated product, or a fusion gene. Translocations can be balanced or unbalanced (i.e., dicentric or acentric), complex (i.e., involving three or more breakpoints), symmetrical or asymmetrical. The occurrence of translocations observed by dGH are indicative of chromosome instability.

A chromosomal inversion is a chromosome structure abnormality that can result from the misrepair of two double-stranded breaks occurring at different points along a portion of the chromosome, such that this interstitial portion of the chromosome becomes effectively rotated through 180° after a “mis-rejoining” among the broken ends of the chromosome. Importantly, this mis-rejoining must occur in such a way as to maintain the same 5′ to 3′ polarity of the strands of the chromosome and that of the inverted segment. While the backbone polarity is maintained, the DNA sequence of the nitrogenous bases within the segment is reversed. Genetic material may or may not be lost because of the chromosome breaks. A paracentric inversion occurs when both breaks occur in the same arm of the chromosome. A pericentric inversion occurs when one break occurs in the short arm and the other in the long arm of the chromosome. A chromosome 9 inversion is one of the most common structural balanced chromosomal variants and has been observed in congenital anomalies, growth retardation, infertility, recurrent pregnancy loss, and cancer. It is a particular problem to detect small inversions, such as those under 5 MB with most techniques. dGH is particularly suited to detecting these small structural variants and has been demonstrated to routinely detect inversions of below 10 kB.

Chromosomal insertions are the addition of genetic material to a chromosome. Such an insertion can be small, involving a single extra DNA base pair, or large, involving a piece of a chromosome. The effect of the insertion depends upon its location and size. For example, the insertion of one base pair could lead to a shift in the reading frame (i.e., a frameshift) during translation, resulting in synthesis of a defective protein that could lead, for example, to a birth defect. In another example, the insertion of three base pairs, though slightly larger, would not throw off the reading frame, and potentially would be less harmful than having the insertion of just one base pair. In another example, a large portion of one chromosome is inserted into another chromosome. Gain of chromosome 8q24.21 is a well-known insertion structural variant that causes the amplification of the oncogene, cMYC. Gain of this locus can increase gene expression or lead to uncontrolled activity of the onco-encoded proteins, and is observed in several cancers, including but not limited to colorectal carcinoma. It is very difficult to detect small insertions with most techniques. dGH can detect insertion 5 MB and smaller.

Chromosomal deletions, sometimes known as partial monosomies, occur when a piece or section of chromosomal material is missing. Deletions can be just a base pair, part of a gene, an entire gene, or part of the chromosome. For example, DiGeorge syndrome (22q11.2 deletion syndrome) is a disorder caused when a small part of chromosome 22 is missing. Similar to small insertions, deletions smaller than 5 MB are difficult to detect with techniques other than dGH.

A number of marker chromosomes are known and can be identified using dGH in methods herein. Iso-chromosomes are supernumerary marker chromosomes made up of two copies of the same arm of a chromosome. The presence of an isochromosome in addition to the normal chromosome pair leads to a tetrasomy of the arm involved. The accurate description of such a marker chromosome using only conventional cytogenetic techniques is often difficult. Illustrative methods herein utilize dGH to identify marker chromosomes.

A marker chromosome is a small fragment of a chromosome that is distinctive, that is present in a cell as a separate structure from the rest of the chromosomes, and generally cannot be identified without specialized genomic analysis due to the size of the fragment. The significance of a marker is variable as it depends on what material is contained within the marker. A marker can be composed of inactive genetic material and have little or no effect, or it can carry active genes and cause genetic conditions such as iso(12p), which is associated with Pallister-Killian syndrome, and iso(18p), which is associated with mental retardation and syndromic facies. Chromosome 15 has been observed to contribute to a high number of marker chromosomes, but the reason has not been determined.

Chromothrypsis is a process by which dozens to up to thousands of chromosomal rearrangements occur in localized regions of one or a few chromosomes. When chromothrypsis occurs, essentially one or a few chromosomes (or a chromosome arm) is shattered, leading to the simultaneous creation of many double strand breaks. Most of the shattered fragments are stitched back together though Non-Homologous End Joining (NHEJ), which leads to the creation of a chromosome with complex, highly localized chromosomal rearrangements (e.g., chromoanagenesis). Broken DNA fragments may also be joined together to form circular, extrachromosomal double minute chromosomes. Chromothrypsis has been observed in the development of cancers. For example, de novo rearrangements caused by chromothrypsis can trigger chromosome instability in subsequent cell divisions.

Chromatid-type breaks refers to a break in the chromosome, where the break and rejoining affect only one of the sister-chromatids at any one locus. This differs from “chromosome-type” breaks, where the breaks and re-joins always affect both sister-chromatids at any one locus. Unrepaired DNA strand breaks contribute to genomic instability. Unrepaired chromatid breaks representing DNA strand breaks can result in chromosome deletions, translocations and gene amplifications seen in human cancers.

Sister chromatid recombination (SCR) is a normal repair event that can also result in a structural variation that occurs during meiosis and promotes genomic integrity among cells and tissues through double-strand break repair. SCR refers to the homologous recombination process involving identical sister chromatids that results in a uni-directional non-crossover event, otherwise known as a gene conversion event. It is thought to occur when the homologous recombination intermediate known as the double Holliday junction is resolved in such a way that it results in a non-crossover. SCR can be employed by the cell to resolve both single-stranded DNA lesions (which involve a corresponding replication fork collapse) and double-stranded breaks. Gene conversion between sister chromatids is not usually associated with reciprocal exchange and is differentiated from an SCE for that reason. Aberrant SCR is associated with congenital defects and recurrent structural abnormalities. Mutations affecting genes involved in SCR have been linked to infertility and cancer. SCR is associated with chromosome instability, particularly with large structural rearrangements, aneuploidies and infertility. It is important to note that SCEs are detected by dGH but missed in all other karyotype assessment methods.

A number of complex events, such as repair events, produce structural variants that are listed in Table 2. Complex events produce complex chromosomal rearrangements (CCR) or complex genomic structural rearrangements that involve at least two chromosomes and three breakpoints with varied outcomes, except for simple or 3-break insertions. These CCRs may involve distal segments causing reciprocal translocation, or interstitial segments leading to insertion, inversion, deletion, or duplication, or they may involve a combination of both distal and interstitial segments. One chromosome may also have more than one aberration such as an inversion and a translocation that can coexist on the same chromosome.

The structural variants include micronuclei, chromosome fragments, extra-chromosomal DNA (i.e., ecDNA), multi-radial chromosomes, iso-chromosomes, chromoplexy, rings, centromere abnormalities and chromosome condensation defects. Several of these structural variants arise due to defects in the normal metabolism of the chromosomal DNA. These structures are described in greater detail in the paragraphs below.

Micronuclei (MN) are extra-nuclear bodies that contain damaged chromosome fragments and/or whole chromosomes that were not incorporated into the nucleus after cell division. Micronuclei can be induced by defects in the cell repair machinery and accumulation of DNA damages and chromosomal aberrations. A variety of genotoxic agents may induce micronuclei formation leading to cell death, genomic instability, or cancer development.

Multi-radial chromosomes are complex aberrant chromosomal structures that appear, in karyotype analysis, as a fusion of more than two sister chromatids, and are a hallmark of chromosomal instability. Multi-radial chromosomes are observed in several cancer predisposition syndromes, including Ataxia Telangiectasia, Nijmegen Breakage Syndrome, Bloom Syndrome, Werner Syndrome and Fanconi Anemia.

Extra-Chromosomal DNA (ecDNA) is any DNA found outside the chromosomes. In certain cases, ecDNA can be deleterious and can carry amplified oncogenes. In some aspects, deleterious ecDNA can be 100-1,000 times larger than kilobase size circular DNA found in healthy somatic tissues. In certain aspects, ecDNA includes episomal DNA and vector-incorporated DNA. ecDNA amplification promotes intratumoral genetic heterogeneity and accelerated tumor evolution. For example, ecDNA amplification has been observed in many cancer types but not in blood or normal tissue. Some of the most common recurrent oncogene amplifications have been observed on ecDNA. EcDNA amplifications resulted in higher levels of oncogene transcription compared to copy number-matched linear DNA, coupled with enhanced chromatin accessibility, and more frequently resulted in transcript fusions. Patients whose cancers carried ecDNA had significantly shorter survival, even when controlled for tissue type, than patients whose cancers were not driven by ecDNA-based oncogene amplification.

Chromosomal fragmentation occurs when the condensed chromosomes are rapidly degraded during metaphase, and results in cell death. Chromosome fragmentation is a major form of mitotic cell death which is identifiable during common cytogenetic analysis by its unique phenotype of progressively degraded chromosomes. Chromosome fragmentation is a non-apoptotic form of mitotic cell death and is observed from an array of cell lines and patient tissues. Its occurrence is associated with various drug treatment or pathological conditions.

Chromoplexy is a complex DNA rearrangement, wherein multiple strands of DNA are broken and ligated to each other in a new configuration, effectively scrambling the genetic material from one or more chromosomes. Chromoplexy often involves segments of DNA from multiple chromosomes (e.g., five or more). In one example of chromoplexy, homologous repeated sequences (i.e., HSRs) may become expanded by homologous recombination events in which a break induced in a palindromic sequence promotes homologous strand invasion and repair synthesis. Chromoplexy can account for many of the known genomic alterations found in prostate cancer by generation of oncogenic fusion genes (e.g., BRAF and MAPK1 fusion) as well as by disruption or deletion of genes located near rearrangement breakpoints (e.g., tumor suppressor genes PTEN, NKX3.1, TP53, and CDKN1B).

Ring structures are circular chromosomal DNA that, in some instances, result from two terminal breaks in both chromosome arms, of a chromosome followed by fusion of the broken ends, or from the union of one broken chromosome end with the opposite telomere region, leading to the loss of genetic material. Alternatively, rings can be formed by fusion of subtelomeric sequences or telomere-telomere fusion with no deletion, resulting in complete ring chromosomes. Ring chromosomes may be dicentric (i.e., with more than one centromere) or acentric (i.e., no centromere). Ring chromosomes are associated with a variety of genetic diseases. In one example, r(20) syndrome is a rare genetic disorder characterized by a ring chromosome 20 replacing a normal chromosome 20.

Centromere abnormalities, such as “spindling,” are aberrant chromosome rearrangements, such as from SCE or SCR, of long tandem DNA sequences at the centromere that can lead to chromosome fusions and genetic abnormalities. In some instances, intrachromatid recombination occurs, leading to the formation of a circle, such as a ring, and a deletion of a portion of the chromatid. In other instances, recombination leads to unequal exchange, thereby introducing instability in the total size of the centromeric array. In still other instances, homologous recombination at identical centromere sequences between different chromosomes can lead to the formation of dicentric and acentric chromosomes (i.e., two centromeres and no centromere, respectively). Chromosomal structural variants due to centromere abnormalities have been observed in a wide variety of cancers, including but not limited to breast cancer (chromosomes 12, 8, 7), colorectal cancer (chromosome 18), pancreatic cancer (chromosomes 18, 8), and melanomas (chromosomes 1, 18). Chromosome condensation defects are defects in the reorganization or compaction of the chromatin strands into compact short chromosome structures that occurs in mitosis and meiosis. Generally, defects in chromosome condensation are caused by defects in one or more of the structures in is mediated by the condensin complex and other proteins and is necessary to prevent chromosomes from being entangled during chromosome segregation. In an example, Gulf War Illness (GWI) impacts 25-30% of gulf war veterans and is associated with a variety of condensation defects.

Sister chromatid exchanges (SCE) are error-free swapping or cross-over event involving precisely matched and identical DNA strands of the sister chromatids of a condensed chromosome during mitosis. SCE while not structural variants, are associated with elevated rates of genomic instability due to an increased probability that alternative template sites such as repetitive elements adjacent to the break site will produce an unequal exchange resulting a structural variant. SCE frequency is a commonly used index of chromosomal stability in response to environmental or genetic mutagens. A wide range of human diseases have been linked to SCE, including but not limited to lung cancer, leukemias, hearing loss, thyroid tumors, xeroderma pigmentosum and diffuse gastric cancer. An advantage of dGH, and especially multi-color dGH is the ability to detect SCEs.

Although SCE events are not in themselves structural variants, they can be used as an indicator of chromosomal instability (D Pascalis et al, 2015). SCE levels are increased in patients with various cancers associated with genomic instability (Salawu et al., 2018; Soca-Chafre et al., 2019; Xu et al., 2015). Unlike translocations, inversions, and ring structures that are produced via NHEJ-mediated mis-joining of DSBs, SCEs arise during DNA replication and require HDR (Wilson and Thompson 2007). SCEs are non-recurrent repair events that appear as a random distribution within a population, while inversions, as true structural rearrangements, are stable and are passed on to daughter cells over many cell generations (i.e., they are recurrent within a population). While dGH can distinguish between recurrent and non-recurrent repair events in a population of cells, localized dGH assays can be helpful to identify these repair events as true inversions or SCEs. Other proxies of genomic instability, such as chromatid breaks and gaps, can arise only as a result of an event that occurred during the cell cycle immediately prior to the mitosis where it is observed.

The methods disclosed herein may be practiced in combination with other techniques for detecting chromosomal abnormalities. In one embodiment, the methods disclosed herein may be practiced in combination with chromosomal staining techniques, including but not limited to staining of chromosomes with DAPI, Hoechst 33258, actinomycin D or any combination thereof.

Directional genomic hybridization (dGH) is a technique that can be applied to measure both the rates of mis-repair and the identity of certain mis-repairs. This method can be employed to detect both de novo SVs in metaphase chromosomes in individual cells or can be utilized to assess SVs involving a particular genomic locus. In previous embodiments, the detection of orientation changes (inversions) sister chromatid exchanges and non-crossover sister chromatid recombination as well as a balanced allelic translocation would be visualized as the same signal pattern change in a single cell with a single method. These SVs are detected alongside and in addition to the SVs visible to standard chromosome-based cytogenetic methods of analysis (unbalanced and balanced non-allelic translocations, changes in ploidy, large inversions, large insertions, and large duplications). However, unless targeted methods are employed, differentiating the orientation change SVs (high risk) from transient repair intermediates resulting from SCE and SCR events (low risk), and balanced translocations between two homologous chromosomes (relatively low risk) is often not possible. In recent years, additional types of mis-repairs and their relative contribution to oncogenesis and genomic instability have been described, further illustrating the need for more precise resolution of the events visible via dGH, beyond the obvious need for a more precise mapping of the breakpoints and account of genomic regions involved in SVs detected by dGH. Most of the work discussed here on molecular mechanisms of SCE formation involves studies in yeast and is much further along than our knowledge for mammals. While we do not claim the mechanisms are identical, to the extent processes are similar, the approaches described in the present application will help further such knowledge

Because DNA mis-repair can lead to cell death or pose a risk to patients, novel techniques to both measure rates of mis-repair and provide hypothesis free, de novo identification of SVs are essential. The present disclosure combines dGH methods with unique dGH hybridization dGH probe designs and unique image analysis methodologies to provide identification and characterization of SVs with markedly increased resolution. Because this characterization includes location and orientation data, it can be combined with publicly available bioinformatic data about which genes, promotors and genomic regions to assess the risk of genotoxicity caused by the mis-repair or mis-repairs to individual cells as well as with proteome and transcriptome data to inform patient diagnosis.

Directional genomic hybridization (dGH) can be performed as either a de novo method which can detect structural variants against a reference (normal) genome or as a targeted (i.e. localized) method, assessing structural variants at a particular target region such as an edit site (FIG. 5A-FIG. 5D). In both embodiments, the dGH method is designed to be qualitative and provides definitive data on the prevalence or occurrence of one or more structural variants in individual cells. When using the targeted embodiment, the presence of a specific target can be inferred, as the assay is designed as a binary test for the target.

However, the de novo embodiment, while able to detect an SV without prior target hypothesis, (e.g., a putative telomeric inversion of the p arm of C3, of approximately 7 Mb) typically does not provide as precise information regarding size, location or sequence of the variant.

Banding chromosomes via differential staining of light and dark bands or multi-colored bands is a technique widely employed for distinguishing a normal karyotype from a structurally rearranged karyotype. Each method of banding has its strengths and weaknesses. G-banding and inverted (or R-banding with DAPI) and chromomycin staining are the most broadly used techniques for producing differential light and dark banding of chromosomes and are adequate for detecting a subset of simple structural variants 62 including numerical variants (variations in the number of whole chromosomes or large parts of chromosomes), simple translocations, and some large inversions (depending on the degree of band pattern disruption). They are rapid and cost-effective DNA-staining methods and are the current industry standard for karyotyping in clinical diagnostics. Though they provide basic karyotype information, these techniques have very limited utility for detecting smaller numerical variants (deletions and insertions) and small inversions, and often cannot be used to describe complex rearrangements. They do not provide any locus-specific information other than to describe an observed light/dark band disruption involving the general region of interest. In the case of translocations, they also have significant blind spots. If chromosome banding patterns present as alternating “ . . . light-dark-light-dark . . . ” sequences, as in G-banding, the resolution of exchange breakpoint locations will be inherently inferior to the same pattern presenting as alternating color sequences, say, “ . . . R-G-B-Y . . . ”. These staining-based methods are subject to “Three-band Uncertainty” in localization of translocation breakpoints (Savage 1977) that applies to the first (light-dark) situation. In addition, these methods do not detect balanced translocations that are equivalent exchanges between two homologous chromosomes with breakpoints at the same loci or nearby loci, nor will they detect sister chromatid exchanges/sister chromatid recombination (gene conversion) events.

Whole chromosome FISH painting techniques such as SKY and MFISH can be used to provide a more precise description of observed structural variants, because each chromosome (2 copies of each chromosome per normal cell) is labeled in a different color. These techniques identify which chromosomes are involved in an observed rearrangement, but they cannot provide breakpoint coordinates nor identify the genomic segments of the chromosomes included or missing as a product of the rearrangement. For example, much like with the monochrome dGH paints, a deletion or an amplification cannot be attributed to any particular region or locus of a specific chromosome via SKY, MFISH, or similar methods.

Band-specific multicolor labeling strategies (the most well-known method is mBAND) can provide a more resolved picture of certain complex events, including identification of which segments of a particular chromosome are involved in a rearrangement, limited to the resolution of the assay. The resolution of the mBAND assay is determined by how discreet (small) the band size is in any given region, and how suitable the sample is for resolving the bands both for their presence, and their relative order (e.g., how long and stretched out the chromosomes are). But like all the other FISH-based techniques, mBAND cannot detect balanced translocations between homologous chromosomes, small inversions, or sister chromatid exchange/sister chromatid recombination events (gene conversion) events, no matter how high the resolution is. Furthermore, the bands are created by amplifying and differentially labeling portions of needle micro-dissected chromosomes through DOP-PCR to create overlapping libraries of probes, and assessing these bands in a normal karyotype against high-resolution G-banding and/or inverted DAPI-banding in order to deduce the position of each band. Therefore, the precise start and end coordinates of each band are unknown, and can only be inferred by comparison to the highest resolution G-banding of metaphase cells with a normal karyotype.

“Oligopainting” as referred to in the U.S. Patent Application Publication No. 2010/0304994, would have an advantage over mBAND in that the bands could be precisely designed against known genomic coordinates with synthetic oligos. The precise start and end of each band would be known genomic coordinates, and not an estimation based on comparison to light-dark banding on a normal karyotype. But like all the other FISH-based techniques, “oligopainting” would not be able to detect balanced translocations between homologous chromosomes, small inversions, or sister chromatid exchange/sister chromatid recombination events (gene conversion) events.

The presently disclosed methods for detecting structural variations provide the missing elements from the monochrome dGH paints: providing specific genomic coordinates, and differentiating true inversion events (which involve a re-ordering of the genomic segments) from sister chromatid exchange events (which do not change the order of genomic segments, but which cannot be differentiated from inversions using the monochrome dGH paints). The risk associated with these 2 events (inversions are high risk, SCEs are low risk because they are essentially a “correct repair” and does not result in a change in order or copy number of genomic segments) is important for clinicians to understand. There is a risk for a loss of heterozygosity (one good copy of a gene is replaced with the bad copy—resulting in a disease phenotype) associated with sister chromatid exchange, but it should be distinguished from true inversion events in the context of risk and patient outcomes. CRISPR Cas9 and other gene editing systems which rely on DNA breaks and DNA break repair need accurate risk profiles. Differentiating these SCE/SCR “false positives” from potentially genotoxic events (inversions) is possible with the presently disclosed methods. The order of the genomic segments is visible, as well as the orientation of the signal on either the primary sister chromatid or the opposite sister chromatid (see schematics). K-Band is differentiated as a technique from the other multi-colored banding methods because of the sample preparation method required, which involves the removal of the newly synthesized DNA daughter strand from a sister chromatid complex, providing a single stranded template that allows for chromatid-specific labeling.

In the context of gene editing, the detection and identification of structural variants produced during the manipulation and alteration of a genome is a priority for patient health. The need to measure inversions and sister chromatid exchanges as a significant piece of the repair equation alongside deletions, amplifications, and translocations at a high resolution in single cells is widely recognized by the diagnostics community as a need—as well as among regulators. The presently disclosed methods are able to deliver structural variant data that is missed by sequencing and inaccessible using other differential banding or FISH based banding methods. As outlined in the previous description, the sample preparation component of the assay in combination with the uni-directionality of the oligo probes fluorescently labeled single-stranded oligonucleotides enables an assessment of events that are not detectable by other banding techniques and provide and important additional level of structural variant data. Because enzyme-directed gene editing processes hijack and harness cellular synthesis and repair machinery they introduce a level of additional complexity to an otherwise very complex process. Sequencing approaches for confirming the edit, as well as for assessing the rest of the genome for un-intended effects frequently rely on the presence of an intact target sequence to generate data. However, if a resection and deletion has occurred in the region of the target sequence then amplification of the region for sequence analysis is not possible. And in a pooled DNA format, this information will be missing—which is a concern when screening for structural variations that include copy number variation and carry an increased risk for genotoxicity. Complex structural variants are also very difficult to assess via sequencing. In this way, the most genomically unstable and dangerous structural variants are the most likely to be missed by sequencing. In the context of a metaphase spread, the entire genome of each cell is available to be measured and assessed for the presence of structural variation without any amplification and sequence analysis. De novo rearrangements as well as rearrangements to the target of interest can be measured, and populations of edited cells can be monitored over time for both unintentional spontaneous and stable structural changes that could be of concern (like cancer-driving fusion genes) as well as the stability of the desired edit over time. With the genomic coordinate specifics offered by the presently disclosed methods, sequencing can be employed to take a deeper base-pair specific look at the structural variants observed. The two techniques can be used in concert to enable more precise detection and characterization of an edited genome.

### Analysis of Extrachromosomal DNA (ECDNA)

Biological samples comprising the DNA of cells are prepared to facilitate contacting the sample with dGH probes comprising a pool of single-stranded oligonucleotides, each oligonucleotide being unique and complementary to at least a portion of the DNA. In certain aspects, the biological sample comprising cellular DNA further comprises ECDNA. Both the ECDNA and the chromosomal DNA can be hybridized with dGH probes having the same nucleic acid sequences and fluorescent light signatures. In aspects where ECDNA and chromosomal DNA are similarly labeled, a determination can be made from where on the chromosome the ECDNA originated.

dGH probes used for banding a chromosome under examination can be selected to specifically locate the chromosomal source or origination of DNA found in ECDNA. In certain aspects, spectral analysis of the hybridization pattern of a pool of labeled, single-stranded oligonucleotides to chromosomal DNA allows for identification of the chromosomal source of DNA in the ECDNA. The comparison of spectral signatures, in certain aspects the investigation of similarities in spectral signatures, between chromosomes and ECDNA provides for identification of particular chromosomal DNA as the source of amplified regions of DNA incorporated in ECDNA. In certain aspects, the analysis of banding patterns resulting from hybridization of a pool of labeled, single-stranded oligonucleotides to chromosomal DNA provides for identification of genes and regions of interest in the chromosome under study. In certain aspects, a band or bands identified as of interest in chromosomes under study can then be used to inform the design of a specific dGH probe or panels of dGH probes if multiple bands are identified as source material incorporated into ECDNA to further characterize sequences present in the ECDNA.

Methods for analysis for ECDNA can be applied to episomal DNA, vector-incorporated DNA as well as any other DNA within a cell which is not present on a chromosome.

### Spectral Analysis

Methods provided herein that include performing spectral analysis to detect at least one structural feature, such as a structural variation, and/or to detect a repair event in a chromosome from a cell or for identifying a chromosome that is the chromosomal source of extrachromosomal DNA (ECDNA) in a cell, in certain illustrative embodiments include detecting and analyzing spectral information, such as fluorescence images or measurements made therefrom, produced upon excitation of fluorescence labels and/or dyes by a light source. Such labels and/or dyes are found on probes and/or DNA stains associated with chromatids, which typically are analyzed on metaphase spreads in methods herein. Such labels and/or dyes can be detected in the visible, infrared or ultraviolet spectrum of light. Color channels can be selected to detect specific regions of the light spectrum based on the fluorescence labels and/or dyes selected. Thus, during a dGH analysis a hybridization pattern is generated upon binding of at least 2 dGH probes to one or both single-stranded sister chromatids that are under analysis. This hybridization pattern is used to generate one or more spectral measurements upon excitation of the labels on the hybridized dGH probes typically to generate a spectral pattern, which in illustrative embodiments is a fluorescent pattern. Typically, this is performed using a fluorescence microscope and analysis software, which generates a spectral image representing the hybridization pattern. In some embodiments, spectral measurements can include fluorescent wavelength intensities of the labels on the hybridized probes. In some embodiments, spectral measurements can include relative fluorescent units (RFU) of the labels on the hybridized probes. In some embodiments, spectral measurements can include representation of oligo density distribution across a chromosome. In some embodiments, spectral measurements can be a collection of different data points on fluorescent wavelength intensities, and RFU. In some embodiments, spectral measurements can include any form of comparison, such as, but not limiting to overlaying of one or more data points across fluorescent wavelength intensities, oligo density distribution, and/or a chromosome image, such as, but not limiting to an ideogram. In certain illustrative embodiments, spectral measurements can include overlaying wavelength intensities of the labels on hybridized probes with an oligo density distribution. In certain illustrative embodiments, spectral measurements can include overlaying wavelength intensities of the labels on hybridized probes with an oligo density distribution, and a chromosome image. Furthermore, such analysis can include overlaying markers used to detect repeat sequences over any of the multi-color dGH fluorescence information. This layering of various sources of information increases the ability to detect, determine, and classify repair events and/or structural features such as structural variations. Furthermore, this layering of these various sources of information can be combined with methods herein to narrow down the chromosomal region of a particular structural feature or repair event.

In some embodiments of any embodiments or aspects that include spectral measurements, size of the band produced by the hybridizing dGH probes can be determined. In some embodiments, spectral measurements form a banding pattern comprising bands of different colors, and each color refers to the wavelength of light emission that can be detected as a separate and distinct wavelength. In such embodiments, the bands can be as small as 1,000 bases. In some embodiments, bands can be as small as 2,000 bases. In some embodiments, spectral measurements can include spectral intensity measurements. In such embodiments, spectral intensity measurements are along one or both sister chromatids. In some embodiments, spectral intensity measurements can be used to create a spectral fingerprint of one or both of the sister chromatids. In some embodiments, spectral measurements of one or both sister chromatids can be compared to a reference spectral measurement. In some embodiments, reference spectral measurements can include spectral intensity measurements, and in some embodiments, such reference spectral intensity measurements can be used to normalize spectral intensity measurements of one or both sister chromatids under study.

In some embodiments, spectral measurements can be used to form a spectral pattern (e.g., a spectral profile). A spectral pattern (e.g., spectral profile) can be understood as a collection of data layers that can effectively assist in detecting at least one structural feature, such as structural variation, and/or repair event in a chromosome from a cell or for identifying a chromosome that is the chromosomal source of extrachromosomal DNA (ECDNA) in a cell as disclosed herein. Obtaining a spectral profile of a particular chromosome can comprise: (a) detecting fluorophores by methods not limiting to staining a chromosome or a portion thereof, or any technique that involve staining a DNA; (b) detecting hybridization of probes that can be achieved by detecting signals from a specific color channel and combining it with data on chromosome location; (c) integrating the signal from total fluorophores across a chromosome, or a portion thereof to form a fluorescence pattern, for example to form a fingerprint pattern of the chromosome, or a portion thereof; and (d) obtaining a profile of the fluorophores from the fluorescence pattern (e.g. fingerprint pattern).

A spectral pattern, for example a fluorescence pattern, such as a spectral profile, for example a fluorescence profile, can include the variation of light intensity at a given wavelength. As such, the fluorescence pattern (e.g. spectral profile) of the fluorescently labeled spectral image can represent a banding pattern based on hybridization of differently colored dGH probes to one or more sister chromatids. In such embodiments, the fluorescence pattern (e.g. spectral profile) represents a banding pattern comprising bands of different colors.

In certain illustrative embodiments, spectral analysis captures information about all fluorophores and/or stains in one microscopic image. Such enhanced digitized version can be enhanced for example, such that different colors generated by the microscopic analysis are more apparent and/or appear as different colors in the digitized image. As understood by those of ordinary skill in the art, spectral measurements can be obtained and analyzed by any number of methods including, but not limited to fluorescence microscopy, laser scanning microscopy, fluorescence cytometry, analysis software, or other fluorescence analyzers, and any combination thereof. In some embodiments, a scanning microscope system (e.g., ASI scanning microscope system (City, state) and analysis software, such as cytogenetics software (e.g. GenASIS cytogenetics software) can be used for imaging and analysis such that a spectral profile is generated from hybridized dGH probes and is analyzed to detect one or more chromosomal variants and/or repair events, such as SCEs. In some embodiments, spectral profiles of single-stranded sister chromatids generated from target chromosomes or chromosome pairs from on-test cells, can be selected for analysis from metaphase spreads and compared to spectral profiles of corresponding control target chromosomes or chromosome pairs. -In some embodiments, due to the close proximity of bands to each other, adjacent bands appear to bleed over into each other. The bleeding over can be used as an additional marker to improve localization of events within a band based on the presence of bleed over from adjacent bands and the ratio of bleed over signal to band signal.

Directional Genomic Hybridization (dGH) Expansion.

In certain aspects, expansion microscopy (Asano et al. (2018) Current Protocols in Cell Biology e56, Volume 80) can be applied to dGH samples to improve the spatial resolution of dGH. In certain aspects, expansion microscopy involves embedding a sample in a swellable hydrogel, then chemically linking the sample to the hydrogel. The sample can then be labelled, swelled, and imaged. The process of swelling the sample increases the spatial (x, y, z) resolution to levels comparable to confocal or super resolution fluorescence imaging on a non-expanded sample. Accordingly, improved ability to localize events, for example structural variations, is achieved.

### Nodal Analysis

Methods are disclosed herein for identifying one or more structural features of a subject DNA strand. In certain aspects, such methods are implemented in a processor. In one aspect, methods for identifying one or more structural features comprise receiving spectral measurements representing at least one sequence of base pairs on a subject DNA strand, the spectral measurement can include frequency data corresponding to the sequence of bases of the subject DNA strand. The frequency data can be divided into at least two color channels. In different aspects, various data are contained in the color channels, including but not limited to positional data and intensity data. A spectral pattern (e.g., spectral profile) can be created from such data and be converted into a data table comprising positional data, intensity data as well as other data determined to be of interest in the at least two color channels. A data table thus produced for a subject DNA strand can be compared with a reference feature lookup table comprising one or more feature nodes representing normal and/or abnormal features of a corresponding control DNA strand to identify one or more normal and/or abnormal features of the subject DNA strand. In one aspect, the feature node is defined by a color band representing a sub-sequence of bases of the control DNA strand beginning at a start base and ending at an end base.

Nodal analysis, wherein spectral pattern (e.g., spectral profile) information of subject DNA sequences is converted to numeric form for comparison to control or references DNA sequences can be performed in conjunction with the directional genomic hybridization methods disclosed herein or can be utilized in the context of other methods which provide polynucleotide sequence data convertible to a numeric form. In certain aspects, the reference or control lookup tables are a single table of values or multiple tables of values. In some aspects, the different reference or control look up tables provide values which correspond to different genomic regions. In certain aspects, the comparison of the lookup tables from the subject DNA with the reference or control look up tables is performed by a machine learning and/or AI algorithm. The values of spectral pattern (e.g., spectral profile) data from subject DNA strands can be related to specific nodes through analysis of control or reference lookup tables. A set of nodes can then be run through nodal analysis to find related pathways or effected pathways, wherein relationships between nodes are previously known or determined by analysis.

In certain aspects, the spectral pattern (e.g. spectral profile) data from a subject DNA strand can be stored to a memory for later comparison and analysis to determine structural features of interest. In some aspects, the spectral pattern (e.g., spectral profile data can be stored in a relational database, graph database, lookup tables, or any other bioinformatics database format.

In some aspects, features of interest on a subject DNA strand can be characterized as normal features which correspond to features on a healthy control DNA strand. In some aspects, features of interest on a subject DNA strand can be characterized as abnormal features which correspond to features on a reference DNA strand representing at least one abnormality.

In certain aspects, spectral pattern (e.g., spectral profile) data is analyzed from DNA regions which are not spatially collocated. In some aspects, spectral pattern (e.g., spectral profile) data originate from DNA regions in spatial proximity. In certain aspects, spectral pattern (e.g., spectral profile) data is linked by a series of keys based on oligonucleotide sequences of the pool of oligonucleotides in a dGH probe, spectrum, oligonucleotide density, chromosome, chromosome arm, band ID, band orientation, and band coverage (e.g., gene region). In some aspects, genomic features can be defined by band, band spectrum, band sequence, band orientation, and band nearest neighbors or by dGH probe, dGH probe spectrum, dGH probe orientation and dGH probe nearest neighbors.

In certain aspects, a sequence across a feature, a chromosome arm, or a chromosome can be defined by beginning at the 5′ end on one of the plurality of single stranded oligonucleotides that comprise a dGH probe, band, or region of interest, then analyzing the band spectrum, size, and coverage of each band consecutively moving toward the 3′ end. In some aspects, these features are converted into keys which can be compared against a database to determine the location and features of an aberration or abnormality and, by extension, which nodes in the database are affected by those aberrations or abnormalities. Some combinations of aberrations or abnormalities indicate specific rearrangement events, e.g., a truncated band in one region combined with extra signal of the same spectrum in a different region would indicate a translocation event.

Spectral patterns (e.g. spectral profile) data can be analyzed or meta-analyzed with any statistical analysis tools including but not limited to: graph theory, nodal analysis, artificial intelligence, machine learning (including k-nearest neighbor, principal component analysis, etc.), and neural networks.

The methods disclosed herein can be combined with methods incorporating multiple types of data into a database for analysis. In certain aspects, data from other sources includes but is not limited to sequencing, genomics, transcriptomics, proteomics, and metabolomics. In certain aspects, inversions, sister chromatid exchanges, and other dGH specific data are analyzed against sequencing data. Comparison can be performed against known, published sequencing data or against novel or unpublished data.

In some aspects, data generated by the methods disclosed herein are summarized on a report with automatically generated ideograms showing unique and recurring rearrangements and analysis, meta-analysis, or nodal analysis on both a sample level and a cohort or experiment level.

### Localized Analysis

In some embodiments, a cell analyzed in a method herein, is from a test population of cells, or a test population of cells or chromosomes are analyzed as part of a method herein. The test population of cells, and its individual cells, can comprise genetically modified cells having a recombinant nucleic acid insert. In some embodiments, the recombinant nucleic acid insert comprises a chimeric antigen receptor sequence, a transgenic sequence, a gene-edited sequence, a deleted gene sequence, an inserted gene sequence, a DNA sequence for binding guide RNA, a transcription activator-like effector binding sequence, or a zinc finger binding sequence. In some embodiments, the recombinant nucleic acid insert comprises a transgene. In some embodiments, the transgene is a chimeric antigen receptor sequence. In some embodiments, the transgene is a gene-edited sequence. In some embodiments, the transgene is a gene-edited sequence. In some embodiments, a set of multi-color dGH probes is used wherein at least one target DNA sequence for a probe of the set includes a target site for gene editing.

In certain embodiments, a dGH probe or in illustrative embodiments a set of multi-colored dGH probes, or a plurality of such sets, can be designed to target loci within a genome which are known to influence or cause a disease state. In certain embodiments, a dGH probe or in illustrative embodiments a set of multi-colored dGH probes can be designed to target genes known to be associated with the development or presence of lung cancer. Similarly, a dGH probe or in illustrative embodiments a set of multi-colored dGH probes can be designed and utilized with the methods disclosed herein for any disease or condition of interest.

In certain embodiments, a dGH probe or in illustrative embodiments a set of multi-colored dGH probes, or a plurality of such sets, can be designed to target loci within a genome which are known to be correlated with different states of a particular disease. In certain embodiments, a dGH probe or in illustrative embodiments a set of multi-colored dGH probes can be designed to target loci within a genome which are known to be correlated with genetic disorders. In one aspect, a set of multi-colored dGH probes or a plurality of probe sets can be designed as a prenatal diagnostic tool for genetic disorders.

In certain embodiments, a dGH probe or in illustrative embodiments a set of multi-colored dGH probes, or a plurality of such probe sets can be designed to target loci within a genome to provide diagnostic tools for any disease or health condition of interest. In certain aspects, the disease or condition may be selected from diseases of the respiratory tract, musculoskeletal disorders, neurological disorders, diseases of the skin, diseases of the gastrointestinal tract and various types of cancers.

In certain embodiments, a dGH probe or in illustrative embodiments a set of multi-colored dGH probes or a plurality of probe sets can be designed to target specific classes of genes within a genome. In one aspect, a dGH probe or in illustrative embodiments a set of multi-colored probes can be designed to target genes for different types of kinases.

Gene editing (or genome editing) is the process of intentionally modifying an organism's genome through the insertion, deletion, or replacement of DNA. Editing is dependent upon creating a double-strand break (DSB) at a particular point within the genome. This is accomplished with engineered nucleases that are targeted to specific genomic loci with guide molecules, or with sequence specifications programmed into the nuclease itself. Gene editing has been carried out with a variety of recognized methods. Widely used editing systems include CRISPR/Cas9, ZFNs, TALENs, and meganucleases. Each of these systems operate by targeting an engineered nuclease to an exact location within the genome where they bind and create sequence specific DSBs. A target DNA sequence can be deleted, modified or replaced using the cell's endogenous repair machinery. Insertions and deletions at the edit site can range in size from a large sequence to a single base pair. Nuclease engineering, optimized delivery conditions and cellular repair mechanisms enable researchers to manipulate segments of DNA and the genes they encode for.

Editing associated errors occur. In order to realize the clinical potential of gene editing treatments, all editing associated errors must be identified and quantified. Editing-associated errors can be broadly classified into three categories: mis-edits, mis-repairs, and mis-edit/mis-repair combinations. Mis-edits occur when the editing enzyme creates off-target DSBs at homologous or random sites in the genome. Mis-edits typically result in small insertions or deletions (indels) of nucleotides at unintended genomic loci.

Disclosed directional Genomic Hybridization (dGH) methods provide an efficient and practical technique for measuring on- and off-target editing events. By measuring structural variation in many single cells, disclosed methods can be used to quantitate individual on- and off-target variants, including those that are present in less than one percent of the edited cells.

In certain embodiments, a dGH probe in illustrative embodiments a set of multi-colored dGH probes can be designed to directly visualize and characterize rearrangements at edit sites. Typically, this is accomplished by targeting such dGH probe or in illustrative embodiments such set of multicolored dGH probes at a target site for gene editing. For example, 2, 3, 4, 5, 6, 7, or 8 bands generated using the set of multi-colored dGH probes can be used, wherein at least 2 of such bands flank a target edit site.

If a specific site on the genome is to be edited, a set of custom single stranded oligonucleotides to that specific site on the genome, for example a pool of oligonucleotides that make up a dGH probe or a set of dGH probes, can be developed so that the specific site on the genome can be detected and analyzed by dGH methods. For example, if a wild-type gene is to be introduced for gene therapy, then a dGH probe or in illustrative embodiments a set of multi-colored dGH probes having single stranded oligonucleotides that bind one target DNA sequence, for embodiments that utilize a single dGH probe, or two or more target DNA sequences, that span and/or flank the target edit site can be used. Another example is if a chimeric antigen receptor containing T cell or gene-edited cell is designed, then a dGH probe or in illustrative embodiments a set of multi-colored dGH probes herein can be used to identify sites of insertion

In certain aspects, a plurality of sets of dGH probes or in illustrative embodiments of multi-colored dGH probes made up of single stranded oligonucleotides whose complementary sequences are tiled across a target DNA sequence spanning an entire chromosome and covering all chromosomes to produce a multi-colored banding pattern, allow direct visualization of structural rearrangements anywhere in the genome, making it possible to discover previously unseen or unsuspected rearrangements without knowing where to look in the first place. Such methods provide a discovery tool in patients with undiagnosed diseases to detect chromosomal structural variants. Disclosed methods led to detection of a previously unknown inversion in both an undiagnosed disease patient and one of their family members.

In some embodiments, a probe having a pool of 10 to 10,000 single stranded oligonucleotides labeled with one fluorescent label (for example, blue color) is designed to target a specific gene-edited sequence or CAR-T sequence, whereas the rest of the single stranded sister chromatid is detected with a probe having single stranded oligonucleotides labeled with a different colored fluorescent label (for example, red), so that a localized dGH and a generalized whole-chromosome screen analysis can be performed and analyzed simultaneously.

### Further Banded Fluorescence Embodiments

In one illustrative embodiment, fluorescent patterns are analyzed that are generated by two or more dGH probes on a single-stranded sister chromatid. Methods are known in the art for detecting fluorescent labels on fluorescently-labeled probes. In some embodiments, methods herein include using one or more dGH probes with the same fluorescent label to label a chromatid, which can be referred to as a monochrome dGH paint. In illustrative embodiments, with non-limiting exemplary reference to Table 1, the pools of single-stranded oligonucleotides that make up different dGH probes can be labeled with different fluorescent labels that result in bands on a single-stranded sister chromatid that are of different colors (e.g., blue, green, red, magenta, yellow, orange, etc.) which, in some embodiments herein, is referred to as banded dGH. In some embodiments herein, banded dGH is also referred to herein, as multi-color dGH paint, or dGH paint with multi-color bands, especially when such methods involve larger sections or all of a chromosome or chromatid (e.g. at least 25% or 50% of a chromosome or chromatid, or an entire arm). A wide variety of fluorophores are commercially available for use as fluorescent labels to label oligonucleotides. These fluorophores absorb and emit light at a wide variety of wavelengths and can be selected for labeling the oligonucleotide of various dGH probes, such that the single-stranded sister chromatids are specifically colored with one or more bands. As a non-limiting example, with reference to Table 1, 27390 single-stranded oligonucleotides directed to the p arm of chromosome 2 are labeled with a red fluorophore, so as to generate a red band from base pairs 14497 to 9199710. This red band is observable via fluorescence microscopy, as described elsewhere herein. In some embodiments, all the single-stranded oligonucleotides from one set of dGH probes that bind target DNA segments on the same sister chromatid are fluorescently labeled with a single color, so as to paint substantially the entire, or the entire sister chromatid that single color (monochrome dGH paint). However, in illustrative embodiments, a set of two or more dGH probes each having one color/label that is different than at least one other dGH probe of the set, binding to target DNA sequences on the same chromosome/chromatid/single-stranded sister chromatid to produce a multi-colored banding pattern. Typically for such patterns, adjacent or consecutive bands are formed by adjacent or consecutive target DNA sequences that are bound by dGH probes having labels of different colors.

Multi-color dGH banding analysis can achieve both detection of bands down to a 5 Kb, 2 Kb, or 1 Kb target DNA sequence as well as globalized visualization of genome-wide information. This increased resolution can achieve identification of small structural variations, and repair events in a localized area of a chromosome or single-stranded sister chromatid. In some embodiments, such dGH banding analysis is combed with techniques disclosed herein, such as, for example, staining or monochrome dGH painting,

In certain embodiments, spectral analysis of dGH bands can achieve high resolution information of a chromosome down to fractions of a band. For example, individual dGH bands can be subdivided to the North end of a band, with data layers (e.g. oligo density differences between bands or regions of bands and/or repetitive sequence markers) providing information to a finer point. Analysis of this information can be used to determine, localize, and/or map a region of a chromosome in which a repair event, or a structural feature such as a chromosomal structural variant occurred. In certain illustrative embodiments, using dGH probes designed to detect a 1 Mb band, dGH banding can locate structural information within 500 Kb, 400 Kb, 300 Kb, 200 Kb, or 100 Kb of a breakpoint, compared to 100 Mb when using painting techniques alone. Methods provided herein using a level of condensation and even selecting or generating less condensed chromosomes or single-stranded chromatids can improve such resolution with respect to a site or region of a repair event or structural feature.

In certain embodiments, methods disclosed herein can be used to identify deletions within a band. For example, a 1 Mb deletion on chromosome 1 would not be identified with single color painting techniques, but with multi-color dGH banding methods as disclosed herein, the missing chromosome segment can be identified as an omitted band or one-half of 2 bands when compared to a banded reference chromosome.

In certain embodiments, methods disclosed herein can be used to identify localized information of complex structural variants, such as a structural variation within a structural variation. In some embodiments, dGH banding can be used to identify a deletion within an inversion. For example, if an inversion is covered by 5 bands, and one band is missing, comparison with the banding number of and pattern of a reference chromosome will identify the deleted band within the inversion.

In certain embodiments, methods disclosed herein can be used to identify small copy number variations, such as an insertion of a band. For example, an insert resulting in an additional band of 1 Mb can be visualized using dGH banding, which would not be possible using single-color fluorescence techniques.

In some embodiments, dGH banding can be used to identify viral inserts (integration sites) as small as 5 Kb, 4 Kb, 3 Kb, 2 Kb, or smaller in size, such as, 1 Kb, or 0.5 Kb. In some embodiments, dGH banding can be used to identify and differentiate two sequential inserts as small as 5 Kb, 4 Kb, 3 Kb, 2 Kb, or 1 Kb each in size.

In certain embodiments, the bands created by a set of dGH probes can provide structural resolution of a chromosome that range in size from about 150 Mb to 1 Kb. In illustrative embodiments, a set of dGH probes can provide a structural resolution of a chromosome that range in size between 20 Mb and 1 Kb. In some embodiments, a set of dGH probes can provide a structural resolution of a chromosome that range in size below 20 Mb, 15 Mb, 10 Mb, 7.5 Mb, 5 Mb, 1 Mb, 750 Kb, 500 Kb, 250 Kb, 100 Kb, 75 Kb, 50 Kb, 25 Kb, 10 Kb, 5 Kb, 4 Kb, 3 Kb, 2 Kb, or 1 Kb. In some illustrative embodiments, a set of dGH probes can provide a structural resolution of a chromosome that is of 1 Kb in size.

In some embodiments, the aspect of structural resolution of a chromosome provided by a set of dGH probes can also be interpreted in terms of size of bands that are formed on a chromosome by the set of dGH probes. Likewise, in certain embodiments, dGH probes can be designed to provide bands on a chromosome or a portion thereof, that range in size from 50 Kb to 2 Kb. In some embodiments, bands formed by dGH probes can range in size for example, between 50 Mb and 1 Kb, 30 Mb and 1 Kb, 10 Mb and 1 Kb, 1 Mb and 1 Kb, 100 Kb and 1 Kb, 50 Kb and 1 kb, 10 Kb and 2 Kb, 8 Kb and 2 Kb, 6 Kb and 2 Kb, or 4 Kb and 2 Kb.

In certain embodiments, a set of labeled dGH probes can be designed to target loci within a genome which are known to influence or cause a disease state. In one embodiment, a dGH probe set can be designed to target genes known to be associated with the development or presence of lung cancer. Similarly, a dGH probe set can be designed and utilized with the methods disclosed herein for any disease or condition of interest.

In certain embodiments, methods disclosed herein can achieve a resolution down to 10 Kb, 5 Kb, 2 Kb or 1 Kb. Thus, dGH probes can be designed to bind to target DNA sequences, and typically are complementary to a portion of a target DNA sequence, wherein the target DNA sequences have the same size as the ranges provided herein for bands.

In certain embodiments, a set of labeled dGH probes can be designed to target loci within a genome which are known to be correlated with different states of a particular disease. In one embodiment, a dGH probe set can be designed to indicate the state of disease progression, for instance in a neurodegenerative disease.

In certain embodiments, a set of labeled dGH probes can be designed to target loci within a genome which are known to be correlated with genetic disorders. In one embodiment, a dGH probe set can be designed as a prenatal diagnostic tool for genetic disorders.

In certain embodiments, a set of labeled dGH probes can be designed to target loci within a genome to provide diagnostic tools for any disease or health condition of interest. In certain embodiments, the disease or condition may be selected from diseases of the respiratory tract, musculoskeletal disorders, neurological disorders, diseases of the skin, diseases of the gastrointestinal tract and various types of cancers.

In certain embodiments, a set of labeled dGH probes can be designed to target specific classes of genes within a genome. In one embodiment, a dGH probe set can be designed to target genes for different types of kinases.

In certain embodiments, a set of labeled dGH probes can be designed to focus on research areas of interest. In one embodiment, a dGH probe set can be designed to test almost any hypotheses relating to genomic DNA sequences in the biomedical sciences.

### Kits

In some aspects, provided herein are kits that comprise two, three, four, five, six or more of the components provided for performing methods herein. Such components can include any of the components used in any of the methods disclosed herein. In some embodiments, such components include components used for dGH processing, such as used in dGH harvesting, as provided herein. As non-limiting examples, such components can include dGH probes, including dGH paints, or dGH probe sets provided herein; buffer(s); enzyme(s) (such as for degrading chromatids that include nucleoside analogs); nucleotide analogs (e.g. BrdU/C); colcemid; a fixative solution; a DNA stain (e.g. a Hoechst stain); solid support matrices optionally with one or more mask, a flow cytometry reagent, and/or a support matrix having a two-dimensional, regularly spaced arrangement of spots/positions, probes (in illustrative embodiments dGH probes or dGH probe sets), cells, and/or chromosomes. Such regularly spaced arrangement of probes can be in dry format.

Such kit components can be contained within one or more containers that can be ordered and/or shipped together, and can be included together on a virtual product area of a store, such as an online store.

### EXEMPLARY EMBODIMENTS

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional embodiments and aspects are provided in other sections herein. Furthermore, it will be understood that embodiments are provided that are specific embodiments for many aspects and that can be combined with any other embodiment, for example as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are sometimes provided as non-limiting exemplary combinations and/or are discussed more specifically in other sections of this detailed description.

Provided herein in one aspect is method for analyzing cells in a cell population, comprising
a) sorting cells in the cell population using a fluorescence-based cell sorting method, to increase the proportion of cells in metaphase, thereby providing a metaphase-enriched cell population;
b) contacting one or both of a pair of single-stranded sister chromatids from individual cells of the metaphase-enriched cell population, with a first dGH probe, the first dGH probe comprising a first colored fluorescent label of a set of fluorescent labels, wherein the first dGH probe comprises a pool of single-stranded oligonucleotides that comprise a same fluorescent label of the set of fluorescent labels, wherein each single stranded oligonucleotide of a pool binds a different complementary DNA sequence within a same target DNA sequence found on one of the single-stranded sister chromatids, and wherein the single-stranded sister chromatids are prepared by degrading a strand of a sister chromatid; and
c) detecting the first colored fluorescent label, to detect at least one structural feature if present on a chromosome of the individual cells, thereby analyzing cells in the cell population.

In another aspect is a method for detecting at least one structural feature in a chromosome of individual cells of a cell population, the method comprising the steps of:
a) applying a fluorescence-based cell sorting method to the cell population to generate a sorted subpopulation of cells, wherein the cell sorting is based on a cell cycle stage, the presence of one or more target cell surface markers, the presence of one of one or more specific chromosomes, the presence of a target DNA sequence or a set thereof, or the presence of a structural feature on the chromosome
b) contacting a pair of single-stranded sister chromatids in a metaphase spread prepared from individual cells of the metaphase-enriched cell population, with a first dGH probe, wherein each single-stranded sister chromatid is prepared by degrading a chromosome strand, wherein the first dGH probe comprising a first colored fluorescent label of a set of fluorescent labels, wherein the first dGH probe comprises a pool of single-stranded oligonucleotides that comprise a same fluorescent label of the set of fluorescent labels, and wherein each single stranded oligonucleotide of a pool binds a different complementary DNA sequence within a same target DNA sequence found on one of the single-stranded sister chromatids;
c) generating a spectral profile from one or both single-stranded sister chromatids using fluorescence detection, wherein the spectral profile is based on a hybridization pattern of the first dGH probe to one or both single-stranded sister chromatids of the pair; and
d) detecting based on the spectral profile, the presence of the at least one structural feature if present in on the chromosome from individual cells.

In another aspect herein is provided a method for detecting a structural feature in a chromosome of cells in a cell population in a two-dimensional spatial arrangement, comprising the steps of:
a) placing individual cells from the cell population in a two-dimensional, regularly spaced arrangement on a support matrix, wherein the cell population is a metaphase-enriched cell population;
b) generating a pair of single-stranded sister chromatids from a chromosome for each of the cells by degrading a strand from sister chromatids, wherein at least one of the sister chromatids comprises a target DNA sequence;
c) contacting one or both of a pair of single-stranded sister chromatids in individual cells of the cell population, with a first directional genomic hybridization (dGH), wherein the first dGH probe comprises a pool of single-stranded oligonucleotides complementary to a portion of a first target DNA sequence on one of the single-stranded sister chromatids and comprising a first colored fluorescent label; and
d) performing fluorescence analysis of one or both single-stranded sister chromatids by detecting a spectral profile generated based on a hybridization pattern of the first dGH probe to one of the single-stranded sister chromatids of the pair;
e) thereby detecting the structural feature if present on the chromosome.

In another aspect provided herein is a method for a two-dimensional spatial arrangement of chromosomes from a cell population and detection of at least one structural feature in the chromosomes, comprising the steps of:
a) placing the chromosomes into a two-dimensional, regularly spaced arrangement on a solid support;
b) contacting, on the solid support, one or both of a pair of single-stranded sister chromatids generated from each of the chromosomes, with a first directional genomic hybridization (dGH) probe, wherein each single-stranded sister chromatid is prepared by degrading a chromosome strand, and wherein each dGH probe comprises a pool of single-stranded oligonucleotides complementary to a portion of a first target DNA sequence on one of the single-stranded sister chromatids and comprising a first colored fluorescent label;
c) performing fluorescence analysis of one or both single-stranded sister chromatids by detecting a spectral profile generated based on a hybridization pattern of the first dGH probe to one of the single-stranded sister chromatids of the pair; and
d) detecting the spectral profile of one or both single-stranded sister chromatid on the solid support, thereby detecting if present, the at least one structural feature.

In some embodiments of any aspect or embodiment herein, the contacting further comprises contacting the one or both of the pair of single-stranded sister chromatids with a second dGH probe labeled with a second colored fluorescent label, wherein each single-stranded oligonucleotide of the second dGH probe is complementary to a portion of a second target DNA sequence on one of the single-stranded sister chromatids and comprises a second colored fluorescent label, and the detecting is detecting a spectral profile generated based on a hybridization pattern of the of the first dGH and the second dGH probe to one or both single stranded sister chromatids. In some embodiments of any aspects or embodiments herein, the detecting the spectral profile comprises:
d) (i) comparing the spectral profile of the one or both single-stranded sister chromatids to a reference spectral profile representing a control sequence; and
d) (ii) detecting at least one difference between the reference spectral profile and the spectral profile of the one or both single-stranded sister chromatids of the pair.

In some embodiments of any aspects or embodiments herein, the method further comprises before the contacting, placing the cells in a two-dimensional, regularly spaced arrangement on a support matrix. In some embodiments of any aspects or embodiments herein, wherein the structural feature is a structural variation. In some embodiments of any aspects or embodiments herein, wherein the method further comprises before the contacting, placing the cells in a two-dimensional, regularly spaced arrangement on a support matrix.

Provided herein in one aspect is a method for detecting a fluorescence signal in a chromosome of individual cells of a population of cells, the method comprising the steps of:
a) applying a cell sorting method to the population of cells to sort the cells based on one or more characteristics to generate a sorted cell population;
b) (performing a directional genomic hybridization (dGH) reaction by) contacting a pair of single-stranded sister chromatids in a metaphase spread prepared from individual cells of the sorted cell population, with a first dGH probe, the first dGH probe comprising a first colored fluorescent label of a set of fluorescent labels, wherein the first dGH probe comprises a pool of single-stranded oligonucleotides that comprise a same fluorescent label of the set of fluorescent labels, wherein each single stranded oligonucleotide of a pool binds a different complementary DNA sequence within a same target DNA sequence found on one of the single-stranded sister chromatids; and
c) detecting a fluorescence signal from the first colored fluorescent label, from one or both single-stranded sister chromatids. In illustrative embodiments, each single-stranded sister chromatid is prepared by degrading a chromosome strand. In illustrative embodiments, the sorting is performed using a fluorescence-based sorting method to sort cells to prepare a metaphase-enriched cell population.

In some embodiments, the one or more characteristics (e.g. one or more cell characteristics) is one or more of the presences of one or more target cell surface markers, the presence of one of one or more specific chromosomes (e.g. chromosome 8), the presence of a target DNA sequence or a set thereof, the presence of a structural feature on the chromosome, or a cell cycle stage. In some embodiments, the detecting is detecting a fluorescence pattern based on a hybridization pattern of the first dGH probe to one or both single-stranded sister chromatids of the pair. In some embodiments, the fluorescence pattern is analyzed to detect the presence of the at least one structural feature, for example a structural variation and/or repair event in the chromosome from the individual cells.

In another aspect, provided herein is a method for detecting a fluorescence signal in a chromosome of individual cells of a population of cells, the method comprising the steps of:
a) sorting the population of cells using a cell sorting method to sort the cells into a metaphase-enriched cell population;
b) contacting a pair of single-stranded sister chromatids in a metaphase spread prepared from individual cells of the metaphase-enriched cell population, with a first dGH probe, the first dGH probe comprising a first colored fluorescent label of a set of fluorescent labels, wherein the first dGH probe comprises a pool of single-stranded oligonucleotides that comprise a same fluorescent label of the set of fluorescent labels, wherein each single stranded oligonucleotide of a pool binds a different complementary DNA sequence within a same target DNA sequence found on one of the single-stranded sister chromatids, and wherein the single-stranded sister chromatids are prepared by degrading a strand of a sister chromatid; and
c) detecting the fluorescence signal from the first colored fluorescent label, from one or both single-stranded sister chromatids. In illustrative embodiments, each single-stranded sister chromatid is prepared by degrading a chromosome strand.

In one aspect, provided herein is a method for analyzing cells in a cell population, comprising
a) sorting cells in the cell population using a cell sorting method, to increase the proportion of cells in metaphase, thereby providing a metaphase-enriched cell population;
b) contacting one or both of a pair of single-stranded sister chromatids in individual cells of the metaphase-enriched cell population, with a first panel of single-stranded oligonucleotide probes that are labeled with a first colored label, wherein each probe of the first panel binds a different complementary DNA sequence within a first target DNA sequence of one chromatid of the pair of single-stranded sister chromatids to produce a spectral profile upon hybridization of the panel of oligonucleotide probes to the sister chromatids; and
c) detecting the first colored label to detect at least one structural feature of a chromosome of the individual cells, thereby analyzing cells in the cell population.

In some embodiments, the contacting further comprises contacting the one or both of the pair of single-stranded sister chromatids with a second panel of single-stranded oligonucleotide probes that are labeled with a second colored label, wherein each probe of the second panel binds a different complementary DNA sequence within a second target DNA sequence on the one chromatid of the pair of single-stranded sister chromatids, and the detecting is detecting the first colored label and the second colored label to detect the at least one structural feature.

In some embodiments, the method further comprises analyzing a spectral profile of one or both of the pair of single-stranded sister chromatids utilizing the detected first colored label and second colored label, to detect at least one structural feature of a chromosome of the individual cells, thereby analyzing cells in the cell population.

In one aspect, provided herein is a method for detecting at least one structural feature in a chromosome of individual cells of a population of cells, comprising the steps of:
a) applying a cell sorting method to increase the proportion of cells in metaphase, in the population of cells, thereby generating a metaphase-enriched cell population;
b) generating a pair of single-stranded sister chromatids from said chromosome in individual cells of the metaphase-enriched cell population, wherein each sister chromatid comprises one or more target DNA sequences;
c) contacting one or both single-stranded sister chromatid with two one or more oligonucleotide probes, in illustrative embodiments two or more panels or probes, wherein each of the oligonucleotide probes is single-stranded and complementary to at least a portion of a target DNA sequence and wherein each of the probes comprises at least one label and at least two of the probes complementary to said target DNA sequence comprise labels of different colors such that a spectral profile of one or both single-stranded sister chromatid is produced by the hybridization pattern of the at least two probes to one or both single-stranded sister chromatid;
d) detecting the spectral profile of one or both single-stranded sister chromatid;
e) comparing the spectral profile of step (c) to a reference spectral profile representing a control; and
f) detecting, based on at least one spectral difference between either or both spectral profile of step (c) and the reference spectral profile, the presence of the at least one structural feature.

In some embodiments, of any aspects or embodiments herein, the method comprises degrading one chromatid strand of the chromosome to generate the pair of single-stranded sister chromatids.

In some embodiments, of any aspects or embodiments herein, the cell sorting method is selected from the group consisting of a centrifugation-based cell sorting method, a filtration-based cell sorting method, an immobilization-based cell sorting method, a bead-based cell sorting method, and a fluorescence-based sorting method. In some embodiments, the cell sorting method is an automated cell sorting method. In some embodiments, the cell sorting method comprises labeling cells with a DNA stain. In some embodiments, the DNA stain is a fluorochrome. In some embodiments, the DNA stain is selected from the group consisting of propidium iodine, 7-AAD and Hoechst, chromomycin A3, quinacrine, daunomycin, or any other fluorochromes that bind chromosomes through DNA intercalation or that bind a secondary structure of DNA.

In some embodiments, the one or more cell of the population of cells expresses a reporter protein. In some embodiments, the cell sorting method comprises sorting cells based on cell surface expressed markers.

In one aspect, provided herein is a method for detecting a structural feature in a chromosome of cells in a two-dimensional spatial arrangement, comprising the steps of:
a) placing cells in the two-dimensional spatial arrangement of partitions;
b) generating a pair of single-stranded sister chromatids from said chromosome, wherein each sister chromatid comprises one or more target DNA sequence;
c) contacting one or both single-stranded sister chromatid with two or more oligonucleotide probes wherein each of the probes is single-stranded, unique and complementary to at least a portion of a target DNA sequence and wherein each of the probes comprises at least one label and at least two of the probes complementary to said target DNA sequence comprise labels of different colors such that a spectral profile of one or both single-stranded sister chromatid is produced by the hybridization pattern of the at least two probes to one or both single-stranded sister chromatid;
d) detecting the spectral profile of one or both single-stranded sister chromatid;
e) comparing the spectral profile of step (c) to a reference spectral profile representing a control; and
f) detecting, based on at least one spectral difference between either or both spectral profile of step (c) and the reference spectral profile, the presence of the at least one structural feature.

In one aspect provided herein is a method for two-dimensional spatial arrangement of chromosomes in partitions and detection of at least one structural feature in a chromosome, comprising the steps of:
a) placing chromosomes into one or more partition of a two-dimensional spatial arrangement of partitions;
b) generating a pair of single-stranded sister chromatids from said chromosome, wherein each sister chromatid comprises one or more target DNA sequence;
c) contacting one or both single-stranded sister chromatid with two or more oligonucleotide probes wherein each of the probes is single-stranded, unique and complementary to at least a portion of a target DNA sequence and wherein each of the probes comprises at least one label and at least two of the probes complementary to said target DNA sequence comprise labels of different colors such that a spectral profile of one or both single-stranded sister chromatid is produced by the hybridization pattern of the at least two probes to one or both single-stranded sister chromatid;
d) detecting the spectral profile of one or both single-stranded sister chromatid;
e) comparing the spectral profile of step (c) to a reference spectral profile representing a control; and
f) detecting, based on at least one spectral difference between either or both spectral profile of step (c) and the reference spectral profile, the presence of the at least one structural feature.

In some embodiments, of any aspects or embodiments herein, the method further comprises
d) comparing the spectral profile to a reference spectral profile representing a control for the one chromatid; and
detecting, based on at least one spectral difference between either or both spectral profile of
step (b) and the reference spectral profile, the presence of the at least one structural feature.

In some embodiments of the aspects herein with step b) generating and step c) contacting, the contacting can be performed before the generating.

In some embodiments of the aspects or embodiments herein, the method further comprises sorting cells using a fluorescence-based sorting method to generate the metaphase-enriched cell population.

In another aspect, provided herein is a method for detecting a target DNA sequence in a chromosome of individual cells of a population of cells, the method comprising the steps of:
a) applying a cell sorting method to the population of cells to generate a sorted subpopulation of cells, wherein the cell sorting is based on a cell cycle stage, the presence of one or more target cell surface markers, the presence of one of one or more specific chromosomes, the presence of a target DNA sequence or a set thereof, or the presence of a structural feature on the chromosome,
b) contacting a pair of single-stranded sister chromatids in a metaphase spread prepared from individual cells of the metaphase-enriched cell population, with a first dGH probe, the first dGH probe comprising a first colored fluorescent label of a set of fluorescent labels, wherein the first dGH probe comprises a pool of single-stranded oligonucleotides that comprise a same fluorescent label of the set of fluorescent labels, wherein each single stranded oligonucleotide of a pool binds a different complementary DNA sequence within a same target DNA sequence found on one of the single-stranded sister chromatids; and
c) detecting a fluorescence signal from a hybridization pattern of the first dGH probe to one or both single-stranded sister chromatids of the pair, thereby detecting the target DNA sequence in the chromosome.

In some embodiments, detecting the target DNA sequence, or a plurality of such target DNA sequences, can be used to detect a structure feature such as, for example, a structural variation, and/or to detect a repair event in the chromosome.

Accordingly, in another aspect, provided herein is a method for detecting at least one structural feature such as a structural variation and/or a repair event in a chromosome of individual cells of a population of cells, the method comprising the steps of:
a) applying a cell sorting method to the population of cells to generate a sorted subpopulation of cells, wherein the cell sorting is based on a cell cycle stage, the presence of one or more target cell surface markers, the presence of one of one or more specific chromosomes, the presence of a target DNA sequence or a set thereof, or the presence of a structural feature on the chromosome,
b) contacting a pair of single-stranded sister chromatids in a metaphase spread prepared from individual cells of the metaphase-enriched cell population, with a first dGH probe, the first dGH probe comprising a first colored fluorescent label of a set of fluorescent labels, wherein the first dGH probe comprises a pool of single-stranded oligonucleotides that comprise a same fluorescent label of the set of fluorescent labels, wherein each single stranded oligonucleotide of a pool binds a different complementary DNA sequence within a same target DNA sequence found on one of the single-stranded sister chromatids;
c) generating a fluorescence pattern from one or both single-stranded sister chromatids using fluorescence detection, wherein the fluorescence pattern is based on a hybridization pattern of the first dGH probe to one or both single-stranded sister chromatids of the pair; and
d) detecting based on the fluorescence pattern, the presence of the at least one structural variation and/or repair event in the chromosome from individual cells. The contacting step is typically performing a directional genomic hybridization (dGH) reaction.

In another aspect provided herein is a method for detecting a target DNA sequence on a target chromosome from a population of cells, comprising:
a) hybridizing a set of single-stranded sister chromatids comprising a chromatid derived from the target chromosome with a preliminary directional genomic hybridization (dGH) probe, wherein the preliminary dGH probe comprises a pool of single-stranded oligonucleotides complementary to a portion of a first target DNA sequence on one of the single-stranded sister chromatids or the single-stranded chromosomes, and comprising a preliminary fluorescent label; staining the single-stranded chromatids or the chromosomes obtained after the hybridizing of step (a) with a DNA stain to obtain a stained chromatid suspension or a stained chromosome suspension;
b) applying a fluorescence sorting method on the stained chromatid suspension or the stained chromosome suspension to obtain an enriched chromatid suspension comprising a chromatid derived from the target chromosome or an enriched chromosome suspension comprising the target chromosome;
c) placing the chromatids from the enriched chromatid suspension into one or more partition of a two-dimensional spatial arrangement of partitions, or onto the same addressable position within each partition of a set of partitions; subjecting each of the chromatids present in one or more partitions or the same addressable position to a fluorescence-based detection method comprising a first dGH probe comprising a first colored fluorescent label; and
d) performing fluorescence analysis of the chromatids present in one or more partitions or same addressable position, by detecting fluorescence signals generated based on a hybridization pattern of the first dGH probe to the single-stranded chromatids comprising a chromatid derived from the target chromosome to detect the target DNA sequence. In some embodiments, the detecting detects a structural feature in at least one of the chromatids in at least one of the partitions or the same addressable position.

In another aspect, provided herein is a method for detecting a structural feature or repair event on a target chromosome from a population of cells, comprising:
a. hybridizing a set of single-stranded sister chromatids comprising a chromatid derived from the target chromosome with a preliminary directional genomic hybridization (dGH) probe, wherein the preliminary dGH probe comprises a pool of single-stranded oligonucleotides complementary to a portion of a first target DNA sequence on one of the single-stranded sister chromatids, and comprising a preliminary fluorescent label;
b. optionally staining the single-stranded chromatids obtained after the hybridizing of step (a) with a DNA stain to obtain a stained chromatid suspension;
c. applying a fluorescence sorting method on the stained chromatid suspension to obtain an enriched chromatid suspension comprising a chromatid derived from the target chromosome;
d. placing the chromatids from the enriched chromatid suspension into one or more partition of a two-dimensional spatial arrangement of partitions, or onto the same addressable position within each partition of a set of partitions;
e. subjecting each of the chromatids present in one or more partitions to a fluorescence-based detection method comprising a first dGH probe comprising a first colored fluorescent label; and
f. performing fluorescence analysis of the chromatids comprising chromatid derived from the target chromosome present in one or more partitions, by detecting fluorescence signals generated based on a hybridization pattern of the first dGH probe to the single-stranded chromatids comprising a chromatid derived from the target chromosome to detect the structural feature in each of the chromatids or chromosomes in each of the partitions. In some embodiments of the above aspect, the process prior to step (a) comprises: incubating the population of cells in the presence of a DNA analog for one cell cycle, and generating a set of single-stranded sister chromatids comprising a chromatid derived from the target chromosome from at least one cell of a metaphase-enriched cell population of the population of cells,
g. (a) hybridizing the set of single-stranded sister chromatids comprising a chromatid derived from the target chromosome,
h. (b) staining the single-stranded chromatids to obtain a stained chromatid suspension,
i. (c) applying a fluorescence sorting method on the stained chromatid suspension,
j. (d) placing the chromatids from the enriched chromatid suspension into one or more partition of a two-dimensional spatial arrangement of partitions,
k. (e) subjecting each of the chromatids placed into one or more partitions to a fluorescence-based detection method comprising:
l. contacting one or both of the pair of single-stranded sister chromatids, with the first dGH probe, wherein the first dGH probe comprises a pool of single-stranded oligonucleotides complementary to a portion of a first target DNA sequence on one of the single-stranded sister chromatids and comprising a first colored fluorescent label, and
m. performing the fluorescence analysis of one or both single-stranded sister chromatids by detecting fluorescence signals generated based on a hybridization pattern of the first dGH probe to one of the single-stranded sister chromatids of the pair.

In some embodiments, of any of the aspects or embodiments herein, the contacting further comprises contacting the one or both of the pair of single-stranded sister chromatids with a second dGH probe labeled with a second colored fluorescent label, wherein each single-stranded oligonucleotide of the second dGH probe is complementary to a portion of a second target DNA sequence on one of the single-stranded sister chromatids and comprises a second colored fluorescent label, and the detecting is detecting the first colored fluorescent label and the second colored fluorescent label thereby detecting the structural feature in each of the chromosomes in each of the partitions.

In some embodiments, of any of the aspects or embodiments herein, comprising the steps for detecting a structural feature or repair event on a target chromosome from a population of cells, the method comprises:
a) hybridizing a set of single-stranded chromosomes comprising the target chromosome, with a preliminary directional genomic hybridization (dGH) probe, wherein the preliminary dGH probe comprises a pool of single-stranded oligonucleotides complementary to a portion of a first target DNA sequence on the single-stranded chromosomes, and comprising a preliminary fluorescent label;
b) staining the chromosomes obtained after the hybridizing of step (a) with a DNA stain to obtain a stained chromosome suspension;
c) applying a fluorescence sorting method on the stained chromosome suspension to obtain an enriched chromosome suspension comprising the target chromosome;
d) placing the chromosomes from the enriched chromosome suspension or into one or more partition of a two-dimensional spatial arrangement of partitions;
e) subjecting each of the chromosomes placed into one or more partitions to a fluorescence-based detection method, wherein the fluorescence-based detection method comprises:
   i. generating denatured chromosomes comprising single stranded chromosomes from each of the chromosomes placed into one or more partitions, wherein at least one of the single-stranded chromosomes comprises a target DNA sequence of the target chromosome; and
   ii. contacting the single stranded chromosomes with the first dGH probe, wherein the first dGH probe comprises a pool of single-stranded oligonucleotides complementary to a portion of a first target DNA sequence on one or more of the single-stranded denatured chromosomes and comprising a first colored fluorescent label; and
f) performing fluorescence analysis of the chromosomes comprising the target chromosome placed into one or more partitions, by detecting fluorescence signals generated based on a hybridization pattern of the first dGH probe to the single-stranded chromosomes comprising the target chromosome.

In some embodiments of any of the aspects or embodiments herein comprising the steps for detecting a structural feature or repair event on a target chromosome from a population of cells, the process prior to step (a) comprises: incubating the population of cells in the presence of a DNA analog for one cell cycle,
(e) subjecting each of the chromatids or the chromosomes placed into one or more partitions to a fluorescence-based detection method comprises:
   (i) generating a set of single-stranded sister chromatids comprising a chromatid derived from the target chromosome from at least one cell of a metaphase-enriched cell population of the population of cells, and
   (ii) contacting one or both of the pair of single-stranded sister chromatids, with the first dGH probe, wherein the first dGH probe comprises a pool of single-stranded oligonucleotides complementary to a portion of a first target DNA sequence on one of the single-stranded sister chromatids and comprising a first colored fluorescent label, and
(f) performing the fluorescence analysis of one or both single-stranded sister chromatids by detecting fluorescence signals generated based on a hybridization pattern of the first dGH probe to one of the single-stranded sister chromatids of the pair.

In another aspect, provided herein method for detecting one or more target chromosomes comprising a structural feature from a population of cells, said method comprising:
a) generating a pair of single-stranded sister chromatids from each of the chromosomes isolated from the population of cells, wherein at least one of the sister chromatids is derived from the target chromosome and comprises a target DNA sequence; and
b) contacting one or both of the pair of single-stranded sister chromatids, with a first dGH probe, wherein the first dGH probe comprises a pool of single-stranded oligonucleotides complementary to a portion of a first target DNA sequence on one of the single-stranded sister chromatids and comprising a first colored fluorescent label;
c) optionally staining the single-stranded sister chromatids obtained after step (b) with a DNA stain to obtain a stained chromatid suspension;
d) applying a fluorescence sorting method to obtain an enriched chromatid suspension; and
e) performing fluorescence analysis of one or both single-stranded sister chromatids by detecting fluorescence signals generated based on a hybridization pattern of the first dGH probe to one of the single-stranded sister chromatids of the pair, thereby detecting the one or more target chromosomes comprising the structural feature from the population of cells.

In some embodiments, of any aspects or embodiments herein, the method further comprises before the step of a) generating a pair of single-stranded sister chromatids from each of the chromosomes isolated from the population of cells, wherein at least one of the sister chromatids is derived from the target chromosome and comprises a target DNA sequence, contacting cells of the population of cells with a first directional genomic hybridization (dGH) probe under permeabilizing conditions such that the first dGH probe enters the nucleus of the cells, and wherein the hybridizing occurs within the nucleus of the cells.

In some embodiments of any aspects or embodiments herein, the method further comprises enumerating, for example by counting the number of chromosomes with the structural feature or by generating a numbered list of structural features identified on the target chromosomes the structural features on the target chromosome from the population of cells. In some embodiments, the method is useful for determining the number, percentage or ratio of chromosomes of the chromosomes isolated from the population of cells, that are a target chromosome having the structural feature. In some embodiments, the method further comprises enumerating the structural features on the target chromosome from the population of cells. In some embodiments of any aspects or embodiments herein, the structural feature comprises a structural variation on the target chromosome.

In some embodiments, of any aspects or embodiments herein, the contacting further comprises contacting the one or both of the pair of single-stranded sister chromatids with a second dGH probe labeled with a second colored fluorescent label, wherein each single-stranded oligonucleotide of the second dGH probe is complementary to a portion of a second target DNA sequence on one of the single-stranded sister chromatids and comprises a second colored fluorescent label, and the detecting is detecting the first colored fluorescent label and the second colored fluorescent label thereby detecting the structural feature on the target chromosomes from the population of cells.

In another aspect, provided herein is a method for detecting at least one structural feature such as a structural variation and/or repair event in in a chromosome of individual cells of a population of cells, the method comprising the steps of:
a) applying a cell sorting method to increase the proportion of cells in metaphase, in the population of cells, thereby generating a metaphase-enriched cell population;
b) performing a directional genomic hybridization (dGH) reaction by contacting a chromosome or a pair of single-stranded sister chromatids in a metaphase spread prepared from individual cells of the metaphase-enriched cell population, with a first dGH probe, the first dGH probe comprising a fluorescent label of a set of fluorescent labels, wherein the first dGH probe comprises a pool of single-stranded oligonucleotides that comprise a same fluorescent label of the set of fluorescent labels, wherein each single stranded oligonucleotide of a pool binds a different complementary DNA sequence within a same target DNA sequence found on one of the single-stranded sister chromatids;
c) generating a fluorescence pattern from the chromosome or one or both single-stranded sister chromatids using fluorescence detection, wherein the fluorescence pattern is based on a hybridization pattern of the first dGH probe to the chromosome or the one or both single-stranded sister chromatids of the pair; and
d) detecting based on the fluorescence pattern, the presence of the at least one structural variation and/or repair event in the chromosome from individual cells.

In one aspect, provided herein is a method for detecting a structural feature in a chromosome of cells in a two-dimensional spatial arrangement, comprising the steps of:
a) placing the cells in the two-dimensional spatial arrangement of partitions;
b) generating a pair of single-stranded sister chromatids from a chromosome for each the cells, wherein at least one of the sister chromatids comprises two or more target DNA sequences;
c) contacting one or both of a pair of single-stranded sister chromatids in individual cells of the metaphase-enriched cell population, with a first directional genomic hybridization (dGH) probe in a metaphase spread generated from a cell of the metaphase-enriched cell population, wherein each dGH probe comprises a pool of single-stranded oligonucleotides complementary to a portion of a first target DNA sequence on one of the single-stranded sister chromatids and comprising a first colored fluorescent label;
d) performing fluorescence analysis of one or both single-stranded sister chromatids by detecting fluorescence signals generated based on a hybridization pattern of the first dGH probe to one of the single-stranded sister chromatids of the pair; and
e) detecting the first colored fluorescent label to detect at least one structural feature and/or repair event of a chromosome for each of the individual cells, thereby analyzing cells in the cell population.

In one aspect, provided herein is a method for analyzing cells in a cell population, comprising the steps of:
a) sorting cells in the cell population using a cell sorting method, to increase the proportion of cells in metaphase, thereby providing a metaphase-enriched cell population;
b) generating a pair of single-stranded sister chromatids from at least one cell of the metaphase-enriched cell population, wherein at least one of the sister chromatids comprises two or more target DNA sequences;
c) contacting one or both of the pair of single-stranded sister chromatids, with a first directional genomic hybridization (dGH) probe in a metaphase spread generated from a cell of the metaphase-enriched cell population, wherein each dGH probe comprises a pool of single-stranded oligonucleotides complementary to a portion of a first target DNA sequence on one of the single-stranded sister chromatids and comprising a first colored fluorescent label;
d) performing fluorescence analysis of one or both single-stranded sister chromatids by detecting fluorescence signals generated based on a hybridization pattern of the first dGH probe to one of the single-stranded sister chromatids of the pair; and
e) detecting the first colored fluorescent label to detect at least one structural feature and/or repair event of a chromosome of the individual cells, thereby analyzing cells in the cell population.

In some embodiments, of any of the aspects or embodiments herein, prior to contacting, single-stranded sister chromatids are prepared from the metaphase-enriched cell population by degrading a newly synthesized chromosome strand. In some embodiments, the degrading is performed by incorporating a DNA analog into the individual cells of the population of cells for once cycle, and stripping the newly synthesized chromosome strand that incorporates the DNA analog. In some embodiments, of any aspects or embodiment herein, prior to contacting, single-stranded chromatids are prepared by denaturing the chromosomes from the metaphase-enriched cell population. In some embodiments, the denaturing is a temperature-induced denaturation.

In some embodiments of any of the aspects or embodiments herein, the step of contacting further comprises contacting the one or both of the pair of single-stranded sister chromatids with a second dGH probe labeled with a second colored fluorescent label, wherein each single-stranded oligonucleotide of the second dGH probe is complementary to a portion of a second target DNA sequence on one of the single-stranded sister chromatids and comprises a second colored fluorescent label, and the detecting is detecting the first colored fluorescent label and the second colored fluorescent label thereby detecting the at least one structural feature and/or repair event.

In some embodiments, the method further comprises analyzing a spectral profile of one or both of the pair of single-stranded sister chromatids utilizing the detected first colored fluorescent label and second colored fluorescent label, to detect at least one structural feature and/or repair event of one or more chromosomes. In some embodiments, the detecting comprises detecting at least one structural feature and/or repair event a chromosome of the individual cells, thereby analyzing cells in the cell population.

In some embodiments, the method further comprises comparing the fluorescence analysis with reference fluorescence information representing a control sequence.

In one aspect, provided herein is a method for two-dimensional spatial arrangement of chromosomes in partitions and detection of at least one structural feature in chromosomes, comprising the steps of:
a) placing the chromosomes into one or more partition of a two-dimensional spatial arrangement of partitions;
b) contacting within each of the partitions, one or both of a pair of single-stranded sister chromatids generated from each of the chromosomes, with a first directional genomic hybridization (dGH) probe in a metaphase spread, wherein each dGH probe comprises a pool of single-stranded oligonucleotides complementary to a portion of a first target DNA sequence on one of the single-stranded sister chromatids and comprising a first colored fluorescent label;
c) performing fluorescence analysis of one or both single-stranded sister chromatids by detecting fluorescence signals generated based on a hybridization pattern of the first dGH probe to one of the single-stranded sister chromatids of the pair; and
d) detecting the first colored fluorescent label to detect at least one structural feature and/or repair event of each of the chromosomes in each of the partitions.

In one aspect, provided herein is a method for generating a multi-color fluorescence pattern on a single-stranded sister chromatid of a pair of single-stranded sister chromatids, comprising the steps of: (a) generating the pair of single-stranded sister chromatids from a chromosome; (b) contacting one or both single-stranded sister chromatids with two or more directional genomic hybridization (dGH) probes each comprising a fluorescent label from a set of at least two fluorescent labels capable of emitting different colors; (c) performing fluorescence analysis of one or both single-stranded sister chromatids of the pair by detecting fluorescence signals generated based on a hybridization pattern of the two or more dGH probes to the single-stranded sister chromatid; and (d) generating, based on the fluorescence analysis, the multi-color fluorescence pattern on the single-stranded sister chromatid. In illustrative embodiments, the multi-color fluorescence pattern comprises bands having the different colors of the at least two fluorescent labels. In illustrative embodiments, the multi-color fluorescent pattern is used to detect and/or classify at least one structural feature, such as a structural variant or to detect a chromosome repair event.

In one aspect, provided herein is a method for generating a multi-color fluorescence pattern on a chromosome, comprising the steps of: (a) generating a denatured chromosome; (b) contacting the denatured chromosome with two or more directional genomic hybridization (dGH) probes each comprising a fluorescent label from a set of at least two fluorescent labels capable of emitting different colors; (c) performing fluorescence analysis of the denatured chromosome by detecting fluorescence signals generated based on a hybridization pattern of the two or more dGH probes to the denatured chromosome; and (d) generating, based on the fluorescence analysis, the multi-color fluorescence pattern on the chromosome. In illustrative embodiments, the multi-color fluorescence pattern comprises bands having the different colors of the at least two fluorescent labels. In illustrative embodiments, the multi-color fluorescent pattern is used to detect and/or classify at least one structural feature, such as a structural variant or to detect a chromosome repair event.

In some embodiments of any of the aspects or embodiments that include a method as disclosed herein, prior to the contacting, single-stranded chromatids are prepared by denaturing the chromosomes from the metaphase-enriched cell population. In some embodiments, the denaturing is a temperature-induced denaturation. In some embodiments, the temperature-induced denaturation comprises exposing the chromosomes to a temperature in the excess of 75° C., 80° C., 85° C., 90° C. or higher.

In one aspect, provided herein is a method for detecting and/or classifying at least one structural feature or repair event of a chromosome of a cell, comprising the steps of: (a) generating a pair of single-stranded sister chromatids from the chromosome, wherein at least one of the sister chromatids comprises two or more target DNA sequences; (b) contacting one or both single-stranded sister chromatids with two or more directional genomic hybridization (dGH) probes in a metaphase spread generated from the cell, wherein each dGH probe comprises a pool of single-stranded oligonucleotides complementary to at least a portion of one of the two or more target DNA sequences and comprising the same label, and wherein at least two, three, four or five of the two or more dGH probes each bind to a different one of the two or more target DNA sequences and each comprise a label of a different color; (c) performing fluorescence analysis of one or both single-stranded sister chromatids by detecting fluorescence signals generated based on a hybridization pattern of the at least two, three, four, or five dGH probes to one or both single-stranded sister chromatids of the pair; and (d) detecting, based on the fluorescence analysis, the presence of the structural feature or the repair event.

In some embodiments, the method further comprises comparing the fluorescence analysis with reference fluorescence information representing a control sequence. In some embodiments, the method is used to detect the structural feature of the chromosome and the structural feature is the presence of at least one structural variation. In some embodiments, the method is used to detect the repair event. In some embodiments, performing fluorescence analysis comprises generating spectral measurements. In some embodiments, performing fluorescence analysis comprises generating a fluorescence pattern from one or both single-stranded sister chromatids. In some embodiments, the structural feature of the chromosome is the presence of at least one structural variation and/or repair event. In some embodiments, the structural feature is a structural variation. In some embodiments, of any of the aspects or embodiments herein, the feature is a wild-type genetic feature or a genetic feature referenced as normal.

In some embodiments, of any aspects or embodiments herein, the method further comprises before the contacting, placing the cells in a two-dimensional spatial arrangement of partitions and/or wherein the cells are derived from the same cell sample. In some embodiments, the method further comprises before the contacting, placing the cells in a two-dimensional spatial arrangement of partitions and/or wherein the cells are derived from more than one cell sample.

In some embodiments, of any aspects or methods herein, the two or more dGH probes are the same for each partition. In some embodiments, the two or more dGH probes in a first partition comprise at least one difference in nucleic acid sequences in comparison to the two or more oligonucleotide probes in a second partition.

In another aspect, provided herein is a method for detecting at least one structural feature in a target chromosome of individual cells of a population of cells, the method comprising the steps of:
a) applying a cell sorting method to the population of cells to generate a sorted cell population, wherein the cell sorting is based on a cell cycle stage, the presence of one or more target cell surface markers, the presence of one of one or more specific chromosomes, the presence of a target DNA sequence or a set thereof, or the presence of a structural feature on the chromosome;
b) placing the cells from the sorted cell population into one or more partition of a two-dimensional spatial arrangement of partitions, or onto the same addressable position within each partition of a set of partitions;
c) generating denatured chromatids derived from the target chromosome, comprising single stranded chromatid from each of the chromatids placed into one or more partitions, wherein at least one single-stranded comprises a target DNA sequence from the target chromosome;
d) contacting the at least one single-stranded chromatid with a first dGH probe, wherein the first dGH probe comprises a pool of single-stranded oligonucleotides complementary to a portion of a first target DNA sequence on one or more of the single-stranded chromatid and comprising a first colored fluorescent label; and
e) performing fluorescence analysis of the chromatids placed into one or more partitions, by detecting fluorescence signals generated based on a hybridization pattern of the first dGH probe to the at least one single-stranded chromatid, thereby detecting the at least one structural feature.

In one aspect, provided herein is a method for detecting at least one structural feature in a target chromosome of individual cells of a population of cells, the method comprising the steps of:
a) applying a cell sorting method to the population of cells for to increase the proportion of cells in metaphase, in the population of cells, thereby to generating generate a metaphase-enriched cell sorted population of cells, wherein the cell sorting is based on a cell cycle stage, the presence of one or more target cell surface markers, the presence of one of one or more specific chromosomes, the presence of a target DNA sequence or a set thereof, the presence of a structural feature on the chromosome, or a cell cycle stage;
a) applying a cell sorting method to the population of cells to generate a sorted cell population, wherein the cell sorting is based on a cell cycle stage, the presence of one or more target cell surface markers, the presence of one of one or more specific chromosomes, the presence of a target DNA sequence or a set thereof, or the presence of a structural feature on the chromosome;
b) placing the cells from the sorted cell population into one or more partition of a two-dimensional spatial arrangement of partitions, or onto the same addressable position within each partition of a set of partitions;
c) generating denatured chromatids derived from the target chromosome, comprising single stranded chromatid from each of the chromatids placed into one or more partitions, wherein at least one single-stranded comprises a target DNA sequence from the target chromosome; and
d) contacting the at least one single-stranded chromatid with a first dGH probe, wherein the first dGH probe comprises a pool of single-stranded oligonucleotides complementary to a portion of a first target DNA sequence on one or more of the single-stranded chromatid and comprising a first colored fluorescent label; and
(e) performing fluorescence analysis of the chromatids placed into one or more partitions, by detecting fluorescence signals generated based on a hybridization pattern of the first dGH probe to the at least one single-stranded chromatid, thereby detecting the at least one structural feature.

In some embodiments of any aspects or methods herein, the contacting comprises two or more dGH probes, each dGH probe comprising a fluorescent label of a set of fluorescent labels, wherein at least two of the two or more dGH probes each binds to a different target DNA sequence on the target chromosome and each comprises a fluorescent label of a different color. In some embodiments, generating the denatured chromosomes comprises exposing the chromosomes to a temperature that leads to denaturation of the chromosomes into single-stranded chromosomes. In some embodiments, of any aspects or methods herein, the chromosomes are exposed to a temperature in the range of 75-100° C. that leads to denaturation. In some embodiments of any aspects or embodiments herein, at least one of the sister chromatids comprises two or more target DNA sequence, and wherein the contacting comprises contacting the pair of single-stranded sister chromatids in the metaphase spread, with two or more dGH probes, wherein at least two of the two or more dGH probes each binds to a different target DNA sequence of the two or more target DNA sequences, and each comprises a fluorescent label of a different color.

In one aspect, provided herein is a method for detecting and/or classifying at least one structural feature, which in non-limiting embodiments is a structural variation and/or repair event in a chromosome from a cell, the method comprising the steps of: (a) performing a directional genomic hybridization (dGH) reaction by contacting a pair of single-stranded sister chromatids generated from the chromosome in a metaphase spread prepared from the cell, with two or more dGH probes, each dGH probe comprising a fluorescent label of a set of fluorescent labels, wherein each dGH probe comprises a pool of single-stranded oligonucleotides that comprise a same fluorescent label of the set of fluorescent labels, wherein each single stranded oligonucleotide of a pool binds a different complementary DNA sequence within a same target DNA sequence found on one of the single-stranded sister chromatids, wherein at least two of the two or more dGH probes each binds to a different target DNA sequence on one of the single-stranded sister chromatids and each comprises a fluorescent label of a different color; (b) generating a fluorescence pattern from one or both single-stranded sister chromatids using fluorescence detection, wherein the fluorescence pattern is based on a hybridization pattern of the two or more dGH probes to one or both single-stranded sister chromatids of the pair; and (c) detecting based on the fluorescence pattern, the presence of the at least one structural feature, which in non-limiting embodiments is a structural variation and/or repair event in the chromosome from the cell. In some embodiments, the detecting based on the fluorescence pattern comprises
(c) (i) comparing the fluorescence pattern of the one or both single-stranded sister chromatids to a reference fluorescence pattern representing a control sequence; and
(c) (ii) detecting at least one difference between the reference fluorescence pattern and the fluorescence pattern of the one or both single-stranded sister chromatids of the pair.

In some embodiments, the method further comprises before the contacting, placing the cells in a two-dimensional spatial arrangement of partitions and/or wherein the cell sorting method is selected from the group consisting of a centrifugation-based cell sorting method, a filtration-based cell sorting method, an immobilization-based cell sorting method, a bead-based cell sorting method, and a fluorescence-based sorting method. In some embodiments, of any of the aspects or embodiments herein, the cells have been enriched for cells in metaphase.

In some embodiments, the method comprises performing a dGH reaction and/or the cell sorting method is an automated cell sorting method. In some embodiments, the cell sorting method comprises labeling the single stranded sister chromatids, chromatids that comprise the single-stranded sister chromatids, chromosomes from the population of cells, or cells from the population with a DNA stain. In some embodiments, a method herein further comprises labeling the single-stranded sister chromatids with a DNA stain. one of the preceding claims, wherein the method further comprises labeling chromatids that comprise the single-stranded sister chromatids within the population of cells with a DNA stain. In some embodiments, the cell sorting method uses the DNA stain to generate the sorted population of cells. In some embodiments, the DNA stain is a fluorochrome. In some embodiments, the DNA stain is selected from the group consisting of propidium iodine, 7-AAD, Hoechst, chromomycin A3, quinacrine, daunomycin, or any other fluorochromes that bind chromosomes through DNA intercalation or that bind a secondary structure of DNA.

In some embodiments, the one or more cell of the population of cells expresses a reporter protein. In some embodiments, the cell sorting method comprises sorting cells based on cell surface expressed markers. In some embodiments of any aspects or embodiments herein, the method includes a step of enriching metaphase cells in the population of cells.

In one aspect, provided herein is a method for detecting and/or classifying at least one structural feature, in non-limiting embodiments a structural variation and/or repair event in a chromosome from a cell, comprising the steps of: (a) generating a pair of single-stranded sister chromatids from said chromosome, wherein at least one single-stranded sister chromatid from the pair, comprises two or more target DNA sequences; (b) after step (a) contacting one or both single-stranded sister chromatid with a stain in a metaphase spread generated from the cell; (c) after step (a) contacting one or both single-stranded sister chromatid with two or more directional genomic hybridization (dGH) probes in the metaphase spread, wherein each dGH probe comprises a pool of single stranded oligonucleotides complementary to at least a portion of one of the two or more target DNA sequences, wherein each of the two or more dGH probes comprises at least one label, wherein at least two of the two or more dGH probes each binds to a different target DNA sequence on one of the single-stranded sister chromatids, and each comprises a label of a different color; (d) detecting a staining pattern of one or both single-stranded sister chromatid, wherein the staining pattern is generated based on binding of the stain to the one or both single-stranded sister chromatid; (e) generating a fluorescence pattern for one or both single-stranded sister chromatids using fluorescence detection, wherein the fluorescence pattern is based on a hybridization pattern of the at least two dGH probes to one or both single-stranded sister chromatids of the pair; (f) comparing the staining pattern of one or both single-stranded sister chromatid of step (d) to a reference staining pattern representing a control sequence; and further comparing the fluorescence pattern of step (e) to a reference fluorescence pattern representing the control sequence; and (g) determining, based on at least one staining difference between the staining pattern of one or both single-stranded sister chromatid of step (d) and the reference staining pattern and further based on at least one difference in the fluorescence pattern for one or both single-stranded sister chromatids using fluorescence detection of step (e) and the reference fluorescence pattern, the presence of the at least one structural feature, which in non-limiting embodiments is a structural variation and/or repair event in the chromosome.

In one aspect, provided herein is a method for detecting and/or classifying at least one structural variation and/or repair event in a chromosome from a cell, comprising the steps of: (a) generating a pair of single-stranded sister chromatids from said chromosome, wherein at least one single-stranded sister chromatid from the pair, comprises two or more target DNA sequences; (b) after step (a) contacting one or both single-stranded sister chromatid with a stain in a metaphase spread generated from the cell; (c) after step (a) contacting one or both single-stranded sister chromatid with two or more directional genomic hybridization (dGH) probes in the metaphase spread, wherein each dGH probe comprises a pool of single stranded oligonucleotides complementary to at least a portion of one of the two or more target DNA sequences, wherein each of the two or more dGH probes comprises at least one label, wherein at least two of the two or more dGH probes each binds to a different target DNA sequence on one of the single-stranded sister chromatids, and each comprises a label of a different color; (d) detecting a staining pattern of one or both single-stranded sister chromatid, wherein the staining pattern is generated based on binding of the stain to the one or both single-stranded sister chromatid; (e) generating a fluorescence pattern for one or both single-stranded sister chromatids using fluorescence detection, wherein the fluorescence pattern is based on a hybridization pattern of the at least two dGH probes to one or both single-stranded sister chromatids of the pair; (f) comparing the staining pattern of one or both single-stranded sister chromatid of step (d) to a reference staining pattern representing a control sequence; and further comparing the fluorescence pattern of step (e) to a reference fluorescence pattern representing the control sequence; and (g) determining, based on at least one staining difference between the staining pattern of one or both single-stranded sister chromatid of step (d) and the reference staining pattern and further based on at least one difference in the fluorescence pattern for one or both single-stranded sister chromatids using fluorescence detection of step (e) and the reference fluorescence pattern, the presence of the at least one structural variation and/or repair event in the chromosome.

In one aspect, provided herein is a method for detecting, determining, and/or classifying at least one structural feature of a chromosome from a cell, comprising the steps of: (a) generating a pair of single-stranded sister chromatids from said chromosome, wherein at least one sister chromatid of the pair comprises two or more target DNA sequence and at least one repetitive sequence; (b) contacting one or both single-stranded sister chromatid in a metaphase spread generated from the cell, with (i) one or more oligonucleotide markers complementary to one or more repetitive sequences on the single-stranded sister chromatid which are not target DNA sequences, wherein each of the one or more oligonucleotide markers comprises at least one label; and (ii) two or more directional genomic hybridization (dGH) probes, wherein each dGH probe comprises a pool of single stranded oligonucleotides complementary to at least a portion of the target DNA sequences, wherein each dGH probe comprises at least one label and wherein at least two of the dGH probes each bind to a different target DNA sequence on one of the single-stranded sister chromatids and each comprise a label of a different color; (c) generating a marker fluorescence pattern and a dGH fluorescence pattern of one or both single-stranded sister chromatids using fluorescence detection, wherein the marker fluorescence pattern is based on a marker hybridization pattern on the one or both single-stranded sister chromatid and the dGH fluorescence pattern is based on a dGH probe hybridization pattern of the at least two dGH probes on the one of the single-stranded sister chromatids; (d) comparing the marker fluorescence pattern to a reference marker fluorescence pattern representing a control and the dGH fluorescence pattern to a reference fluorescence pattern representing a control and/or comparing the marker fluorescence pattern to the dGH fluorescence pattern; and (e) determining, based on the comparing, the presence of the structural feature of the chromosome. In some embodiments, the comparing comprises comparing the marker fluorescence pattern to the reference marker fluorescence pattern and comparing the dGH fluorescence pattern to the reference dGH fluorescence pattern. In some embodiments, the structural feature of the chromosome is at least one structural variation and/or repair event.

In one aspect, provided herein is a method for identifying at least one chromosome that is the chromosomal source of extrachromosomal DNA (ECDNA) in a cell, comprising the steps of: a) contacting the ECDNA and either the chromosome or at least one single-stranded chromatid generated from the chromosome, with two or more directional genomic hybridization (dGH) probes in a metaphase spread from the cell, wherein each dGH probe comprises a fluorescent label of a set of fluorescent labels, wherein each dGH probe comprises a pool of single-stranded oligonucleotides that comprise a same fluorescent label of the set of fluorescent labels, wherein each single stranded oligonucleotide of a pool binds a different complementary DNA sequence within the same target DNA sequence, wherein the ECDNA and either the chromosome or the at least one single-stranded chromatid comprises a target DNA sequence for each of the two or more dGH probes, and wherein at least two of the two or more dGH probes comprise a fluorescent label of a different color; b) generating a fluorescence pattern of the ECDNA and a fluorescence pattern one or both single-stranded sister chromatids using fluorescence detection, wherein the fluorescence patterns are based on a hybridization pattern of the at least two dGH probes to the ECDNA and to the chromosome or the at least one single-stranded sister chromatid; c) comparing the fluorescence pattern of the ECDNA and the fluorescence pattern of the chromosome or the at least one single-stranded sister chromatid generated from the chromosome; and d) identifying, based on at least one similarity between the fluorescence pattern of the ECDNA and the fluorescence pattern of the chromosome or the one or both single-stranded sister chromatid, the at least one chromosome that is the chromosomal source of the ECDNA in the cell.

Any method herein for detecting, determining, and/or classifying in some aspects, can in some aspects in addition or instead, be a method to identify, determine and/or measure the chromosomal location of the detected, determined or classified structural feature and/or repair event. Furthermore, such methods can include a step for identifying, determining and/or measuring the chromosomal location of the structural feature and/or repair event. In illustrative embodiments, such chromosomal location will be a region of the chromosome, which for example can be determined based on analysis of one or more single-stranded chromatids generated from the chromosome.

In some embodiments of any aspects or embodiments disclosed herein that include a probe, a probe is a pool of single-stranded oligonucleotides. In some embodiments of methods as disclosed herein, a probe can be a dGH probe. In some embodiments, a set of labeled probes can be a set of dGH probes. In some embodiments, a dGH probe can comprise at least 10, 20, 50, 75, 100, 200, 500, or 1,000 single-stranded oligonucleotides. In some embodiments, a probe can include for example, between 10 and 2×10⁶, 1,000 and 2×10⁶, 10-10,000,00-5,000, 100-1,000, 100-500, 200-1,000, 200-500 single-stranded oligonucleotides, each with a different nucleic acid sequence. In some embodiments, a dGH probe can comprise between 1,000 to 100,000 single stranded oligonucleotides. In some embodiments, a dGH probe can comprise between 15,000 and 50,000 single-stranded oligonucleotides. In some embodiments, a dGH probe can comprise between 15,000 and 40,000 single-stranded oligonucleotides. In some embodiments, a dGH probe can comprise between 20,000 and 30,000 single-stranded oligonucleotides. In illustrative embodiments, a dGH probe can comprise between 20,000 and 50,000 single-stranded oligonucleotides. In some embodiments of any of the aspects or embodiments that include a probe or a dGH probe that comprises a pool of single-stranded oligonucleotides, such a pool of single-stranded oligonucleotides comprises single-stranded oligonucleotides of 5 to 150, 10 to 140, 15 to 130, 20 to 120, 25 to 110, 25 to 100, 25 to 90, 25 to 80, 25 to 75, 30 to 70, 30 to 65, 30 to 60, 30 to 50, or 37 to 47, 37 to 45, or 37 to 43 nucleotides in length. In some embodiments, a pool of single stranded oligonucleotides of each of the dGH probe can range in number of oligonucleotides from between 10 to 2×10⁶, 100 to 2×10⁶, 500 to 2×10⁶, 750 to 2×10⁶, 1,000 to 2×10⁶, 2,000 to 2×10⁶, 4,000 to 2×10⁶, 5,000 to 2×10⁶, 6,000 to 2×10⁶, 7,500 to 2×10⁶, 8,500 to 2×10⁶, 9,000 to 2×10⁶, 10,000 to 2×10⁶, 10,000 to 2×10⁵, 15,000 to 2×10⁵, 20,000 to 2×10⁵, 20,000 to 1×10⁵, 22,000 to 1×10⁵, 24,000 to 1×10⁵, 25,000 to 1×10⁵, 10,000 to 90,000, 10,000 to 85,000, 10,000 to 80,000, 10,000 to 75,000, 10,000 to 70,000, 20,000 to 70,000, 25,000 to 70,000, 25,000 to 65,000, 25,000 to 60,000, 25,000 to 50,000, 10 to 10,000, 20 to 10,000, 40 to 10,000, 50 to 10,000, 75 to 10,000, 100 to 5,000, 100 to 1,000, 100 to 500, 200 to 1,000, or 200 to 500.

In some embodiments, methods herein detect, identify or determine the presence of a structural variation and/or a repair event in a chromosome from a cell. In some embodiments a repair event is selected from the group consisting of a sister chromatid exchange, a sister chromatid recombination, and a combination thereof. In some illustrative embodiments, a repair event is a sister chromatid exchange. In some embodiments, a structural variation is selected from the group consisting of a change in the copy number of a segment of the chromosome, a change in the copy number of the chromosome, an inversion, a translocation, a sister chromatid recombination, a micronuclei formation, a chromothripsis event and any combination thereof. In some embodiments a structural variation is detected, and wherein the structural variation is a change in the copy number of a segment of the chromosome and the change is selected from the group consisting of an amplification, a deletion and any combination thereof. In some embodiments, a structural variation is selected from the group consisting of a change in the copy number of a segment of the chromosome, a change in the copy number of the chromosome, an insertion, a deletion, an inversion, a balanced translocation, an unbalanced translocation, a sister chromatid recombination, a micronuclei formation, a chromothripsis event, a loss or gain of genetic material, a loss or gain of one or more entire chromosome and any combination thereof. In some embodiments, a structural variation is selected from the group consisting of a change in the copy number of a segment of the chromosome, a change in the copy number of the chromosome, an insertion, a deletion, an inversion, a balanced translocation, an unbalanced translocation, a sister chromatid recombination, a micronuclei formation, a chromothripsis event, a loss or gain of genetic material, a loss or gain of one or more entire chromosome and any combination thereof. In some embodiments, the structural variation is an insertion. In some embodiments, the structural variation is a deletion. In some embodiments, the structural variation is an inversion. In some embodiments, the structural variation is a balanced translocation. In some embodiments, the structural variation is an unbalanced translocation. In some embodiments, the structural variation is a sister chromatid recombination. In some embodiments, the structural variation is a micronuclei formation. In some embodiments, the structural variation is a chromothripsis event. In some embodiments, a structural variation is numerical variation. In some embodiments, the numerical variant comprises a change in the copy number of a segment of the chromosome. In some embodiments, the numerical variation is a change in the copy number of the chromosome. In some embodiments, the numerical variation is a loss or gain of genetic material. In some embodiments, the numerical variation is a loss or gain of one or more entire chromosome. In some embodiments, there is more than one structural variation. In some embodiments, there is both a repair event and structural variation. In some embodiments, there is a structural variation within normal repair event. In some embodiments, there is a structural variation within a structural variation. In some embodiments, the structural variation is a mis-repair event. In some embodiments of any aspects or embodiments disclosed herein that include a method for detecting a repair event in a chromosome from a cell that comprises using dGH probes, a repair event can comprise an SCE. In some embodiments, a repair event can comprise a sister chromatid recombination.

In some embodiments of any of the aspects or embodiments as disclosed herein that include a probe or a dGH probe, said probe or dGH probe comprises a pool of single-stranded oligonucleotides such that each of the single-stranded oligonucleotides comprise a label, such as, but not limiting to, a fluorescent label, and are complementary to a different complementary DNA sequence within a same target DNA sequence on at least one of single-stranded sister chromatid. In some embodiments, the fluorescent label comprises one of two or more fluorescent dyes conjugated at the 5′ end of the single-stranded oligonucleotide. In some embodiments, pools of single-stranded oligonucleotides comprise labels of at least 2, 3, 4, 5, 6, 7, 8, 9, and 10 different colors, thus capable of emitting light at least 2, 3, 4, 5, 6, 7, 8, 9, and 10 different colors. In some embodiments, pools of single-stranded oligonucleotides comprise labels of 2 to 10, 3 to 10, 3 to 8, 3 to 7, or 3 to 6 different colors, thus capable of emitting light at 2 to 10, 3 to 10, 3 to 8, 3 to 7, or 3 to 6 different colors. In some embodiments, dGH probes complementary to a target DNA sequence on each single-stranded sister chromatid comprise labels of, thus capable of emitting light at, between 2 to 10, 3 to 10, 3 to 8, 3 to 6, or 4 to 7 different colors. In some embodiments, the different colors as disclosed herein appear adjacent to each other in a banded pattern or a dGH banded pattern. In some embodiments, a banded pattern or a dGH banded pattern comprises consecutive bands of different colors along a chromosome or typically a single-stranded sister chromatid. In some embodiments of any of the aspects or embodiments herein, the pools of the single-stranded oligonucleotides complementary to said two or more target DNA sequence on at least one of said single-stranded sister chromatid comprise labels of at least three different colors. In some embodiments, the dGH probes complementary to said target DNA sequence on each single-stranded sister chromatid comprise labels of between 2 and 10 different colors.

In some embodiments, a label is selected from the group consisting of a label on the end of the probe, a label on the side of the probe, one or more labels on the body of the probe, and any combination thereof. In some embodiments, a label is a body label on a sugar or amidite functional group of the probe. In some embodiments, a label is selected from the group consisting of a label detectable in the visible light spectrum, a label detectable in the infra-red light spectrum, a label detectable in the ultra violet light spectrum, and any combination thereof. In some embodiments of any of the aspects or embodiments herein, the label, the at least one label, or the fluorescent label is selected from the group consisting of a label detectable in the visible light spectrum, a label detectable in the infra-red light spectrum, a label detectable in the ultra violet light spectrum, and any combination thereof. In some illustrative embodiments, methods as disclosed herein comprise a label that is detectable in the visible light spectrum.

In some embodiments of any of the aspects or embodiments as disclosed herein that include generating a fluorescence pattern or a dGH fluorescence pattern, said fluorescence pattern or a dGH fluorescence pattern is generated using measurements of fluorescent wavelength intensities. In some embodiments, a fluorescence pattern or a dGH fluorescence pattern is generated using spectral intensity measurements along one or both single-stranded sister chromatids. In some embodiments, a fluorescence pattern or a dGH fluorescence pattern is a spectral fingerprint of the one or both single-stranded sister chromatids. In some embodiments, a fluorescence pattern or a dGH fluorescence pattern is a spectral profile. In some embodiments, a spectral profile specifically excludes one or more spectral regions of the spectral profile. In some embodiments, a fluorescence pattern or a dGH fluorescence pattern specifically excludes one or more spectral regions of the spectral profile. In some embodiments, a reference fluorescence pattern representing a control sequence comprising spectral intensity measurements along the one or both single-stranded sister chromatids is used in the methods for comparison with a fluorescence pattern or a dGH fluorescence pattern as disclosed herein. In some embodiments, an oligonucleotide density along the one or both single-stranded sister chromatids is used in the detecting the structural variation and/or the repair event. In some embodiments, a fluorescence pattern or a dGH fluorescence pattern is used for detecting at least one structural feature, at least one structural variation and/or repair event, or for identifying at least one chromosome that is the chromosomal source of extrachromosomal DNA (ECDNA) in a cell with the aid of, by using, by running a computer program that performs, or by running a computer program whose code is based at least in part on, artificial intelligence. In some embodiments, generating a fluorescence pattern or a dGH fluorescence pattern comprises use of narrow band filters and processing of spectral information with software. In some embodiments, the comparing of the fluorescence pattern, spectral measurements, or spectral profile comprises spectral analysis of the bleeding of at least one band over at least one other band on the same chromosome or same single stranded sister chromatid.

In some embodiments of any of the aspects or embodiments herein, the target of the preliminary dGH probe, the first DNA sequence of the first dGH probe, or the second target DNA sequence of the second dGH probe, comprise a location selected from a telomeric, a subtelomeric and a centromeric region of the chromosome or single stranded sister chromatid. In some embodiments, the hybridizing or the contacting comprises two or more dGH probes, each dGH probe comprising a fluorescent label of a set of fluorescent labels, wherein at least two of the two or more dGH probes each binds to a different target DNA sequence on the target chromosome and each comprises a fluorescent label of a different color.

In some embodiments of any of the aspects or embodiments as disclosed herein that include generating a fluorescence pattern, or a dGH fluorescence pattern and comparing the same with a reference fluorescence pattern, a fluorescence pattern or a dGH fluorescence pattern of one or both single-stranded sister chromatids is of one single-stranded sister chromatid and the reference fluorescence pattern is of the other single-stranded sister chromatid. In some embodiments, a fluorescence pattern or a dGH fluorescence pattern of one or both single-stranded sister chromatids is of one single-stranded sister chromatid and the reference fluorescence pattern is of a homolog of the chromosome from the cell. In some embodiments, a fluorescence pattern or a dGH fluorescence pattern is of one single-stranded sister chromatid and the reference fluorescence pattern is of the other single-stranded sister chromatid. In some embodiments, a reference fluorescence pattern lacks said at least one structural variation or repair event. In some embodiments, a reference fluorescence pattern comprises said at least one structural variation or repair event. In some embodiments, a reference fluorescence pattern comprises an intentional distribution of labeled dGH probes.

In some embodiments of any of the aspects or embodiments as disclosed herein that include contacting probes or dGH probes to a target DNA sequence or sequences found on one or both single-stranded sister chromatids, target DNA sequence or sequences are between 1 Kb and 150 Mb, 1 Kb and 100 Mb, 1 Kb and 50 Mb, 1 Kb and 30 Mb, 1 Kb and 25 Mb, 1 Kb and 10 Mb, 1 Kb and 1 Mb, 1 Kb and 100 Kb, 1 Kb and 10 Kb, 1 Kb and 5 Kb, 2 Kb and 150 Mb, 2 Kb and 100 Mb, 2 Kb and 50 Mb, 2 Kb and 30 Mb, 2 Kb and 25 Mb, 2 Kb and 10 Mb, 21 Kb and 1 Mb, 2 Kb and 100 Kb, 2 Kb and 10 Kb, 2 Kb and 5 Kb, 10 Kb and 150 Mb, 10 Kb and 100 Mb, 10 Kb and 50 Mb, 10 Kb and 30 Mb, 10 Kb and 25 Mb, 10 Kb and 10 Mb, 10 Kb and 1 Mb, 10 Kb and 100 Kb, 10 Kb and 50 Kb, 10 Kb and 25 Kb, 1 Mb and 150 Mb, 1 Mb and 100 Mb, 1 Mb and 50 Mb, 1 Mb and 30 Mb, 1 Mb and 25 Mb, 1 Mb and 10 Mb, 1 Mb and 5 Mb, 5 Mb and 150 Mb, 5 Mb and 100 Mb, 5 Mb and 50 Mb, 5 Mb and 30 Mb, 5 Mb and 25 Mb, or 5 Mb and 10 Mb. In some embodiments, target DNA sequences bound by each of the at least two or more dGH probes are consecutive target DNA sequences on one of the single-stranded sister chromatids, such that a multi-colored consecutive banding pattern is generated on the one of the single stranded sister chromatids, and wherein bands of 2,000 nucleotides in length can be detected and used in the detecting or determining steps. In illustrative embodiments, a multi-colored consecutive banding pattern is generated on the one of the single stranded sister chromatids, and wherein bands of 2,000 nucleotides in length are used in the detecting or determining steps. In some embodiments, bands having a size below 5,000, 4,000, 3,000, 2,000, or 1,000 nucleotides in length can be detected and used in the detecting or determining steps. In some embodiments, bands of 1,000 nucleotides in length are used in the detecting or determining steps. In some embodiments, bands of between 5,000 to 1,000, 4,500 to 1,000, 4,000 to 1,000, 3,500 to 1,000, 3,000 to 1,000, or 2,000 to 1,000 nucleotides in length are used in the detecting or determining steps. In some embodiments, banding pattern comprises individual bands ranging in size from between 1 Kb and 150 Mb, 1 Kb and 100 Mb, 1 Kb and 50 Mb, 1 Kb and 30 Mb, 1 Kb and 25 Mb, 1 Kb and 10 Mb, 1 Kb and 1 Mb, 1 Kb and 100 Kb, 1 Kb and 10 Kb, 1 Kb and 5 Kb, 2 Kb and 150 Mb, 2 Kb and 100 Mb, 2 Kb and 50 Mb, 2 Kb and 30 Mb, 2 Kb and 25 Mb, 2 Kb and 10 Mb, 21 Kb and 1 Mb, 2 Kb and 100 Kb, 2 Kb and 10 Kb, 2 Kb and 5 Kb, 10 Kb and 150 Mb, 10 Kb and 100 Mb, 10 Kb and 50 Mb, 10 Kb and 30 Mb, 10 Kb and 25 Mb, 10 Kb and 10 Mb, 10 Kb and 1 Mb, 10 Kb and 100 Kb, 10 Kb and 50 Kb, 10 Kb and 25 Kb, 1 Mb and 150 Mb, 1 Mb and 100 Mb, 1 Mb and 50 Mb, 1 Mb and 30 Mb, 1 Mb and 25 Mb, 1 Mb and 10 Mb, 1 Mb and 5 Mb, 5 Mb and 150 Mb, 5 Mb and 100 Mb, 5 Mb and 50 Mb, 5 Mb and 30 Mb, 5 Mb and 25 Mb, or 5 Mb and 10 Mb. In some embodiments, banding pattern comprises individual bands ranging in size from between 1 Kb and 100 Kb, 1 Kb and 10 Kb, 2 Kb and 100 Kb, or 2 Kb and 10 Kb. In some embodiments, banding pattern comprises individual bands that range in size from between 1 Mb and 30 Mb, 1 Mb and 25 Mb, 1 Mb and 10 Mb, 1 Mb and 5 Mb, 5 Mb and 30 Mb, 5 Mb and 25 Mb, or 5 Mb and 10 Mb. In some embodiments, dGH probes as disclosed herein are designed to provide a banding pattern comprising individual bands that range in size from between 1 Kb and 150 Mb, 1 Kb and 100 Mb, 1 Kb and 50 Mb, 1 Kb and 30 Mb, 1 Kb and 25 Mb, 1 Kb and 10 Mb, 1 Kb and 1 Mb, 1 Kb and 100 Kb, 1 Kb and 10 Kb, 1 Kb and 5 Kb, 2 Kb and 150 Mb, 2 Kb and 100 Mb, 2 Kb and 50 Mb, 2 Kb and 30 Mb, 2 Kb and 25 Mb, 2 Kb and 10 Mb, 21 Kb and 1 Mb, 2 Kb and 100 Kb, 2 Kb and 10 Kb, 2 Kb and 5 Kb, 10 Kb and 150 Mb, 10 Kb and 100 Mb, 10 Kb and 50 Mb, 10 Kb and 30 Mb, 10 Kb and 25 Mb, 10 Kb and 10 Mb, 10 Kb and 1 Mb, 10 Kb and 100 Kb, 10 Kb and 50 Kb, 10 Kb and 25 Kb, 1 Mb and 150 Mb, 1 Mb and 100 Mb, 1 Mb and 50 Mb, 1 Mb and 30 Mb, 1 Mb and 25 Mb, 1 Mb and 10 Mb, 1 Mb and 5 Mb, 5 Mb and 150 Mb, 5 Mb and 100 Mb, 5 Mb and 50 Mb, 5 Mb and 30 Mb, 5 Mb and 25 Mb, or 5 Mb and 10 Mb.

In some embodiments of any of the aspects or embodiments that include a fluorescence pattern or a dGH fluorescence pattern that represents a banding pattern, such a banding pattern comprises individual bands of different colors, and wherein individual bands of 1,000 bases in length can be detected and used in the detecting or determining steps. In some embodiments, the fluorescence pattern represents a banding pattern comprising bands of different colors, and wherein individual bands of 2,000 bases in length can be detected. In some embodiments, individual bands of 2,000, 3,000, 4,000, or 5,000 bases in length can be detected and used in the detecting or determining steps. In some embodiments, individual bands that range in size from 1,000 to 5,000 bases, 1,000 to 4,500 bases, 1,000 to 4,000 bases, 2,000 to 5,000 bases, or 2,000 to 4,000 bases can be detected and used in the detecting or determining steps. In some embodiments, methods as disclosed herein are capable of resolving fluorescence patterns generated from target sequences that are as small as 2,000 bases. In some embodiments, methods as disclosed herein are capable of resolving fluorescence patterns generated from target sequences that are as small as 1,000 bases. In some embodiments, methods as disclosed herein are capable of performing the detecting or determining using fluorescence patterns generated from target sequences that are as small as 5,000, 4,000, 3,000, 2,000 or 1,000 bases. In some embodiments, methods as disclosed herein are capable of performing the detecting or determining using fluorescence patterns generated from target sequences that range in size from 1,000 to 5,000 bases, 1,000 to 4,500 bases, 1,000 to 4,000 bases, 2,000 to 5,000 bases, or 2,000 to 4,000 bases.

In some embodiments, the cell is from a test population of cells. The test population of cells, and thus the cell, can comprise genetically modified cells having a recombinant nucleic acid insert and/or an edited site. In some embodiments, the recombinant nucleic acid insert comprises a chimeric antigen receptor sequence, a transgenic sequence, a gene-edited sequence, a deleted gene sequence, an inserted gene sequence, a DNA sequence for binding guide RNA, a transcription activator-like effector binding sequence, or a zinc finger binding sequence. In some embodiments, the recombinant nucleic acid insert comprises a transgene. In some embodiments, the transgene is a chimeric antigen receptor sequence. In some embodiments, the transgene is a gene-edited sequence. In some embodiments, a set of multi-color dGH probes is used wherein at least one target DNA sequence for a probe of the set includes a target site for gene editing.

In some embodiments of any of the aspects or embodiments that include a method of detecting using a probe or a dGH probe, after the step of generating a pair of single-stranded sister chromatids from said chromosome, or performing dGH reaction further comprises contacting the single-stranded sister chromatid with oligonucleotide markers complementary to repetitive sequences on the single-stranded sister chromatid which are not target DNA sequences, wherein each of the oligonucleotide markers comprises at least one label; detecting a marker hybridization pattern of the sister chromatid; comparing the marker hybridization pattern to a reference marker hybridization pattern representing a control; and determining the presence of the at least one structural variation and/or repair event based in part on at least one marker hybridization pattern difference between the marker hybridization pattern of the sister chromatid and the reference marker hybridization pattern. In some embodiments, a reference marker hybridization pattern lacks said at least one structural variation or repair event. In some embodiments, a reference marker hybridization pattern comprises said at least one structural variation or repair event. In some embodiments, a reference marker hybridization pattern comprises an intentional distribution of labeled dGH probes.

In some embodiments of any of the aspects or embodiments that include a method of detecting using a probe or a dGH probe, further comprising contacting the single-stranded sister chromatid with a stain; detecting a staining pattern of the sister chromatid; comparing the staining pattern to a reference staining pattern representing a control; and determining the presence of the at least one structural variation based in part on at least one staining difference between the staining pattern of the sister chromatid and the reference staining pattern. In some embodiments, a staining pattern obtained as per any of the aspects or embodiments as disclosed herein, is of one single-stranded sister chromatid and the reference staining pattern is of the other single-stranded sister chromatid. In some embodiments, a staining pattern is of one single-stranded sister chromatid and the reference staining pattern is of a normal homolog of the chromosome. In some embodiments, the stain with which a staining patter is obtained is selected from the group consisting of DAPI, Hoechst 33258, and Actinomycin D. In some embodiments that include a reference staining pattern, such a reference staining pattern lacks said at least one structural variation or repair event. In some embodiments, a reference staining pattern comprises said at least one structural variation or repair event.

In some embodiments of any of the aspects or embodiments that include a method that uses a dGH probe for generating a banding pattern, fluorescence pattern, or a dGH fluorescence pattern, the target DNA sequences bound by each of the two or more dGH probes are consecutive target DNA sequences on one of the single-stranded sister chromatids, such that a multi-colored consecutive banding pattern is generated on the one of the single stranded sister chromatids. In some embodiments, such a fluorescence pattern or a dGH fluorescence pattern is a banding pattern on the at least one single-stranded sister chromatid comprising bands of different colors that are detected using a fluorescence microscope system. In some embodiments, a banding pattern on the at least one single-stranded sister chromatid comprises bands of between 2 and 15, 2 and 14, 2 and 12, 2 and 10, 2 and 8, 2 and 6, or 2 and 5 different colors. In some embodiments, the banding pattern on the at least one single-stranded sister chromatid comprises bands of between 2 and 10 different colors. In some illustrative embodiments, between 2 and 5 different colors are used to generate a multi-colored banding pattern.

In some embodiments of any of the aspects or embodiments that include a method for detecting at least one structural feature, structural variation, or repair event of a chromosome of a cell, or a method for identifying at least one chromosome that is the chromosomal source of extrachromosomal DNA (ECDNA) in a cell, at least one single-stranded sister chromatid is at least between 20 and 23 single-stranded sister chromatids derived from one or more copies of between 20 and 23 different human chromosomes from the cell. In some embodiments of any aspects or embodiments herein, the at least one single-stranded sister chromatid is at least between 20 and 23 single-stranded sister chromatids derived from one or more copies of between 20 and 23 different human chromosomes from the cell. In some embodiments, the different human chromosomes do not include a Y chromosome. In some embodiments, at least one single-stranded sister chromatid are single-stranded sister chromatids derived from every human chromosome from the cell. In some embodiments, at least one single-stranded sister chromatid are single-stranded sister chromatids derived from every human chromosome from the cell except the Y chromosome.

In some embodiments, the contacting of at least one chromosome or at least one single stranded sister chromatid of a chromosome with two or more dGH probes comprises embedding a sample comprising the at least one chromosome or at least one single stranded sister chromatid of a chromosome in a swellable hydrogel and chemically linking the sample to the hydrogel, further wherein the hydrogel is swelled to increase spatial resolution across the x, y, and z axes.

In some embodiments of any of the aspects and embodiments that include a method for detecting at least one structural feature, structural variation, or repair event of a chromosome of a cell, or a method for identifying at least one chromosome that is the chromosomal source of extrachromosomal DNA (ECDNA) in a cell, during the contacting, the one or both single-stranded sister chromatids or another single-stranded sister chromatid is contacted with an internal control dGH probe ladder comprising a control set of at least 3 control dGH probes that bind to target DNA sequences on a control single-stranded sister chromatid, wherein the control single-stranded sister chromatid is one of the one or both single stranded sister chromatids or the other single-stranded sister chromatid, wherein the control single-stranded sister chromatid is not the single-stranded sister chromatid from which the fluorescence pattern is generated and detected to detect the presence of the structural variant and/or repair event, and wherein the control dGH probes:
i) each have a different number of single-stranded oligonucleotides,
ii) each have a number of single stranded oligonucleotides that is within 10, 9, 8, 7, 6, 5, 4, 3, 2, 1 of each other, or equal to each other, and each binds a control target DNA sequence whose length that differs for each control dGH probe of the ladder, for example by 1 MB, 2 MB, 3 MB, 4 MB, 5 MB, or 10 MB,
iii) each have the same number of oligonucleotides spread out evenly or unevenly across a target DNA sequence of a variable target size; for example 10-1,000, 500, 250, 200, or 100 oligonucleotides, or 50-150 or 100 oligonucleotides, 75-100 oligonucleotides, 80-100 oligonucleotides, 85-95 oligonucleotides, or 90 oligonucleotides spread our evenly or non-evenly across within a target DNA sequence of between 5 kb and 100 kb, or 6 kb and 50 kb, or 5 kb and 10 kb, or 6 kb, 12 kb, 18 kb, or 24 kb; and/or
iv) each binds to a target DNA sequence that is spaced out at different known distances on the control single-stranded chromatid. In some embodiments, the control dGH probes each have a different number of single-stranded oligonucleotides, and wherein a control fluorescence pattern is used to determine a limit of detection of a particular performance of the method. In some embodiments, the control dGH probes each have a number of single stranded oligonucleotides that is within 10, 9, 8, 7, 6, 5, 4, 3, 2, 1 of each other, and each binds a control target DNA sequence whose length that differs for each control dGH probe of the ladder, for example by 1 MB, 2 MB, 3 MB, 4 MB, 5 MB, or 10 MB, and wherein a control fluorescence pattern is used to determine a limit of detection of a particular performance of the method. In some embodiments, the control dGH probes each bind to a target DNA sequence that is spaced out at different known distances on the control single-stranded chromatid, and wherein a control fluorescence pattern is used to determine the resolvability of two bands on a single-stranded sister chromatid for a particular performance of the method. In illustrative embodiments in methods that include the internal control dGH ladder, the methods further comprise generating a control fluorescent pattern from the control single-stranded sister chromatid using fluorescence detection, wherein the control fluorescence pattern is based on a hybridization pattern of the control dGH probes to the control single-stranded sister chromatid.

In some embodiments of any of the aspects and embodiments that include a method for detecting at least one structural feature, structural variation, or repair event of a chromosome of a cell, or a method for identifying at least one chromosome that is the chromosomal source of extrachromosomal DNA (ECDNA) in a cell, further comprises measuring the level of condensation of the one or more single-stranded chromatids in the metaphase spread. In some embodiments, the method further comprises using the level of condensation of the one or more single-stranded sister chromatids to determine the resolution of the detection of a structural feature, structural variation, and/or repair event. In some embodiments, the level of condensation is factored into, affects, used, taken into account, considered, or otherwise utilized in the determining or detecting. In some embodiments, the method further comprises reporting the results of the detecting or determining. In some embodiments, reporting includes reporting the level of chromosome condensation in the metaphase spread for the one or more single-stranded sister chromatids. In some embodiments, the method is capable of resolving the location of the structural feature on the chromosome to within a 2 Mb, 1 Mb, 500 Kb, 250 Kb, 200 Kb, or 100 Kb region of the chromosome. In some embodiments, the cell is incubated with an intercalating agent before the pair of single-stranded chromatids are contacted with the two or more dGH probes in the metaphase spread. In some embodiments, the method is capable of resolving the location of the structural feature on the chromosome within the range of 5 Mb to 100 Kb, 4 Mb to 100 Kb, 3 Mb to 100 Kb, 2 Mb to 100 Kb, or 1 Mb to 100 Kb region. In some illustrative embodiments, the method is capable of resolving the location of the structural feature on the chromosome to within a 1 Mb region of the chromosome.

In some embodiments of any of the aspects and embodiments that include a method for identifying at least one chromosome that is the chromosomal source of extrachromosomal DNA (ECDNA) in a cell, further comprises, based on the comparing step, identifying a position on the at least one chromosome or at least one single stranded sister chromatid of a chromosome from which DNA in the ECDNA originated. In some embodiments, the origination of ECDNA from the at least one chromosome or at least one single stranded sister chromatid of a chromosome was caused by an amplification of DNA at the position. In some embodiments, at least one oncogene is identified on the ECDNA. In some embodiments, the ECDNA is selected from the group consisting of episomal DNA and vector-incorporated DNA. In some embodiments, at least one target area on at least one chromosome or at least one single stranded sister chromatid of a chromosome is identified for target enrichment and at least one chromosome or at least one single stranded sister chromatid of a chromosome is contacted with target enrichment probes.

In some embodiments of any of the aspects and embodiments that include a method for detecting at least one structural feature, structural variation, or repair event of a chromosome of a cell, or a method for identifying at least one chromosome that is the chromosomal source of extrachromosomal DNA (ECDNA) in a cell, such a method is a computer implemented method. In some embodiments, the some or all of the performing, the generating, the comparing, the detecting, and/or the determining are computed with a computer system. In some embodiments, the detecting or the determining is performed using a computer system. In some embodiments, some or all of the performing, the generating, the comparing, the detecting, and/or the determining are implemented by a computer processor. In some embodiments, the determining is implemented by the computer processor, and comprises:
(a) receiving the fluorescence pattern representing at least one sequence of bases on a subject DNA strand, the fluorescence pattern including frequency data corresponding to the sequence of bases on the subject DNA strand, the frequency data including at least two color channels;
(b) converting the fluorescence pattern to a data table for the subject DNA strand, the data table comprising positional data and intensity data for the at least two color channels for the sequence of bases; and
(c) storing the data table to a memory. In some embodiments, wherein the fluorescence pattern is a spectral profile.

In some embodiments of any of the aspects or embodiments herein, prior to contacting, single-stranded sister chromatids (i.e. each single-stranded sister chromatid) are prepared, for example from the metaphase-enriched cell population, by degrading chromosome strands (e.g. double-stranded chromatids). In some subembodiments of the immediately above embodiments, the degrading is performed by incorporating a DNA analog into genomic DNA of individual cells of the population of cells for one cell cycle, and stripping or degrading the newly synthesized chromosome strand (i.e. chromatid strand) that incorporated the DNA analog, which in some embodiments is a uridine analog, for example BrdU. In some subembodiments of methods that comprise incorporation of a uriding analog, the method further comprises staining the single-stranded sister chromatids with a DNA stain, which in illustrative subembodiments is an intercalating dye that preferentially binds to double-stranded DNA at A-T sites. Such a DNA stain in some embodiments is a bis-benzimide, such as a Hoechst stain.

In some embodiments of any of the aspects or embodiments herein, prior to contacting, single-stranded chromatids are prepared by denaturing the chromosomes from the metaphase-enriched cell population. In some embodiments of any of the aspects or embodiments herein, the denaturing is a temperature-induced denaturation. In some embodiments, of any of the aspects or embodiments herein, the cells are a metaphase-enriched cell population, or the chromosomes are from a metaphase-enriched cell population.

In some embodiments of any of the aspects or embodiments herein, the generating comprises preparing single-stranded sister chromatids from the metaphase-enriched cell population by degrading a chromosome strand, typically a newly synthesized chromosome strand. In some embodiments of any of the aspects or embodiments herein, the degrading is performed by incorporating a DNA analog into the individual cells of the population of cells for one cell cycle, and degrading or stripping the newly synthesized chromosome strand that incorporates the DNA analog. In some embodiments of any of the aspects or embodiments herein, the generating comprises preparing single-stranded chromatids by denaturing the chromosomes from the metaphase-enriched cell population, and in certain subembodiments, the denaturing is temperature-induced denaturation.

In some embodiments, of any of the aspects or embodiments herein, the cells are derived from cell culture. In some embodiments, the cells are derived from a tissue sample.

In some embodiments, of any of the aspects or embodiments herein, at least one mRNA is detected or measured. In some embodiments, at least one protein is detected or measured.

In some embodiments, at least one nucleic acid is collected or isolated and sequenced. In some embodiments, the at least one nucleic acid is a DNA or RNA. In some embodiments, the method further comprises collecting (or isolating) single-stranded sister chromatids from the sorted cell population, or separately from individual cells of the sorted cell population, and sequencing at least one, and typically a plurality of nucleic acids generated from the collected single-stranded sister chromatids. In some embodiments, of any aspects or embodiments herein that comprise nucleic acid sequencing, the nucleic acid sequencing is single-cell template strand sequencing and/or next-generation sequencing. Thus, in one aspect provided herein is a method for analyzing a chromosome of individual cells of a population of cells, the method comprising the steps of:
a) contacting a pair of single-stranded sister chromatids in a metaphase spread prepared from individual cells of the population of cells, in illustrative embodiments a G2/metaphase or metaphase-enriched subpopulation from the population of cells, with a first dGH probe, the first dGH probe comprising a first colored fluorescent label of a set of fluorescent labels, wherein the first dGH probe comprises a pool of single-stranded oligonucleotides that comprise a same fluorescent label of the set of fluorescent labels, and wherein each single stranded oligonucleotide of a pool binds a different complementary DNA sequence within a same target DNA sequence found on one of the single-stranded sister chromatids;
b) detecting a fluorescence signal from the first colored fluorescent label, from one or both single-stranded sister chromatids;
c) collecting (or isolating) one or more, typically a population of single-stranded sister chromatids from the cell population, in illustrative embodiments a G1-enriched subpopulation from the population of cells, for example separately from individual cells or nuclei therein, of the cell population; and
d) sequencing the collected one or more, or typically the population of single-stranded sister chromatids.

Accordingly, in another aspect, provided herein is a method for detecting at least one target DNA sequence, at least one structural variation and/or at least repair event in a chromosome of individual cells of a population of cells, the method comprising the steps of:
a) contacting a pair of single-stranded sister chromatids in a metaphase spread prepared from individual cells of a G2/metaphase, or metaphase-enriched subpopulation of the population of cells, with a first dGH probe, the first dGH probe comprising a first colored fluorescent label of a set of fluorescent labels, wherein the first dGH probe comprises a pool of single-stranded oligonucleotides that comprise a same fluorescent label of the set of fluorescent labels, wherein each single stranded oligonucleotide of a pool binds a different complementary DNA sequence within a same target DNA sequence found on one of the single-stranded sister chromatids;
b) generating a fluorescence pattern from one or both single-stranded sister chromatids using fluorescence detection, wherein the fluorescence pattern is based on a hybridization pattern of the first dGH probe to one or both single-stranded sister chromatids of the pair;
c) collecting (or isolating) one or more, typically a population of single-stranded sister chromatids from a G1-enriched subpopulation of the population of cells, in illustrative embodiments separately for individual cells of the cell population;
d) sequencing the collected one or more, or typically the population of, single-stranded sister chromatids from the G1-enriched subpopulation; and
e) detecting based on the fluorescence pattern and/or based on the sequencing, the presence of the at least one structural variation in the chromosome from individual cells. In some illustrative embodiments, the detecting is based on both the fluorescence pattern and the sequencing.

In these aspects that include both generating a fluorescence pattern and sequencing, the sorting is typically performed using fluorescence-based sorting. Such fluorescence-based sorting can include separately isolating the G2/M or metaphase-enriched subpopulation using a G2/M fluorescence gate and the G1-enriched subpopulation using a G1 fluorescence gate.

In illustrative embodiments of these aspects that include both generating a fluorescence pattern and sequencing, single-stranded sister chromatids (i.e. each single-stranded sister chromatid) are prepared, for example after a single cell division and/or from a G2/M or metaphase-enriched cell subpopulation and a G1-enriched cell subpopulation, by degrading chromosome strands (e.g. from double-stranded chromatids). The degrading can be performed by incorporating a DNA analog into genomic DNA of individual cells of a population of cells for one cell cycle, and degrading the newly synthesized chromosome strand (i.e. chromatid strand) that incorporated the DNA analog, which in some embodiments is a uridine and/or a cytidine analog, for example BrdU/BrdC. In some subembodiments of methods that comprise incorporation of a uriding analog, the method further comprises staining the single-stranded sister chromatids with a DNA stain, which in illustrative subembodiments is an intercalating dye that preferentially or exclusively binds to double-stranded DNA at A-T sites. Such a DNA stain in some embodiments is a bis-benzimide stain, such as a Hoechst stain (e.g. Hoechst 33258 or Hoechst 33342). Furthermore, in some embodiments, such aspects include preparing a control population that is not incubated with the nucleotide analog.

In some embodiments, of any of the aspects or embodiments herein, the DNA is stretched. In some embodiments, the isolated DNA is stretched. In some embodiments, of any aspects or embodiments herein, that comprise one or more partitions, the one or more partitions comprise isolated DNA and/or isolated RNA.

In some embodiments, the partitioned chromosomes are derived from the cell sample. In some embodiments, the partitioned chromosomes are derived from more than one cell sample.

In some embodiments, of any aspects or embodiments herein, the two or more dGH probes are the same for each partition. In some embodiments, the two or more oligonucleotide probes in a first partition comprise at least one difference in nucleic acid sequences in comparison to the two or more oligonucleotide probes in a second partition.

In some embodiments, of any aspects or embodiments herein, the chromosomes are derived from cell culture. In some embodiments, the chromosomes are derived from a tissue sample.

In some embodiments, of any aspects or embodiments herein, that comprise a spatial arrangement, the spatial arrangement is a grid. In some embodiments, the grid provides for a pre-determined hybridization pattern using one or more consistent hybridization probes across specified X and Y coordinates. In some embodiments, the pre-determined hybridization pattern is different at adjacent grid locations, as defined by the specified X and Y coordinates.

In some embodiments, of any aspects or embodiments herein, the generating the fluorescence pattern comprises use of narrow band filters and processing of spectral information with software.

In some embodiments, of any aspects or embodiments herein, the permeabilizing conditions comprise sonication, electroporation, or contacting the cells with the first dGH probe in the presence of a transfection agent under effective conditions by which the first dGH probe enters the cell and the nucleus of the cell.

In some embodiments, of any aspects or embodiments herein, the method further comprises lysing the cells before the hybridizing or the generating.

In some embodiments, of any aspects or embodiments herein, the first dGH probe binds a target DNA sequence on the target chromosome that encompasses at least 25, 50, 75, 90, 90, 95, 96, 97, 98, or 99% of a chromosome. In some embodiments, the first dGH probe binds a target DNA sequence on the target chromosome that encompasses at least 25, 50, 75, 90, 90, 95, 96, 97, 98, or 99% of a chromosome other than the centromeric and telomeric regions. In some embodiments, the first dGH probe binds a target DNA sequence on the target chromosome that encompasses substantially an entire chromosome other than the centromeric and telomeric regions. In some embodiments, the first dGH probe binds a target DNA sequence on the target chromosome that encompasses substantially an entire chromosome.

In some embodiments, of any aspects or embodiments herein, applying the fluorescence sorting comprises an automated sorting method. In some embodiments, of any aspects or embodiments herein, the DNA stain is selected from the group consisting of propidium iodine, 7-AAD, Hoechst, chromomycin A3, quinacrine, daunomycin, or any other fluorochromes that bind chromosomes through DNA intercalation or that bind a secondary structure of DNA.

In some embodiments, of any aspects or embodiments herein, the two or more dGH probes are part of a set of dGH probes designed against a known gene sequence for a target disease. In some embodiments, the set of dGH probes are part of a screening panel for a disease type. In some embodiments, the screening panel comprises between 50 and 100 sets of dGH probes, depending on disease type. In some embodiments, the screening panel is a neurological disease screening panel.

In some embodiments, of any aspects or embodiments herein, the population of cells are a population of induced pluripotent stem cells (iPSCs). In some embodiments, the iPSCs are derived from a patient with an idiopathic neurodegenerative disease.

In some embodiments, of any aspects or embodiments herein, the population of cells are labeled with a cell surface antibody panel. In some embodiments, the surface antibody panel comprises antibodies to cell surface markers CD3, CD4, CD8, and CD4RA. In some embodiments, wherein the population of cells are further labeled with a Live/Dead stain. In some embodiments, the population of cells are sorted to distinguish naïve, memory and effector T cell populations. In some embodiments, the population of cells is a population of edited cells. In some embodiments, wherein the cells are CAR-T cells. In some embodiments, the population of cells are derived from a single patient. In some embodiments, the population of cells are derived from multiple patient samples.

In some embodiments, of any aspects or embodiments herein, the method comprises treating the population of cells with colchicine. In some embodiments, the step of enriching comprises treating the population of cells with N-methyl-N-deacetyl-colchicine.

In some embodiments, of any aspects or embodiments herein, the two-dimensional spatial arrangement of partitions is an array of addressable positions on a solid support. In some embodiments, the placing comprising immobilizing the chromatids on an array of addressable positions on a solid support. In some embodiments, the immobilizing is done by spotting the chromatids on the array.

In some embodiments, of any aspects or embodiments herein, the method is performed more than one time, wherein the set of single-stranded chromatids for each performance of the method are from a different sample or population of cells, and wherein the chromosomes or cells for each performance of the method are placed in a different partition or in the same addressable position in an array of positions within a partition. In some embodiments, in the two-dimensional spatial arrangement of partitions, and optionally wherein each partition comprises an array of addressable locations. In some embodiments, there are 2, 4, 6, 8, 12, 24, 48, 96, 384, 768, 1536, or 3456 partitions in the two-dimensional spatial arrangement of partitions. In some embodiments, there are 96 partitions, or a multiple thereof, in the two-dimensional spatial arrangement of partitions. In some embodiments, there are 96, 384, 768, 1536, or 3456 partitions in the two-dimensional spatial arrangement of partitions. In some embodiments, two or more individual sorted cells or chromatids are immobilized in individual partitions. In some embodiments, the method is performed more than one time, wherein the set of single-stranded chromatids for each performance of the method are from a different sample or population of cells, and wherein the chromosomes or cells for each performance of the method are placed in a different partition. In some embodiments, the performances are performed in parallel, for example using robotics, microfluidic channels, and/or multi-pipetting instruments.

In some embodiments, the first target DNA sequence is unique in the genome of the cells. In some embodiments, the complementary sequence for every probe used in the contacting is unique in the genome of the cells. In some embodiments of methods herein, nuclei are not isolated and/or nuclei are not sorted during the method.

In some embodiments of any method or array herein that includes a two-dimensional spatial arrangement of partitions, the two-dimensional spatial arrangement of partitions is an array with 4, 8, 12, 24, 48, 96, 192, or 384 partitions. In some embodiments the method is performed using single-stranded sister chromatids from at least 24, 48, 96, 192, 384 samples, and in illustrative embodiments, the single-stranded sister chromatids from each sample are contacted with one, two, three or more, or a panel of dGH probes on different partitions of the array.

In some embodiments in any aspects or embodiments herein, the solid support comprises two or more physical barrier partitions each comprising the two-dimensional, regularly spaced arrangement of the cells or chromosomes. In some embodiments, wherein the cell population is derived from more than one cell sample, and wherein the cells or the chromosomes from different samples of the more than one cell sample are placed on the solid support within different partitions. In some embodiments, wherein one physical barrier partition contains the first dGH probe and another physical barrier partition contains the second dGH probe during performance of the method. In some embodiments, wherein the two-dimensional, regularly spaced arrangement is an array with at least 96 selected, separated locations. In some embodiments, wherein cell population comprises cells from at least 96 samples, wherein cells from each sample are placed in a separate partition on the solid support. In some embodiments, wherein the array comprises single-stranded chromatids from over 40,000 metaphase-enriched cells. In some embodiments wherein the two-dimensional, regularly spaced arrangement is an array with at least 96 selected, separated locations. In some embodiments, the solid support comprises at least 6 rows of partitions, each comprising the two-dimensional, regularly spaced arrangement. In some embodiments, the solid support comprises at least 6 rows of partitions, each comprising the two-dimensional, regularly spaced arrangement.

In some embodiments of any aspects or embodiments herein, the first target DNA sequence is unique in the genome of the cells. In some embodiments, wherein the complementary sequence for every probe used in the contacting is unique in the genome of the cells. In some embodiments, the spectral profile is a banding pattern on the at least one single-stranded sister chromatid comprising bands of different colors. In some embodiments, between 2 and 10 dGH probes are used in the method and the banding pattern on the at least one single-stranded sister chromatid comprises bands of between 2 and 10 different colors. In some embodiments,

Provided herein in some aspects of any of the aspects provided herein is a program storage device readable by a computer, tangibly embodying a program of instructions executable by the computer to perform some or all of the performing, the generating, the comparing, the detecting, and/or the determining steps of any one of the embodiments and aspects that include a method as disclosed herein for detecting at least one structural feature, structural variation, or repair event of a chromosome of a cell, or a method for identifying at least one chromosome that is the chromosomal source of extrachromosomal DNA (ECDNA) in a cell.

While the embodiments of the present disclosure are amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

The following non-limiting examples are provided purely by way of illustration of exemplary embodiments, and in no way limit the scope and spirit of the present disclosure. Furthermore, it is to be understood that any aspect or embodiment disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

### EXAMPLES

### Example 1. Whole Chromosome Analysis Using Single Color Whole Chromosome dGH

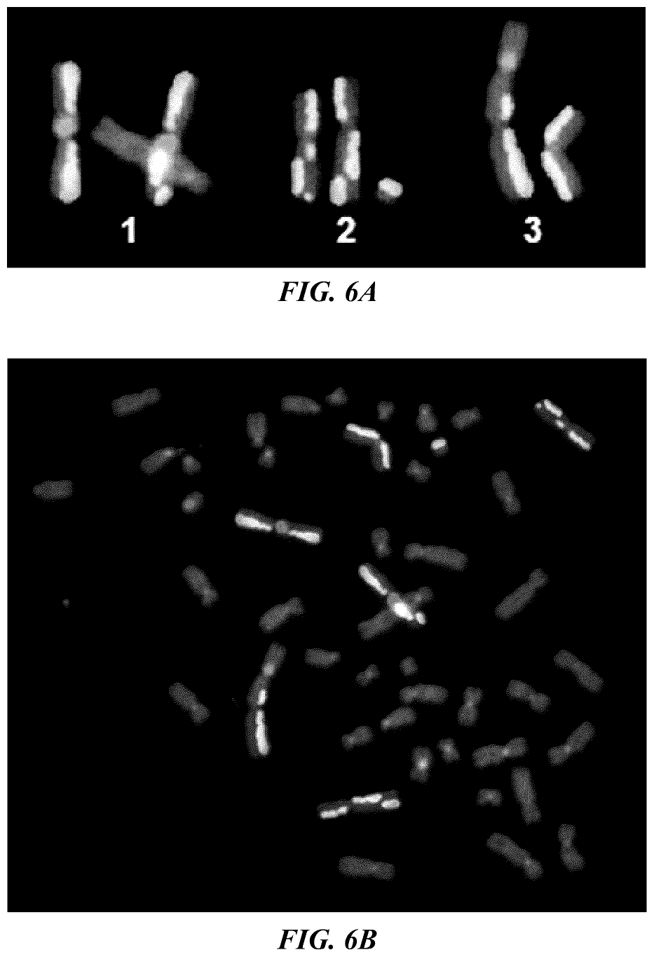

FIG. 6A-6B provide representative images of single color dGH paint labelling (shown in gray scale) Chromosomes 1, 2, and 3 in a rearranged cell from a metaphase spread of a radiation exposed blood-derived lymphocyte sample prepared for dGH analysis. dGH paints are dGH assays that include one or more dGH probes whose target DNA sequence or combined target DNA sequence(s) span a large section of a chromosome such as an arm, or virtually an entire or an entire chromosome. In the experiment provided in this Example, each chromosome was painted with a single dGH probe (i.e. a pool of oligonucleotides each individually labeled with the same fluorescent label). Images were acquired on an ASI scanning microscope system and were viewed using GenASIS cytogenetics software. The chromosomes from the selected metaphase were organized by the software into a karyogram (displays chromosomes in vertical orientation and organizes them into homolog pairs from original image of full metaphase spread) and the labelled Chromosome 1, Chromosomes 2 and Chromosome 3 homolog pairs were cropped and enlarged as shown in FIG. 6A, from an original entire metaphase spread image, shown in FIG. 6B. In this cell there are obvious rearrangements involving the painted chromosomes (Ch 1, 2 and 3), but confirming the presence of true structural variants versus sister chromatid exchange events is not possible without any reference for segmental order at the locations a signal switch is observed to the un-painted sister chromatid, nor is it possible to determine the genomic coordinates of the observed events on each chromosome.

### Example 2. Banded dGH for Chromosome 2 Using Fibroblast Cell Line

A human chromosome 2 dGH multi-color band pilot experiment was performed using the BJ-5ta normal human fibroblast cell line.

Experimental Description: A dGH reaction was performed using a standard dGH protocol by culturing a fibroblast cell line, BJ-5ta, in the presence of a nucleotide analog mixture for a single round of replication (a single S phase) and single-stranded sister chromatids were generated upon enzymatic digestion of labeled strands. The single-stranded sister chromatids were then contacted with the dGH probes disclosed herein below.

Referring to Table 1, nineteen dGH probes that bound to a single strand of chromosome 2 were labeled in an alternating color pattern with respect to their target DNA sequence on chromosome 2, with 5 different fluorophores. Each dGH probe was made up of a pool having the same number of oligos (27390 single-stranded oligonucleotides, labeled with the same fluorophore), except for the dGH probe that bound to a target DNA sequence near the terminal end of Chromosome 2, which had roughly 1.6× the number of oligos (44561 single stranded oligonucleotides) in its pool. Depending on the distribution of available unique sequences across Chromosome 2, the oligo pools were complementary to target DNA sequences that were spread across longer or shorter stretches of DNA, such that fluorescence analysis based on the hybridization pattern of the dGH probes resulted in a fluorescence “fingerprint pattern” of color bands unique to Chromosome 2. The target DNA sequences and resulting color bands forming the fluorescence pattern ranged in size from between 9 Mb and 21 Mb. See Table 1 for location in bp start to end on chromosome 2 for each target DNA sequence (band) for each labelled pool of oligonucleotides that make up a dGH probe, the total target DNA sequence size in bp of each labeled pool of oligos (i.e. dGH probe), the number of bound oligos that generated a band (i.e. the number of oligos per labelled pool (i.e. dGH probe), and the density distribution of fluorophores across the target region of DNA when these dGH probes are bound to their target DNA sequences. Also included in the table are the pseudocolor assignments for each fluorophore (i.e., band color for Watson strand). Some fluors are outside of the visible spectrum and/or have colors that are visually similar to one another in an overlay, so each color channel was assigned a pseudocolor that allowed for visualization of the bands as distinct from one another. The order of the colors in the table as well as the template strand assignment (Watson and Crick as they correspond to each sister chromatid) is delineated. The color assigned to the “Crick” sister chromatid is blue, reflecting the DAPI DNA stain color, since the dGH probes in Table 1 were directed to target DNA sequences on the Watson strand. The telomere, subtelomere, and centromeric regions are also DAPI blue in this experiment since dGH probes used in this experiment did not contain target DNA sequences in these regions (i.e., these regions are not labelled by a dGH probe). The band colors and strand assignment reflect the genomic coordinates of a normal metaphase chromosome 2 (prepared for dGH). For this experiment, the band sizes ranged from 9-21 million base pairs (MB). A few control probe spots (i.e., control dGH probes and their target DNA sequences) were included on both Chromosome 8 and Chromosome 1 for confirmation of resolution and hybridization quality. Please note the images included for all of the experiments involving this multi-color dGH analysis of virtually the entire chromosome 2, were converted to black and white, and the full color spectrum must be inferred using Table 1 and the order of the appearance of the bands. This experiment is referred to as a multi-color paint experiment because dGH probes bound to target DNA sequences on virtually an entire strand of chromosome 2 with short gaps between target DNA sequences (see start and end nucleotide numbers in Table 1) and with no target DNA sequences on the telomere, subtelomere, or centromere regions of chromosome 2.

The remainder of this page is intentionally left blank.

TABLE 1

| | | | | | | | average |
|---|---|---|---|---|---|---|---|
| Feature and | | | | | | | fluorescence |
| Colored Band | | | | Number | Band Color/ | Band color/ | density |
| Number | Start | End | Size | of oligos | DNA label | DNA label | (target size/ |
| (p-->q arm) | (bp) | (bp) | (kb) | per band | (Watson) | (Crick) | # of fluors) |
| Telomere p-arm | | | | | Blue | Blue | |
| Subtelomere p-arm | | | | | Blue | Blue | |
| 1 | 14497 | 9199710 | 9185213 | 27390 | Red | Blue | 1 fluor per 335 bp |
| 2 | 9199917 | 19417428 | 10217511 | 27390 | Green | Blue | 1 fluor per 373 bp |
| 3 | 19417468 | 29156419 | 9738951 | 27390 | Red | Blue | 1 fluor per 355 bp |
| 4 | 29157122 | 40996360 | 11839238 | 27390 | Green | Blue | 1 fluor per 432 bp |
| 5 | 40998055 | 52053266 | 11055211 | 27390 | Red | Blue | 1 fluor per 404 bp |
| 6 | 52054602 | 65033440 | 12978838 | 27390 | Green | Blue | 1 fluor per 473 bp |
| 7 | 65033522 | 75198573 | 10165051 | 27390 | Magenta | Blue | 1 fluor per 371 bp |
| 8 | 75198607 | 96577268 | 21378661 | 27390 | Yellow | Blue | 1 fluor per 780 bp |
| Centromere | | | | | | Blue | |
| 9 | 96577275 | 107055858 | 10478583 | 27390 | Magenta | Blue | 1 fluor per 382 bp |
| 10 | 107055871 | 120339051 | 13283180 | 27390 | Yellow | Blue | 1 fluor per 484 bp |
| 11 | 120339115 | 133399731 | 13060616 | 27390 | Magenta | Blue | 1 fluor per 476 bp |
| 12 | 133399786 | 146189594 | 12789808 | 27390 | Yellow | Blue | 1 fluor per 466 bp |
| 13 | 146189670 | 159967358 | 13777688 | 27390 | Green | Blue | 1 fluor per 503 bp |
| 14 | 159967656 | 173217068 | 13249412 | 27390 | Orange | Blue | 1 fluor per 484 bp |
| 15 | 173217075 | 187214405 | 13997330 | 27390 | Green | Blue | 1 fluor per 511 bp |
| 16 | 187214412 | 202327837 | 15113425 | 27390 | Orange | Blue | 1 fluor per 552 bp |
| 17 | 202327998 | 215789823 | 13461825 | 27390 | Green | Blue | 1 fluor per 491 bp |
| 18 | 215789917 | 225233519 | 9443602 | 27390 | Orange | Blue | 1 fluor per 344 bp |
| 19 | 225233538 | 241778486 | 16544836 | 44561 | Magenta | Blue | 1 fluor per 371 bp |
| Subtelomere q-arm | | | | | Blue | Blue | |
| Telomere q-arm | | | | | Blue | Blue | |

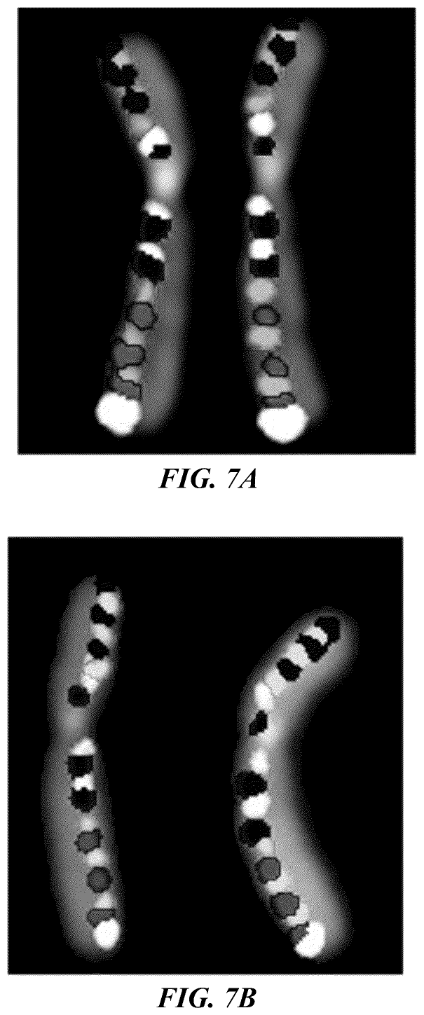

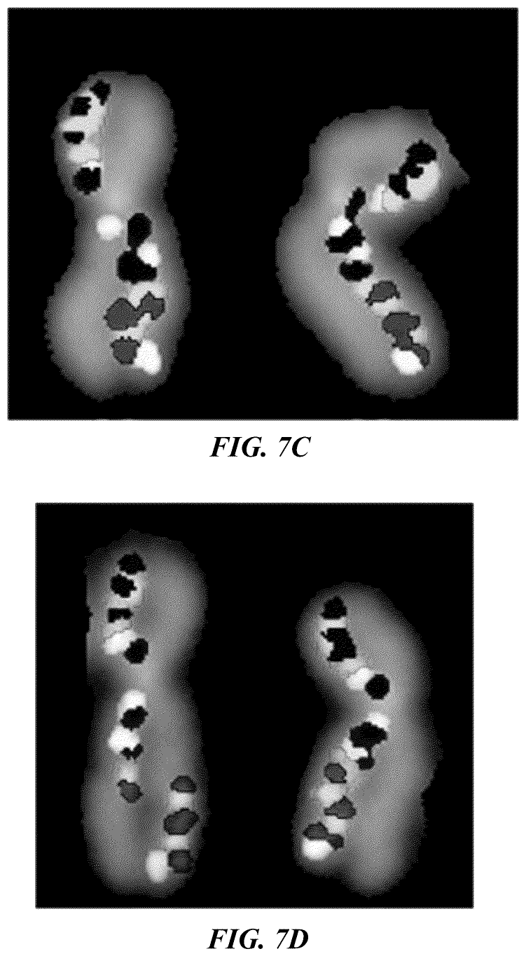

Images provided in FIG. 7A and FIG. 7B show chromosome 2 homolog pairs from two separate normal metaphase cells, which have no structural variation present (normal immortalized human fibroblast line BJ-5ta). Each of the two fluorescence patterns disclosed in FIG. 7A and the two fluorescence patterns disclosed in FIG. 7B, were based on fluorescence generated by the hybridization pattern of dGH probes along a single-stranded sister chromatid produced in a dGH reaction that included each of the chromosome 2 homolog pairs displayed in the figure. FIGS. 7A and 7B images (shown in gray scale) were acquired on an ASI scanning microscope system and were viewed using GenASIS cytogenetics software. The chromosomes from the metaphases selected were organized by the software into a karyogram (displays chromosomes in vertical orientation and organizes them into homolog pairs from original image of full metaphase spread) and the labeled Chromosomes 2 homolog pairs were cropped and enlarged from the original metaphase spread image.

In addition, 2 cells displaying abnormal signal patterns (from the same experiment using the same cell line) were imaged and analyzed. FIG. 7C and FIG. 7D images (shown in gray scale) show Chromosome 2 homolog pairs from 2 separate metaphase cells (normal immortalized human fibroblast line BJ-5ta) showing structural variation in one homolog resulting from sister chromatid exchange (the order of the colors in the fluorescent banding pattern is maintained, but the signals are present on the opposite sister chromatid). NOTE: where a single color paint is used, a telomere or sub-telomeric dGH probe is necessary for distinguishing between a large inversion (mis-repair) and a sister chromatid exchange (perfect repair) event. The classification of this type of event can be confounded using the single-color paint plus telomere/sub-telomere approach if there is an additional sister chromatid recombination event in the telomeric or sub-telomeric region. The embodiment demonstrated in this Example allows for both the detection and accurate classification of the structural rearrangement events. In FIG. 7C, the Chromosome 2 homolog on the left has an SCE with the breakpoint of the repair event bisecting band #13, and the homolog on the right is normal. In FIG. 7D, the homolog on the left has an SCE with the breakpoint occurring between bands #9 and #10, and the homolog on the right is normal.

### Example 3. Chromosome 2 Banded dGH Using Lymphocytes

This Example provides a chromosome 2 dGH multi-color band pilot experiment using blood-derived lymphocytes recently exposed to ionizing radiation for prostate cancer treatment.

Using the dGH assay described provided in Example 12, the dGH assay which utilizes 19 pools of labeled, single-stranded unique sequence oligonucleotides comprising between 10,000 and 50,000 that include 27390 or 44561 oligos per dGH probe. The dGH probes when used in a dGH reaction creating a fluorescent pattern of bands spanning 9 MB-17215 MB each, labeled in an alternating color pattern such that the order of the colors corresponds to the genomic coordinates of a normal metaphase chromosome 2. The dGH probes were used in a dGH reaction with single-stranded chromatids prepared from was run on a radiation exposed, blood-derived lymphocyte samples prepared for dGH.

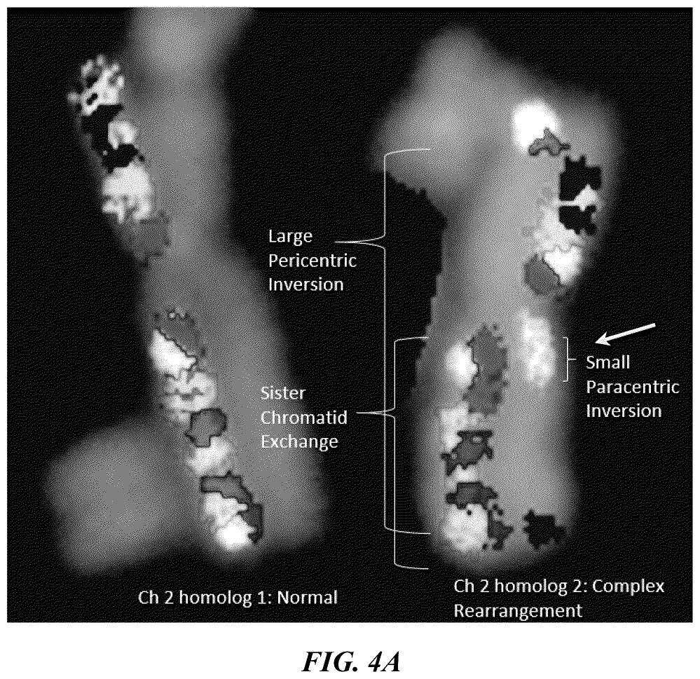

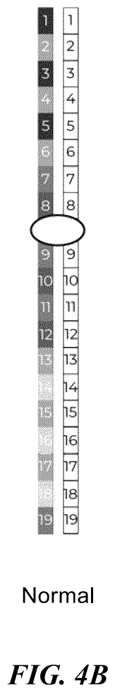

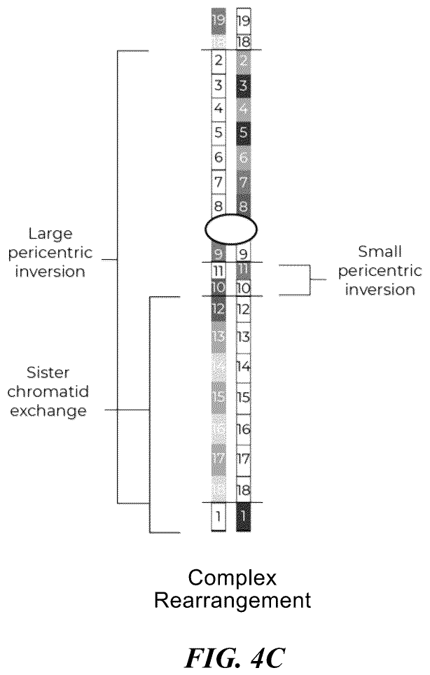

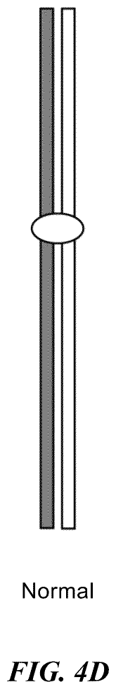

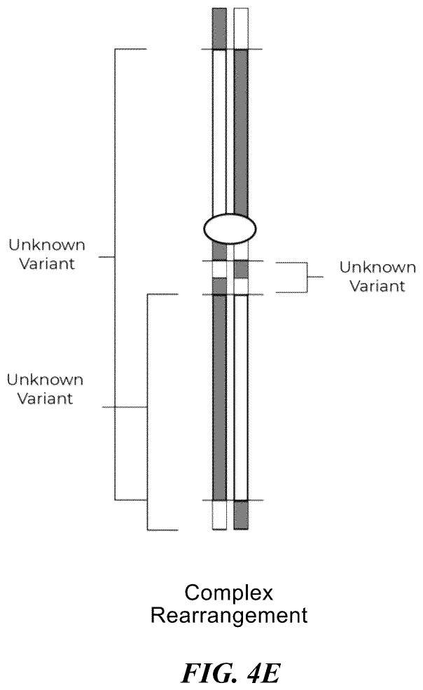

FIG. 4A provides fluorescence images (shown in gray scale) with overlayed multicolor banding of the dGH assay performed in this Example, for a chromosome 2 homolog pair from a metaphase cell with SVs identified that would otherwise be very difficult and likely impossible to characterize by current cytogenetic techniques. Comparison of the multi-color dGH banding pattern on the two homologs reveals complex structural variations in one of the sister chromatids compared to its homolog from the same cell. Analysis of fluorescence banding patterns revealed a large pericentric inversion present (potentially detectable by current cytogenetic techniques, but likely to be missed due to the nature of the band disruption taking place at the very distal ends of the chromosome), along with a smaller paracentric inversion (arrow in right side image of FIG. 4A pointing to dGH probe out of order on opposite sister chromatid from the majority of the labeled pools on the q-arm) near the centromere, and a larger sister chromatid exchange event in very close proximity to the smaller paracentric inversion. All of these can be described using the alternating colors of the banded dGH assay as a frame of reference. Rearrangements difficult to visualize in a color-combined overlay (shown in gray scale) can be confirmed by viewing signals on each separate color channel.

The diagrams provided in FIG. 4B-FIG. 4E illustrate how the complex rearrangement appears using the multi-color banded dGH paint used in this experiment compared to a monochrome dGH paint. FIG. 4B provides a diagram of a normal chromosome 2 showing target DNA sequences illustrated as gray scale bands (1-19) representing the chromosome 2 dGH paint with multi-color bands used in this experiment, and as actually observed in the left-side image in FIG. 4A. FIG. 4C provides a corresponding diagram of a chromosome 2 with complex structural rearrangements as labeled, and as actually observed in the right-side image FIG. 4A.

FIG. 4D shows a normal Chromosome 2, prepared for dGH, hybridized with monochrome Ch 2 dGH paint. FIG. 4E shows Chromosome 2 with complex structural rearrangements hybridized with monochrome Ch 2 dGH paint. The color map for individual dGH bands (1-19) shown in grayscale images is provided in FIG. 10A. As shown in the diagrams of FIG. 4D and FIG. 4E, if this cell had been labelled with a monochrome Ch 2 dGH paint, this chromosome would appear to have a small terminal SCE or inversion (p-arm), and a large inversion (q-arm), and the true classification of the structural rearrangements present would have been missed.

In more detail, the multi-color banded dGH image in the right side of FIG. 4A reveals that a large pericentric inversion is present, with one breakpoint occurring between bands 1 and 2 on 2p and the other bisecting band 18 on 2q. An additional smaller paracentric inversion is present near the centromere on 2q with the first breakpoint between bands 9 and 10, and the second break point between bands 10 and 11. A large sister chromatid exchange event between bands 9 and 11, sharing the same proximal break point with the small paracentric inversion is also present and can be verified with the order of the bands, which still appear in the correct numerical order, but are now on the opposite sister chromatid (left sister chromatid) from the primary paint (right sister chromatid). Without spectral detection and analysis of the colored bands providing the order of the segments, the rearrangements cannot be identified or described in coordinates. In fact, using the schematic in FIG. 4E in relation to FIG. 4D, for visual reference, the chromosome appears to have a small terminal SCE or inversion (p-arm), and a large inversion (q-arm), and the true classification of the structural rearrangements present would have been mis-identified.

### Example 4. Detection of an SCE Using Banded dGH

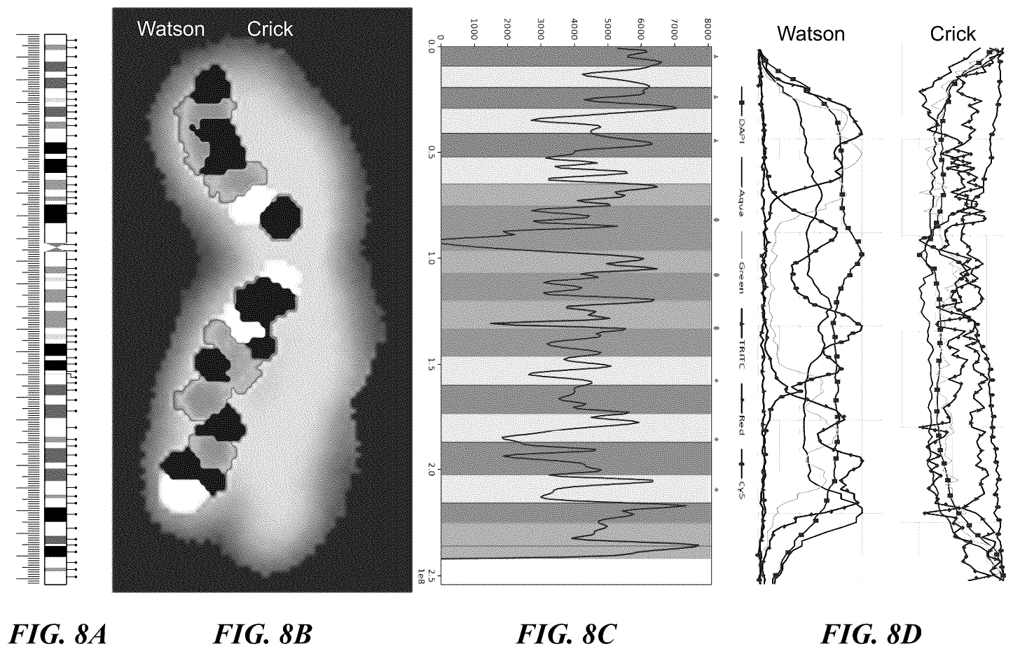

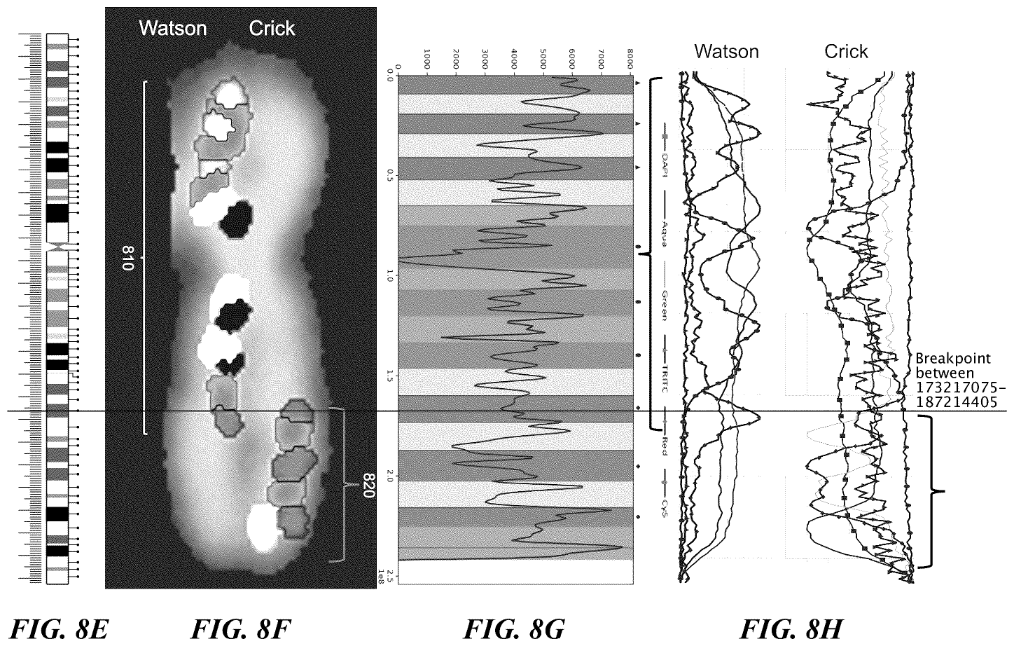

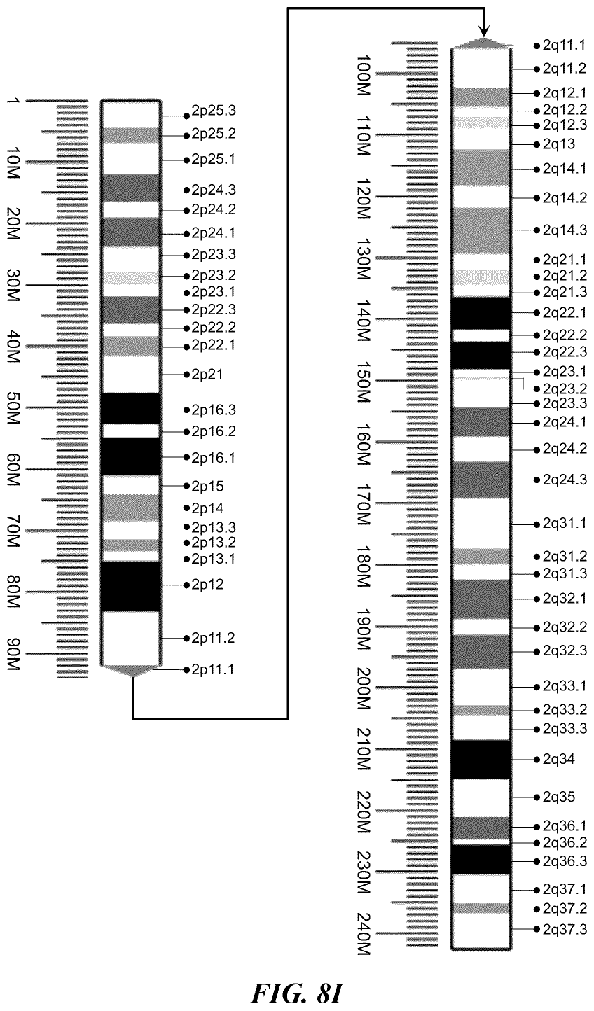

Using the dGH assay, cell line, and imaging method from Example 2, fluorescent images and spectral intensity measurements along each sister chromatid were analyzed across a normal Chromosome 2 and a test chromosome 2 containing an SCE repair event. Fluorescent patterns and spectral profiles of the normal chromosome 2 and from the test chromosome 2 were generated by using fluorescence microscopy and imaging analysis software to analyze images and spectral measurements including fluorescent wavelength and intensity measurements across each of the sister chromatids. FIG. 8A-FIG. 8D relate to the normal chromosome 2 sample and FIG. 8E-8H relate to the test chromosome 2 sample in which an SCE is present (all images shown in gray scale). The figures in these correlated sets of 4 figures (FIG. 8A-FIG. 8D and FIG. 8E-FIG. 8H) show the hybridization pattern/image overlay (FIG. 8B and FIG. 8F, probe distribution (FIG. 8C and FIG. 8G), and fluorescent wavelength intensities (FIG. 8D and FIG. 8H), respectively. FIG. 8A and FIG. 8E show an ideogram of Chromosome 2 for genomic context, which can be seen in greater detail in the enlarged image, FIG. 8I. FIG. 8B and FIG. 8F show an image overlay of the hybridization pattern of imaged dGH probes from analysis of fluorescent signals, overlaid on background fluorescence of chromosome 2 and aligned with the ideogram of the corresponding FIG. 8A and FIG. 8E, respectively. 810 of FIG. 8F shows the hybridization pattern of dGH probes on one chromatid of chromosome 2, and 820 of FIG. 8F shows the exchanged portion of the chromatid originally hybridized with dGH probes, now on the sister chromatid. For the sake of clarity, the bands shown in FIG. 8A, FIG. 8E, and FIG. 8I (enlarged image) are G bands produced using Giemsa staining, not by banded dGH analysis. FIG. 8C and FIG. 8G show the oligonucleotide distribution (y axis) of the pools of oligoes that made up the dGH probes plotted along the length of chromosome 2 (x axis) with dGH bands as shown. FIG. 8D shows the fluorescent wavelength intensities (y axis) plotted along the length of the chromosome. The signal intensity profile on each color channel for each sister chromatid is shown by the 6 overlapping line graphs, thus providing a spectral profile of normal chromosome 2. The fluorescent banding pattern determined by the measurements in 8 D, as described, is shown as vertical bands along the chromosome in FIG. 8C. The sister chromatids were designated as “Watson” and “Crick”, and color channels were measured for both sister chromatids. On sister chromatid Crick, signal intensity displayed in FIG. 8D represents background noise on each channel, with the actual signal intensity peaks visible on Watson since the dGH probes used bound to the Watson strand.

### Example 5. Using Ladders as Internal Controls

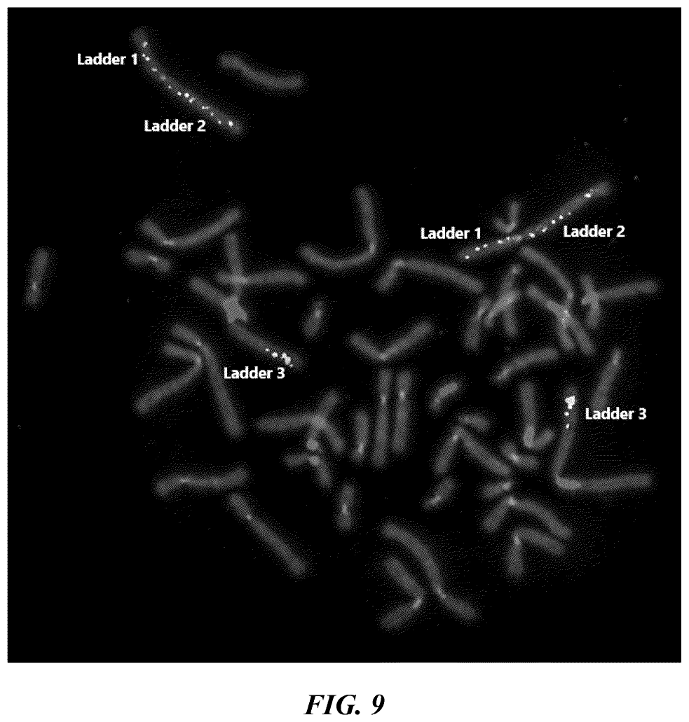

Ladder images—Introduction: The chromosome condensation (compact vs long) in metaphase spread preparations varies between cells and between cell preparations. This material variability can be accounted for in an assessment before determining the resolution of SV detection by dGH assays. For example, in longer, more stretched configurations of chromatin, hybridization signals from dGH probes spaced close together can be resolved as separate signals, and in more compact and condensed chromatin, hybridization signals from dGH probes spaced closely together will appear as a single merged signal. In the metaphase spread as shown in FIG. 9 (shown in gray scale), three separate dGH probe ladders (also referred to as ladder assays) were hybridized to the chromosomes. One ladder assay measures limit of detection with respect to the number of oligos contributing to each signal, spaced roughly 20 Mbmb apart on the p-arm of Chromosome 2 (labelled Ladder 1 in the image). The number of oligos per pool of a dGH probe can range from ranges in number of oligonucleotides from as little as 10 oligonucleotides to over 10\hat{ }6. A second ladder assay (Chromosome 2q) assesses the target size a fixed amount of oligos can be spread out over, also spaced about 20 Mb apart, and also measures limit of detection (labelled Ladder 2 in the image). A third ladder assay (seen below shown in FIG. 9 hybridized to Chromosome 1q), has dGH probes spaced close together as well as farther apart, allowing for an assessment of the resolvability two spots in close proximity in any given metaphase spread (labelled Ladder 3 in the image FIG. 9). These ladders are designed against the opposite DNA strand from the banded paints and can be used as an internal control for the assay resolution in each spread.

### Example 6. Marker Oligonucleotides and dGH Assay

An assay including marker oligonucleotide hybridization of fragile-site associated Alu repeats in one color and multi-color banded dGH paints in other colors can be run on a metaphase sample prepared for dGH. Alu repeats (which have been characterized and mapped in the reference genome) can be displayed and detected as a unique banding pattern strongly associated with known fragile sites and regions known to be important for gene regulation such that the proximity of observed known or de novo rearrangements can be compared to known fragile regions. Structural variants present in rearranged chromosomes as visualized by the assay can be used to correlate phenotype to genotype as they relate to known high-risk regions of the genome.

### Example 7. Targeted Banded dGH

Multi-colored banded paints can be combined with two specific color bands assigned to regions bracketing a target of interest, and run on sample metaphases prepared for dGH. In the same field of view, the two colors bracketing the target of interest can be displayed in the interphase cells (nuclei) as an intercellular targeted dGH probe “break-apart” assay showing specific regional activity separate from the rest of the chromosome paint via selective analysis of specific color channels, allowing for the analysis of cells in the G1, S, and G2 phases of the cell cycle alongside the cells that have passed all the cellular checkpoints and have successfully entered metaphase. There are frequently more interphase nuclei present in a sample than there are metaphases on a slide preparation, and any nuclei present will be hybridized with dGH probe at the same time as the metaphase spreads. Using spectral profile determination and analysis, several types of data, in layers, can be provided by a single assay when coupled with specific imaging methods to visualize regions of the genome separately and as they relate to one another in a sample containing both metaphase cells and interphase cells.

### Example 8. Detection of ecDNA

A cancer cell line with visible large extrachromosomal DNA (ecDNAs) of unknown origin can be hybridized with dGH whole chromosome paints with unique colors for each human chromosome. dGH whole chromosome paints are dGH assays that include one or more dGH probes whose target DNA sequence or combined target DNA sequence(s) span virtually an entire chromosome. The chromosomal DNA amplified and contained in the ecDNAs will contain the same color or colors of signal as the chromosome(s) of origin. Once identified, the specific chromosome(s) known to contain genetic material also present in the ecDNAs can be run in a successive hybridization with the banded paint or paints corresponding to the previously identified chromosomes of origin. The region or DNA coordinates can be identified with spectral profile determination as the labeled ecDNA will correspond to a specific band or bands color in the banded chromosome. Coordinates can be further refined with specific targeted dGH probes for the identified region of origin, which will appear on both the ecDNA and the corresponding chromosomes and can be used to track and describe potentially deleterious changes to the genome.

### Example 9. Whole Genome dGH Banding

Using a dGH assay design similar to that used for chromosome 2 in Example 2, a set of dGH probes were designed to generate a multi-color dGH banding pattern for every chromosome of the human genome, except the Y chromosome for which only 1 dGH probe was designed. Table 3 provides the number of bands that were in the assay design for each chromosome in the human genome of both a haploid cell (1N) and a diploid cell (2N). The probes were labeled with 1 of 5 difference fluorophores and target DNA sequences were selected such that an alternating color pattern would be generated for each chromosome except the Y chromosome. Each dGH probe was made up of a pool of from about 500 to 10,000 oligonucleotides. Depending on the distribution of available unique sequences across each chromosome, the oligo pools were complementary to target DNA sequences that were spread across longer or shorter stretches of DNA.

A dGH reaction and imaging method was performed according to Example 2. Fluorescent images were generated for each sister chromatid for each chromosome of an entire genome of a human cell in a metaphase spread. As shown in FIG. 11 (shown in gray scale), in the karyogram images and matched ideograms to the left, and as observed in other analysis using this whole genome dGH assay, for each chromosome the expected banding pattern was observed. It should be noted that in some images depending on overlap among chromosomes or sister chromatids or the genomic structural variations and sister chromatid exchanges present, the patterns can be overlapped or varied. In combining the channels into a single overlay, some of the bands can be “masked” by neighboring bands. However, additional images can be obtained for any specific color channel separate from the combined image to observe any masked bands. In summary, the hybridization pattern of the dGH probes resulted in the expected unique banding pattern for each chromosome, with bands that ranged from about 2.5 Mb to 21 Mb in size.

TABLE 3
Whole Genome dGH Banding

| Human Chromosome | Number of | Number of |
|---|---|---|
| Number | bands (1N) | Bands (2N) |
| Ch 1 | 20 | 40 |
| Ch 2 | 20 | 40 |
| Ch 3 | 17 | 34 |
| Ch 4 | 13 | 26 |
| Ch 5 | 15 | 30 |
| Ch 6 | 13 | 26 |
| Ch 7 | 13 | 26 |
| Ch 8 | 12 | 24 |
| Ch 9 | 10 | 20 |
| Chr 10 | 13 | 26 |
| Ch 11 | 11 | 22 |
| Ch 12 | 11 | 22 |
| Ch 13 | 7 | 14 |
| Ch 14 | 7 | 14 |
| Ch 15 | 7 | 14 |
| Ch 16 | 8 | 16 |
| Ch 17 | 8 | 16 |
| Ch 18 | 8 | 16 |
| Ch 19 | 5 | 10 |
| Ch 20 | 7 | 14 |
| Ch 21 | 3 | 6 |
| Ch 22 | 3 | 6 |
| Ch X | 9 | 18 |
| Ch Y | 1 | 2 |
| Total | | 482 |

### Example 10. Car-T Cell Sorting and dGH Analysis

This prophetic Example provides a protocol for sorting a population of T cells into a metaphase-enriched T cell population, which is then contacted with probes and analyzed. The protocol utilizes a pooled population of engineered CAR-T cells: Cell sorting by BrdU+Hoechst and T cell staining panel (96 sample array):
1. Live T cells are stimulated to proliferate in vitro for a specified period of time (dependent on sample)
2. Live T cells are incubated with a BrdU/C analog for one cell cycle
3. Samples are stained for flow sorting using a memory vs effector T cell marker panel and Hoechst (to stain for cells in metaphase)
4. Cells are sorted using fluorescence activated cell sorting (FACS)
5. Sorted cells are fixed using a dGH protocol and spotted onto slides for dGH analysis (in this non-limiting prophetic example, many samples are spotted per slide and 1-3 assays are performed per slide)
   Note that this is an example of protocol for analyzing CAR T cells (see FIG. 13, which in illustrative embodiments is a CLIA-certified assay, where multiple samples or patients are enriched for sub-populations in metaphase, then hybridized with a single dGH probe (sometimes referred to as a panel of probes) that is made up of a pool of oligonucleotides that bind their complementary DNA sequence within a target DNA sequence, or a set of dGH probes (i.e. one assay).

### Cell Preparation (Steps 1 and 2)

Note: FACS buffer (0.5% BSA in PBS) should be kept ice-cold.
1. After the CAR-T cells are incubated with BrdU/C analog for 1 cell cycle, colcemid is added and incubated for 4 hours.
2. At the end of colcemid incubation, cells are transferred from the cell culture plate to 15 ml conical tube.
3. Cells are centrifuged at 1500 rpm, 5 min at 4° C.
4. Supernatant is removed and discarded.
5. Cells are washed once with 5 ml FACS buffer.
6. Supernatant is removed and discarded.
7. Cells are resuspended in 100 μl FACS buffer.
8. Cells are transferred to round-bottom 96-well plate.

### Surface Antibody Panel and Hoechst Staining for Distinguishing Naïve, Memory, and Effector T Cell Populations in Metaphase (Step 3)

Panel (based on Saxena A, Dagur P K, Biancotto A. Multiparametric flow cytometry analysis of naïve, memory, and effector T cells. Methods Mol Biol. 2019; 2032:129-140. doi: 10.1007/978-1-4939-9650-6_8 and Mousset C M, Hobo W, Woestenenk R, Preijers F, Dolstra H, van der Waart A B. Comprehensive Phenotyping of T Cells Using Flow Cytometry. Cytometry A. 2019 June; 95(6):647-654. doi: 10.1002/cyto.a.23724):

Note: Fluorophores are based on FACS sorter capabilities.
1. CD3
2. CD4 (if pan-T population)
3. CD8 (if pan-T population)
4. CD45RA
5. CD197 (CCR7 receptor)
6. Live/Dead stain

### Procedure:

Note: FACS buffer (0.5% BSA in PBS) and Nuclei Staining Buffer should be kept ice-cold.

Hoechst staining steps 11 and 12 are based on Sanders A D, et al. Nat Protoc. 2017 June; 12(6):1151-1176. doi: 10.1038/nprot.2017.029.
1. Fc block is added at 1:50 dilution. Incubate on ice 20 min.
2. Centrifuge at 1500 rpm, 5 min at 4° C.
3. Remove and discard supernatant by turning plate upside down into a sink.
4. Resuspend in 100 μl FACS buffer containing antibody cocktail (above).
5. Incubate 30 minutes at 4° C. in dark.
6. Centrifuge at 1500 rpm, 5 min at 4° C.
7. Remove and discard supernatant.
8. Add 200 μl FACS buffer to each well and centrifuge at 1500 rpm, 5 min at 4° C.
9. Remove and discard supernatant.
10. Repeat steps 8 and 9 an additional 2 times for a total of 3 washes.
11. Resuspend cells in 200 μl Nuclei Staining Buffer (salt buffer containing BSA, NP40, and Hoechst 33258).
12. Incubate on ice in dark, 15 minutes.
13. Filter samples through a cell strainer into a 5 ml round-bottom polystyrene Falcon tube.

### Cell Sorting (Step 4)

The specifics of this procedure depend on the type of sorting system chosen. The above protocol is compatible with FACS-based systems. Other sorting approaches may require different sample preparations. Below is an example of how different subpopulations can be identified using the panel plus Hoechst staining. First, dead cells and debris will be excluded based on forward/side scatter (FSC/SSC) and live/dead staining.

Next, cells will be identified as naïve, effector, or memory subpopulations:
Näive: CD3⁺CD45RA^{high/+}
Effector: CD3⁺CD45RA^{low/-}CD197⁻
Memory: CD3⁺ CD45RA^{low/-}CD197⁺

Within each T cell subpopulation gate, metaphase cells will be sorted based on Hoechst staining.

In the end, 3 distinct populations will be isolated for each sample:
1. Näive T cells in metaphase
2. Effector T cells in metaphase
3. Memory T cells in metaphase

### Sample Fixation and dGH Analysis (Step 5)

After cells are sorted, they are fixed with a 3:1 methanol:acetic acid solution, and optionally exposed to a 75 mM KCl solution before fixing, and ready to drop onto slides using an array spotting methodology and then further processed for dGH analysis by degrading one of the single-stranded sister chromatids, hybridizing dGH probe(s), and analyzing fluorescence signals generated by the probes. Multiple samples/patients can be dropped (e.g. immobilized) onto a single slide (1402) for example in a two-dimensional spatial arrangement of partitions such as an array of addressable positions (e.g., 1404) on a solid support (FIG. 14), and assayed with 1-96 assays, with each assay being a set of dGH probes and each dGH probe, sometimes referred to as a panel of probes, made up of a pool of oligonucleotides. The number of assays performed depends on the target/hybridization strategy employed.

### Example 11. iPSC Cell Sorting and dGH Analysis

This prophetic Example provides a protocol for sorting a population of iPSCs into a metaphase-enriched iPSC population, that is then contacted with dGH probes and analyzed. The protocol utilizes a population of iPSCs generated from a single patient with an idiopathic neurodegenerative disease: Cell sorting by BrdU+Hoechst (96 assay array).
1. Live iPSC are incubated with DNA analog (e.g. BrdU) for one cell cycle
2. Sample is stained for flow sorting using BrdU/Hoechst method (to stain for cells in metaphase)
3. Cells are sorted using fluorescence activated cell sorting (FACS)
4. Sorted cells are fixed using dGH protocol and spotted onto slides for dGH analysis (1 sample, 96 different assays on a single slide)

This is an example of a discovery/screening panel (see FIG. 13), where a single sample is enriched for metaphase cells, spotted onto a slide, then probed with 96 different panels of probes (i.e. assays). For these types of screening methods, there would be a 96-probe assay designed against known disease targets that can be screened for in each spot on the slide. In the case listed above, the targeted probes would be designed against genes that are known to be involved in neurodegenerative disease.

These cells are prepared using the Hoechst staining method described in Example 10 (no surface marker antibody staining is required for this assay). Slides will then be spotted with metaphase spreads of a known number, then assayed for 96 different targets.

Other technologies are possible using flow cytometry and dGH:
1. dGH-Flow using a combination of dGH probes and antibody panels. Can be used on traditional flow cytometers, automated cell sorters, an Amnis ImageStream, or other similar equipment;
2. Cell and chromosome sorting using dGH probes; In such methods panels of probes that bind single-stranded chromatids, as provided herein, can be used to label chromosomes different colors. Such labeled chromosomes can be used to sort the chromosomes or cells containing the chromosomes using a cell sorting method. Such sorted cells or chromosomes can be spotted on an array and/or can be used in methods provided herein, such as chromosome imaging methods.

### Example 12. Chromosome Sorting and dGH Analysis

This prophetic Example provides a protocol for sorting and isolating specific chromosomes (chromosome 8 in this example) from metaphase cells using a DNA specific stain, and then contacting and analyzing isolated chromosomes using dGH probes, and fluorescently labeled dGH probes to generate a bivariate flow-karyotype (chromosome sorting using dGH probes).

### Sample Preparation, Chromosome Isolation, and Hybridization:

1. Block cells at metaphase using 0.1 μg/ml colchicine for an amount of time optimized to maximize the percentage of mitotic cells. Cells can be exposed to colchicine for a time ranging from 30-60 minutes, or up to 8 hours, depending on cell type.
2. Pellet by centrifugation and resuspend cells in hypotonic KCl buffer, ranging from 60 to 85 mM KCl, for 10 mins at room temperature to lyse the cells.
3. Pellet by centrifugation and resuspend cells in chromosome isolation buffer (CIB) containing a stabilizing agent or agents such as divalent cations (magnesium ions) and cationic polyamines (spermine, spermidine) to generate good chromosome preparation. For example, the CIB can comprise the following formula: 15 mM Tris (hydroxymethyl)aminomethane, 2 mM Na2EDTA, 0.5 mM spermine, 80 mM KCL, 15 mM mercaptoethanol and 1% (v/v) Triton-X.
4. Vortex for 20 seconds.
5. Spin the chromosome suspension at 200 g for 2 mins and filter the supernatant through 20 m mesh filter into a 1.7 mL Eppendorf tube.
6. Preliminary probe hybridization: Resuspend in Kromatid FISH Hybridization Buffer (Kromatid Inc., Longmont, CO) pre-mixed with Chr8 dGH probe(s) that targets a target DNA sequence that encompasses virtually the entire chromosome 8, which can also be referred to as a type of chromosome paint, and includes an ATTO-550 fluorescent label.
7. Incubate at 75° C. for 3 min and transfer immediately to another heat block set at 37° C. for overnight incubation.
8. Perform 3 successive washes in 2×SSC heated to 43° C., each time adding 1 mL to the tube and then pellet hybridized chromosomes by spin at 200 g for 2 min.
9. Stain chromosomes for 2 hr with 5 μg/mL Hoechst 33258

### Flow Sorting of Chromosome 8:

1. Analyze the stained chromosome suspension on a MoFlo™, FACS Aria™, BD Influx™ or equivalent flow sorting instrument. Tune the first laser to emit multi-line UV (330-360 nm) to excite Hoechst 33258 and the second laser to emit light at 555 nm, to excite the two separate fluorophores.
2. Trigger the signal based on Hoechst 33258 fluorescence and collect Forward Scatter (FSC) using a 351/10 nm band pass filter, and appropriate filters for triggering the Atto550
3. Configure the instrument for high-speed sorting.
4. Set up the sort decision by creating sort gates on the selected chromosome clusters using a two colored sort/binning strategy (e.g. on a bivariate flow karyogram setting) and isolate a target number of chromosomes directly into tube or onto a microscope slide for further analysis.

Analyze the sorted chromosomes (e.g. Similar to the flow karyotypes, the chromomycin A3 fluorescence could be replaced by the Atto550 Chromosome 8 dGH paint fluorescence), such that only chromosomes containing Chr8 specific material would be represented in the karyotype.

### Example 13. dGH Arrays

This prophetic Example provides a protocol for the two-dimensional arrangement of cells and/or chromosomes in partitions on arrays for detection and subsequent analysis. As disclosed elsewhere herein, such arraying method in some illustrative embodiments can utilize sorted cells or sorted chromosomes/chromatids using any of the cell-sorting or chromosome-sorting methods provided herein.

Note: Cells should be dispensed in an environment with 55% ambient humidity.
1. Process: Specify the layout of cells to be arrayed.
   a. Number and spacing of wells
   b. Number of cells per well
   c. For multiple samples—which sample is spotted in each well
2. Cell samples should be fixed in a 3:1 methanol:acetic acid fixative solution. Resuspend cells by agitation before loading into the instrument (e.g. cellenONE® X1).
3. Load microscope slide and cell sample(s) and/or chromosome sample(s) into the instrument and activate the instrument to dispense the cells or chromosomes/chromatids onto the microscope slide.
   a. cellenONE® X1 or comparable instrument capable of single cell, contactless dispensing.
   i. Other methods of single cell dispensing include acoustic droplet dispensing, inkjet bioprinting, and image-based single cell printing.
4. Allow the slide to dry to immobilize the cells or chromosomes/chromatids in a two-dimensional spatial arrangement of partitions on a solid support (FIG. 14). For example, cells or chromosomes/chromatids from each sample of a plurality of samples can be loaded onto the same addressable array location within each partition (FIG. 14).
5. Proceed with standard process, such as a dGH analysis, after cells or chromosomes/chromatids are dropped onto the slide for example in a predefined placement of individual samples across the slide.

### Example 14. Fluorescence-Based Cell Sorting and dGH Analysis

This example provides a successful demonstration of fluorescence-sorting of a population of GM12753 B-lymphocyte cells (“GM cells”) immortalized with Epstein Barr virus from (Coriell Institute, International HapMap Project, CEPH/UTAH Pedigree 1447) into a metaphase-enriched hemi-substituted G2/M cell population.

### Methods

The protocol utilizes dividing GM cells with a doubling time of 17 hours.

### Sample Preparation:

1. Two vials of 5 million live GM cells were thawed into two separate T-25 cell culture flasks in 5 mLs prewarmed media and allowed to proliferate in vitro for 24 hours.

2. A BrdU/C analog mixture was added to one of the flasks, and both flasks were incubated for an additional 17 hours with colcemide added for the final 4 hours, and collected into separate 15 mL conical tubes containing either the BrdU/C(−) sample or the BrdU/C(+) sample, and washed 2× with PBS via centrifugation (1000 RPM for 10 min).

3. The cell pellets were resuspended into Nuclei staining buffer A (NSB A), containing the following components (final concentrations): 150 mM NaCl, 50 mM Tris-HCl, pH 7.4, 1 mM EDTA, 1% Triton X-100, 0.1% sodium dodecylsulfate (SDS), 10 μg/ml Hoechst 33258, and 10 μg/ml propidium iodide (PI), and stained according to Sanders A D, et al. Nat Protoc. 2017 June; 12(6):1151-1176. doi: 10.1038/nprot.2017.029, which is herein incorporated by reference in its entirety).

4. Cells in NSB A combined with Hoechst 33258 were then filtered through a 40 um filter and placed back on ice prior to performing fluorescence activated cell sorting (FACS).

### FACS Sorting:

FACS sorting was performed on a MoFlo XDP70 FACs instrument (Beckman-Coulter) at the Flow Cytometry Shared Resource Lab at the CU Cancer Center, Anschutz Medical Campus.

1. The BrdU/C(−) sample was used to calibrate the FACS machine. ˜20 k cells were run on the sorter to set gates on the populations of interest. Sort precision was set to single-cell mode with a 1 drop envelope.

2. Using the UV laser in linear scale, the Hoechst+ population was placed on the right-hand side of the plot, and a gate was drawn on this population to calculate the mean Hoechst fluorescence of the BrdU/C(−) sample.

3. The BrdU/C+ sample was loaded, and ˜20 k cells were run on the sorter to visualize the divided population, and to draw the sorting gate.

### dGH Processing

A control sample of non-sorted, BrdU/C(+)-treated GM cells were processed for dGH analysis using a single-color chromosome 1, whole chromosome dGH probe (i.e. chromosome 1 dGH paint) (similar to Example 1) and stained with DAPI DNA stain using standard conditions. For the dGH processing, cells from the initial non-sorted sample were cultured in the presence of BrdU/C(+) analog for 17 hours and treated in colcemid for the final 4 hours. The cells then were subjected to a dGH harvest procedure that included treatment with a 75 mM KCl hypotonic solution followed by fixation with a 3:1 methanol:acetic acid solution before the cells were used to prepare metaphase spreads that were contacted with the chromosome 1 dGH paint. Fluorescent microscopic images were acquired using an ASI scanning microscope system and were viewed using GenASIS cytogenetics software.

### Results

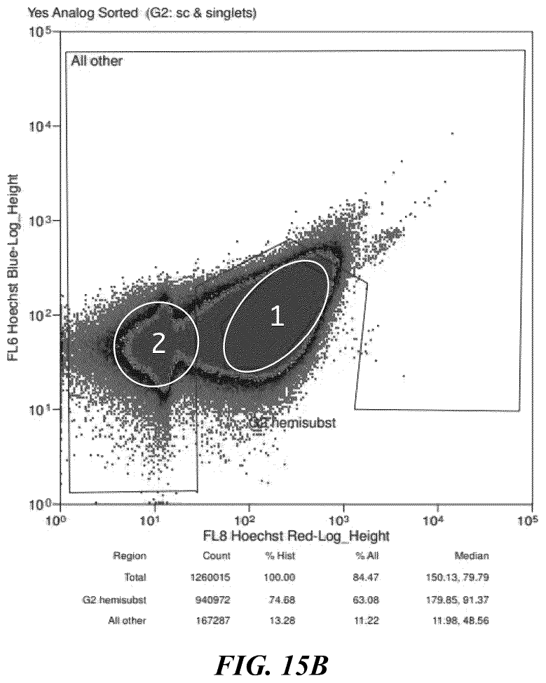

dGH and DAPI analysis alongside a distribution plot of the two cell populations supports the effectiveness of the fluorescence-based cell sorting method used in this Example to sort a dGH-processed cell population into a desired metaphase-enriched cell population with hemi-substituted chromatids that could be used for dGH analysis. Cells in G2/M which had taken up BrdU/C for a full S-phase should show ˜½ the Hoechst fluorescence of the BrdU/C (−) sample G2/M peak after cell sorting. Analysis of a cell cycle histogram of the cell population that was treated with BrdU/C and colcemid provided a G2/M cell population (FIG. 15A) that was gated for further analysis. Since the majority of the sample was in G2/M the gates of the sorter were set to capture the desired, left-shifted population, shown in FIG. 15B sorting region “1”. It is believed that this region contains the desired, hemi-substituted G2/M population of interest. The non-desired, double substituted cells in G2/M, showed up as even further shifted to the left on the scatter plot (See FIG. 15B, sorting region “2”). Cells appearing outside region “1” and to the right on the scatter plot were assumed to be non-desired, partially substituted or non-substituted cells in G2/M. A sorting desired region of interest (ROI) was drawn around the desired hemi-substituted G2/M population of interest (region 1 in FIG. 15B). And a non-desired ROI was drawn around everything else on the scatterplot, and sorting was run on the remainder of the BrdU/C(+) sample. In total, a desired cell population of 757,000 BrdU/C hemi-substituted cells in G2/M cells (i.e. metaphase-enriched, hemi-substituted cell population) were collected from the sample, as well as 126,000 non-desired cells (i.e. double-substituted combined with partial-substituted G2/M cells). Thus, although the majority of the cells in the sample were the desired cell population, a significant percent were not, underscoring the need for a method to further enrich metaphase cells for dGH processing.

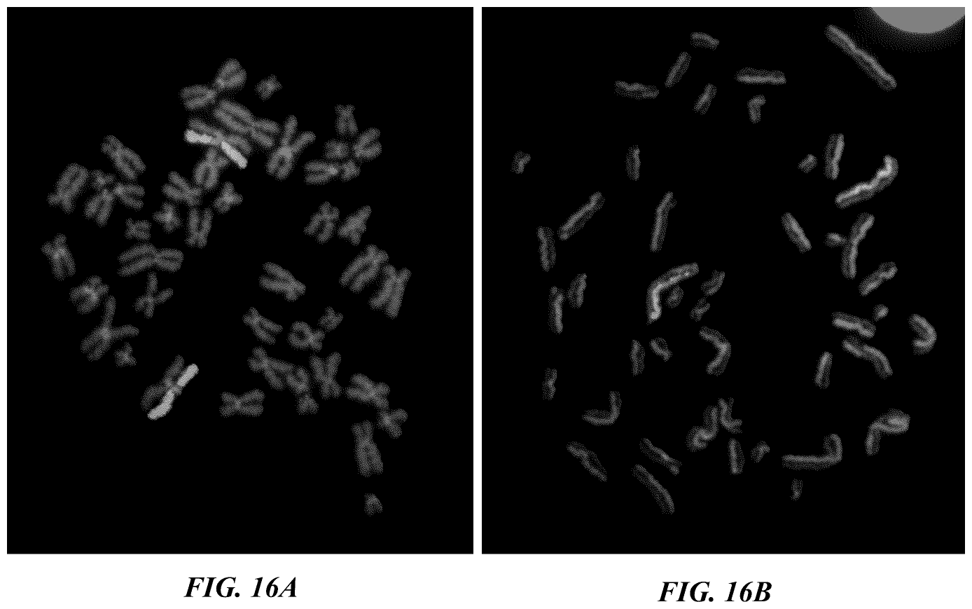

We next analyzed a control population of GM cells that were subjected to dGH processing to confirm that a majority of cells were metaphase-enriched, hemi-substituted cells, but also to confirm that a significant percentage of the cells were not. FIG. 16A is a fluorescence image of the DAPI stain and chromosome 1 dGH analysis of the control BrdU/C (+), hemi-substituted GM cell population before enrichment by fluorescence-based sorting. By analyzing the mitotic spreads of this control cell population the distribution of desired, hemi-substituted vs undesired double substituted cells and their relative distributions in the population are apparent and confirms that the majority of cells in the initial population were metaphase-enriched, hemi-substituted cells. However, the results also confirmed that a significant number of cells in the initial cell population without fluorescence sorting were not. More specifically, many of the metaphase spreads analyzed provided a correct and expected dGH paint pattern on one sister chromatid of chromosome 1 and even DAPI staining across chromosomes (FIG. 16A), indicative of metaphase-enriched, hemi-substituted cells. However, some of the chromosome spreads produced uneven paint hybridization, and a harlequin pattern with the DAPI staining (FIG. 16B), as expected for undesirable cells. Thus, the control GM cell population after dGH processing but without fluorescence cell sorting, was a mixed population of metaphase-enriched, hemi-substituted cells, that included a significant percentage of non-desired cells as well.

Thus, this example shows that with standard dGH processing, many cells remain in the cell population that are not metaphase-enriched, hemi-substituted cells, which are the cells most effectively analyzed using dGH analysis. Thus, a method that can further enrich a cell population that has undergone standard dGH processing, for these desired cells is needed. The fluorescence-based cell sorting method used in this Example provides a method for obtaining a further enriched subpopulation of metaphase-enriched, hemi-substituted cells.

### Example 15. Sample Array and dGH Analysis

This example provides a successful demonstration of dGH detection of a target chromosome on dGH processed cells spotted in a two-dimensional, regularly spaced arrangement array configuration on a support matrix. GM12753 B-lymphocyte cells (“GM cells”) immortalized with Epstein Barr virus (from Coriell Institute, International HapMap Project, CEPH/UTAH Pedigree 1447) were divided into four separate treatments (four samples; samples 1-4) for the timing of BrdU/C exposure (each sample exposed for a specific time period) and incorporation.

### Methods

### Sample Preparation:

1. GM cells were distributed as four samples (samples 1, 2, 3, and 4) and were allowed to proliferate in vitro for 24 hours. A BrdU/C analog mixture was added to the GM cells and the cells were incubated for an additional 16 hours (sample 1), 17 hours (sample 2), 18 hours (sample 3), and 20 hours (sample 4). Colcemid™ was added to each sample for the last 4 hours of the respective incubation time. The cells from each sample were then collected into separate 15 mL conical tubes and washed 2× with PBS via centrifugation (1000 RPM for 10 min).

2. The cells from each sample were processed separately in a dGH harvest procedure by exposing the cells to a 75 mM KCl hypotonic solution, followed by fixing them with a 3:1 methanol:acetic acid solution, to obtain processed cells.

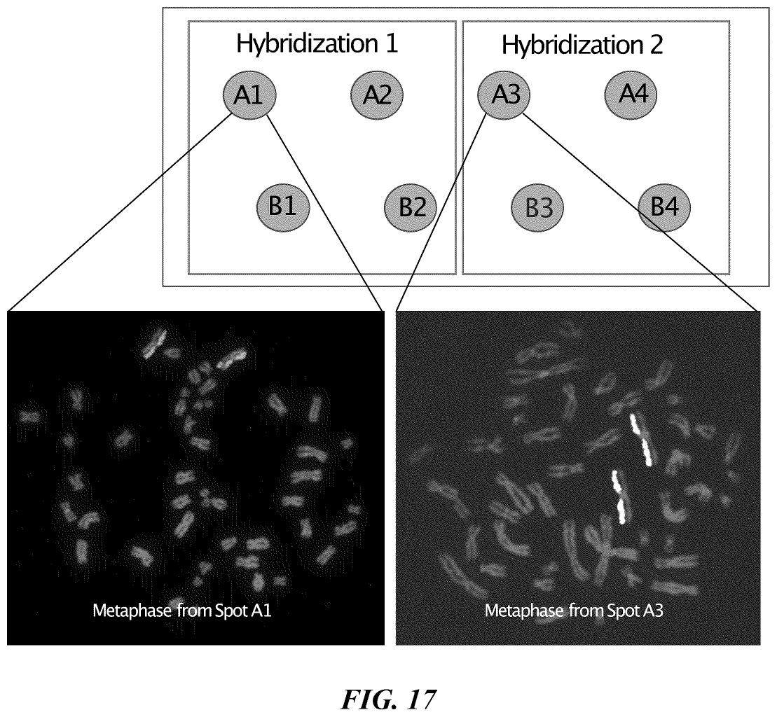

3. The processed cells from all the four samples were “spotted” onto a slide to prepare metaphase spreads in a two-dimensional, regularly spaced arrangement of the cells in a staggered configuration in two rows, with two evenly spaced 2.5 μl drops in Carnoy's fixative in each row within each of 2 partitions. Array spots A1, A2, B1, B2 (i.e., quad replicate 1) (see spots in “Hybridization 1” in the top-left panel of FIG. 17) correspond to samples 1-4 and were contained within a first physical barrier partition, and array spots A3, A4, B3, and B4 (i.e., quad replicate 2) (see spot in “Hybridization 2” in the top-right panel of FIG. 17) correspond to a second replicate of A1, A2, B1, and B2, respectively, and were contained within a second physical barrier partition.

4. Sample drops were dried on a flat slide at room temperature, and observed on a microscope for clean drop boundaries.

5. Slides were aged overnight, and taken through standard sample pretreatment steps for dGH hybridization. As per one non-limiting example, standard sample pretreatment steps for dGH hybridization includes selective photolysis by UV treatment of metaphase spread preparations singly substituted with bromodeoxynucleotides to form nicked DNA, followed by exonucleolytic degradation of the nicked DNA to remove the newly replicated strand in each metaphase chromosome to form single-stranded sister chromatids, as described in Ray et al. (F. A. Ray et al., “Directional genomic hybridization for chromosomal inversion discovery and detection,” Chromosome Res, vol. 21, no. 2, pp. 165-74, April 2013).

### Hybridization and Imaging:

A. Two separate hybridization reactions (FIG. 17, see “Hybridization 1” and “Hybridization 2”), one with a Chromosome 1 dGH paint with 1 dGH probe labeled in ATTO 550, and the other with a Chromosome 3 dGH paint with 1 dGH probe labeled with Texas Red, were applied to each quad replicate.

B. Array was hybridized overnight, counterstained with DAPI, and metaphases from each spot (A1-B4) were imaged using filters and excitations for ATTO 550, Texas Red, and DAPI.

### Results

A low magnification (10×) overview of the array exposed for DAPI excitation was generated and examined for clean array spot boundaries and interfaces between spots. No errant cell migration between spots was observed. After dGH probe hybridization with the metaphase spreads on the array, metaphases from each spot were imaged and examined for the presence of the correct single dGH paints as denoted in the assay configuration. The dGH analysis was successful on each of the array spots. Exemplary fluorescence images from spots A1 (see image labeled “Metaphase from spot A1” in the bottom-left panel of FIG. 17) and A3 (see image labeled “Metaphase from spot A3” in the bottom-right panel of FIG. 17) are shown. Thus, this Example demonstrates that a target chromosome can be detected using dGH analysis of dGH processed cells spotted in a two-dimensional, regularly spaced array configuration.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the inventions claimed. Thus, it should be understood that although the present aspects and embodiments have been specifically disclosed, exemplary aspects and optional features, modification and variation of the concepts herein disclosed may be resorted to by those skilled in the art, and that such modifications and variations are considered to be within the scope of the present disclosure. The specific aspects and embodiments provided herein are examples of useful aspects and embodiments and it will be apparent to one skilled in the art that the aspects and embodiments herein may be carried out using a large number of variations of the devices, device components, methods steps set forth in the present description. As will be obvious to one of skill in the art, methods and devices useful for the present methods can include a large number of optional composition and processing elements and steps.

All patents and publications mentioned in the specification are indicative of the levels of skill of those skilled in the art to which the present disclosure pertains. References cited herein are incorporated by reference herein in their entirety to indicate the state of the art as of their publication or filing date and it is intended that this information can be employed herein, if needed, to exclude specific aspects that are in the prior art.

One of ordinary skill in the art will appreciate that starting materials, biological materials, reagents, synthetic methods, purification methods, analytical methods, assay methods, and biological methods other than those specifically exemplified can be employed in the practice of aspects and embodiments herein, without resort to undue experimentation. All art-known functional equivalents of any such materials and methods are intended to be included in this disclosure. The terms and expressions which have been employed are used as terms of description and not of limitation, and there is no intention in the use of such terms and expressions of excluding any equivalents of the features shown and described or portions thereof, but it is recognized that various modifications are possible within the scope of the inventions claimed. Thus, it should be understood that although the present aspects and embodiments have been specifically disclosed, modification and variation of the concepts herein disclosed may be resorted to by those skilled in the art, and that such modifications and variations are considered to be within the scope of this disclosure as defined by the appended claims.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the aspects and embodiments herein as set forth in the claims below. Accordingly, the specification and figures are to be regarded in an illustrative rather than a restrictive sense, and all such modifications are intended to be included within the scope of aspects and embodiments disclosed herein.

What is claimed is:

1. A method for analyzing cells in a cell population, comprising
a) sorting cells in the cell population using a fluorescence-based cell sorting method, to increase the proportion of cells in metaphase, thereby providing a metaphase-enriched cell population;
b) contacting one or both of a pair of single-stranded sister chromatids from individual cells of the metaphase-enriched cell population, with a first dGH probe, the first dGH probe comprising a first colored fluorescent label of a set of fluorescent labels, wherein the first dGH probe comprises a pool of single-stranded oligonucleotides that comprise a same fluorescent label of the set of fluorescent labels, wherein each single stranded oligonucleotide of a pool binds a different complementary DNA sequence within a same target DNA sequence found on one of the single-stranded sister chromatids, and wherein the single-stranded sister chromatids are prepared by degrading a strand of a sister chromatid; and
c) detecting the first colored fluorescent label, to detect at least one structural feature if present on a chromosome of the individual cells, thereby analyzing cells in the cell population.

2. The method of claim 1, wherein the contacting further comprises contacting the one or both of the pair of single-stranded sister chromatids with a second dGH probe labeled with a second colored fluorescent label, wherein each single-stranded oligonucleotide of the second dGH probe is complementary to a portion of a second target DNA sequence on one of the single-stranded sister chromatids and comprises a second colored fluorescent label, and the detecting is detecting a spectral profile generated based on a hybridization pattern of the of the first dGH and the second dGH probe to one or both single stranded sister chromatids.

3. The method of claim 2, wherein the detecting the spectral profile comprises:
d) (i) comparing the spectral profile of the one or both single-stranded sister chromatids to a reference spectral profile representing a control sequence; and
d) (ii) detecting at least one difference between the reference spectral profile and the spectral profile of the one or both single-stranded sister chromatids of the pair.

4. The method of claim 3, wherein the method further comprises before the contacting, placing the cells in a two-dimensional, regularly spaced arrangement on a support matrix.

5. The method of claim 3, wherein the structural feature is a structural variation.

6. A method for detecting at least one structural feature in a chromosome of individual cells of a cell population, the method comprising the steps of:
a) applying a fluorescence-based cell sorting method to the cell population to generate a sorted subpopulation of cells, wherein the cell sorting is based on a cell cycle stage, the presence of one or more target cell surface markers, the presence of one of one or more specific chromosomes, the presence of a target DNA sequence or a set thereof, or the presence of a structural feature on the chromosome
b) contacting a pair of single-stranded sister chromatids in a metaphase spread prepared from individual cells of the metaphase-enriched cell population, with a first dGH probe, wherein each single-stranded sister chromatid is prepared by degrading a chromosome strand, wherein the first dGH probe comprising a first colored fluorescent label of a set of fluorescent labels, wherein the first dGH probe comprises a pool of single-stranded oligonucleotides that comprise a same fluorescent label of the set of fluorescent labels, and wherein each single stranded oligonucleotide of a pool binds a different complementary DNA sequence within a same target DNA sequence found on one of the single-stranded sister chromatids;
c) generating a spectral profile from one or both single-stranded sister chromatids using fluorescence detection, wherein the spectral profile is based on a hybridization pattern of the first dGH probe to one or both single-stranded sister chromatids of the pair; and
d) detecting based on the spectral profile, the presence of the at least one structural feature if present on the chromosome from individual cells.

7. The method of claim 6, wherein the contacting further comprises contacting the one or both of the pair of single-stranded sister chromatids with a second dGH probe labeled with a second colored fluorescent label, wherein each single-stranded oligonucleotide of the second dGH probe is complementary to a portion of a second target DNA sequence on one of the single-stranded sister chromatids and comprises a second colored fluorescent label, and the detecting is detecting the spectral profile generated based on the hybridization pattern of the of the first dGH and the second dGH probe to one or both single stranded sister chromatids.

8. The method of claim 7, wherein the detecting the spectral profile comprises:
d) (i) comparing the spectral profile of the one or both single-stranded sister chromatids to a reference spectral profile representing a control sequence; and
d) (ii) detecting at least one difference between the reference spectral profile and the spectral profile of the one or both single-stranded sister chromatids of the pair.

9. The method of claim 8, wherein the method further comprises before the contacting, placing the cells in a two-dimensional, regularly spaced arrangement on a support matrix.

10. The method of claim 8, wherein the structural feature is a structural variation.

11. A method for detecting a structural feature in a chromosome of cells in a cell population in a two-dimensional spatial arrangement, comprising the steps of:
a) placing individual cells from the cell population in a two-dimensional, regularly spaced arrangement on a support matrix, wherein the cell population is a metaphase-enriched cell population
b) generating a pair of single-stranded sister chromatids from a chromosome for each of the cells by degrading a strand from sister chromatids, wherein at least one of the sister chromatids comprises a target DNA sequence;
c) contacting one or both of a pair of single-stranded sister chromatids in individual cells of the cell population, with a first directional genomic hybridization (dGH) probe, wherein the first dGH probe comprises a pool of single-stranded oligonucleotides complementary to a portion of a first target DNA sequence on one of the single-stranded sister chromatids and comprising a first colored fluorescent label; and
d) performing fluorescence analysis of one or both single-stranded sister chromatids by detecting a spectral profile generated based on a hybridization pattern of the first dGH probe to one of the single-stranded sister chromatids of the pair, thereby detecting the structural feature if present on the chromosome.

12. The method of claim 11, wherein the method further comprises sorting cells using a fluorescence-based sorting method to generate the metaphase-enriched cell population.

13. The method of claim 11, wherein the contacting further comprises contacting the one or both of the pair of single-stranded sister chromatids with a second dGH probe labeled with a second colored fluorescent label, wherein each single-stranded oligonucleotide of the second dGH probe is complementary to a portion of a second target DNA sequence on one of the single-stranded sister chromatids and comprises a second colored fluorescent label, and the detecting is detecting the spectral profile generated based on the hybridization pattern of the of the first dGH and the second dGH probe to one or both single stranded sister chromatids.

14. The method of claim 13, wherein the detecting the spectral profile comprises:
d) (i) comparing the spectral profile of the one or both single-stranded sister chromatids to a reference spectral profile representing a control sequence; and
d) (ii) detecting at least one difference between the reference spectral profile and the spectral profile of the one or both single-stranded sister chromatids of the pair.

15. The method of claim 14, wherein the structural feature is a structural variation.

16. A method for a two-dimensional spatial arrangement of chromosomes from a cell population and detection of at least one structural feature in the chromosomes, comprising the steps of:
a) placing the chromosomes into a two-dimensional, regularly spaced arrangement on a solid support;
b) contacting on the solid support, one or both of a pair of single-stranded sister chromatids generated from each of the chromosomes, with a first directional genomic hybridization (dGH) probe, wherein each single-stranded sister chromatid is prepared by degrading a chromosome strand, and wherein each dGH probe comprises a pool of single-stranded oligonucleotides complementary to a portion of a first target DNA sequence on one of the single-stranded sister chromatids and comprising a first colored fluorescent label;
c) performing fluorescence analysis of one or both single-stranded sister chromatids by detecting a spectral profile generated based on a hybridization pattern of the first dGH probe to one of the single-stranded sister chromatids of the pair; and
d) detecting the spectral profile of one or both single-stranded sister chromatid on the solid support, thereby detecting if present, the at least one structural feature.

17. The method of claim 16, wherein the contacting further comprises contacting the one or both of the pair of single-stranded sister chromatids with a second dGH probe labeled with a second colored fluorescent label, wherein each single-stranded oligonucleotide of the second dGH probe is complementary to a portion of a second target DNA sequence on one of the single-stranded sister chromatids and comprises a second colored fluorescent label, and the detecting is detecting the spectral profile generated based on the hybridization pattern of the of the first dGH and the second dGH probe to one or both single stranded sister chromatids.

18. The method of claim 17, wherein the detecting the spectral profile comprises:
d) (i) comparing the spectral profile of the one or both single-stranded sister chromatids to a reference spectral profile representing a control sequence; and
d) (ii) detecting at least one difference between the reference spectral profile and the spectral profile of the one or both single-stranded sister chromatids of the pair.

19. The method of claim 18, wherein the structural feature is a structural variation.

20. The method of claim 18, wherein the method further comprises sorting cells using a fluorescence-based sorting method on the cell population to generate a metaphase-enriched cell population comprising the chromosomes, and isolating the chromosomes from the metaphase-enriched cell population using a dGH harvest procedure, before placing the chromosomes on the solid support.

21. The method of any one of claims 1, 6, 11, or 16, wherein the detecting the spectral profile comprises:
d) (i) comparing the spectral profile of the one or both single-stranded sister chromatids to a reference spectral profile representing a control sequence; and
d) (ii) detecting at least one difference between the reference spectral profile and the spectral profile of the one or both single-stranded sister chromatids of the pair.

22. The method of any one of claims 1, 6, 11, or 16, wherein the method further comprises before the contacting, placing the cells in a two-dimensional, regularly spaced arrangement on a support matrix.

23. The method of any one of claims 1, 6, 11, or 16, wherein the structural feature is a structural variation.

24. The method of any one of claims 1 to 20, wherein the method further comprises labeling chromatids that comprise the single-stranded sister chromatids with a DNA stain.

25. The method of any one of claims 1 to 10, 12, and 20, wherein the fluorescence-based cell sorting method uses a DNA stain to generate the sorted population of cells.

26. The method of claim 25, wherein the DNA stain is a fluorochrome.

27. The method of claim 25, wherein the DNA stain is a fluorochrome that binds chromosomes through DNA intercalation or that binds a secondary structure of DNA.

28. The method of claim 25, wherein the DNA stain is selected from the group consisting of propidium iodine, 7-AAD, a Hoechst stain, chromomycin A3, quinacrine, and daunomycin.

29. The method of claim 25, wherein the DNA stain is a Hoechst stain.

30. The method of any one of claims 1 to 20, further wherein the method further comprises, collecting other single-stranded sister chromatids from the cell population or chromosomes, and sequencing at least one nucleic acid generated from the collected other single-stranded sister chromatids.

31. The method of any one of claims 1 to 20, further wherein the method further comprises, collecting other single-stranded sister chromatids from the cell population and sequencing at least one nucleic acid generated from the collected other single-stranded sister chromatids.

32. The method of claim 31, wherein the nucleic acid sequencing is single-cell template strand sequencing.

33. The method of claim 32, wherein the degrading is performed by incorporating a DNA analog into genomic DNA of the individual cells of the population of cells for one cell cycle, and degrading the newly synthesized chromosome strand that incorporates the DNA analog.

34. The method of claim 33, wherein the DNA analog is a uridine analog.

35. The method of claim 34, wherein method further comprises staining the single-stranded sister chromatids with a DNA stain.

36. The method of claim 35, wherein the DNA stain is an intercalating dye that preferentially binds to double-stranded DNA at A-T sites.

37. The method of claim 36, wherein the DNA stain is a Hoechst stain.

38. The method of any one of claims 1 to 20, wherein the degrading is performed by incorporating a DNA analog into genomic DNA of the individual cells of the population of cells for one cell cycle, and degrading the newly synthesized chromosome strand that incorporates the DNA analog.

39. The method of claim 38, wherein the DNA analog is a uridine analog.

40. The method of claim 39, wherein method further comprises staining the single-stranded sister chromatids with a DNA stain.

41. The method of claim 40, wherein the DNA stain is an intercalating dye that preferentially binds to double-stranded DNA at A-T sites.

42. The method of claim 41, wherein the DNA stain is a Hoechst stain.

43. The method according to any one of claims 1 to 20, wherein one or more cell of the population of cells expresses a reporter protein.

44. The method of any one of claims 1 to 20, wherein the cell sorting method further comprises sorting cells based on one or more cell surface expressed markers.

45. The method of any one of claims 1 to 10, 12, and 20, wherein the fluorescence cell sorting method is an automated fluorescence cell sorting method.

46. The method according to any one of claims 4, 9, or 11 to 20, wherein the solid support comprises two or more physical barrier partitions each comprising the two-dimensional, regularly spaced arrangement of the cells or chromosomes.

47. The method according to any claim 46, wherein the cell population is derived from more than one cell sample, and wherein the cells or the chromosomes from different samples of the more than one cell sample are placed on the solid support within different partitions.

48. The method of claim 45, wherein one physical barrier partition contains the first dGH probe and another physical barrier partition contains the second dGH probe during performance of the method.

49. The method of claim 46, wherein the two-dimensional, regularly spaced arrangement is an array with at least 96 selected, separated locations.

50. The method of claim 49, wherein cell population comprises cells from at least 96 samples, wherein cells from each sample are placed in a separate partition on the solid support.

51. The method of claim 49, wherein the array comprises single-stranded chromatids from over 40,000 metaphase-enriched cells.

52. The method of any one of claims 4, 9, or 11 to 20, wherein the two-dimensional, regularly spaced arrangement is an array with at least 96 selected, separated locations.

53. The method of claim 52, wherein the solid support comprises at least 6 rows of partitions, each comprising the two-dimensional, regularly spaced arrangement.

54. The method of any one of claims 4, 9, or 11 to 20, wherein the solid support comprises at least 6 rows of partitions, each comprising the two-dimensional, regularly spaced arrangement.

55. The method of any one of claims 1 to 20, wherein the first target DNA sequence is unique in the genome of the cells.

56. The method of any one of claims 1 to 20, wherein the complementary sequence for every probe used in the contacting is unique in the genome of the cells.

57. The method according to any one of claims 2, 7, 13, or 17, wherein the spectral profile provides a banding pattern on the at least one single-stranded sister chromatid comprising bands of different colors.

58. The method of claim 57, wherein between 2 and 10 dGH probes are used in the method and the banding pattern on the at least one single-stranded sister chromatid comprises bands of between 2 and 10 different colors.
